SEQUENCE LISTING (131 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 3 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 4 - protein, 99 aa, Homo sapiens [sequence omitted]

SEQ ID NO: 5 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 6 - protein, 341 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 7 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 8 - protein, 338 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 9 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 10 - protein, 342 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 11 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 12 - protein, 337 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 13 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 14 - protein, 325 aa, Homo sapiens
GSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 15 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 16 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 17 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 18 - protein, 278 aa, Homo sapiens
QENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 19 - protein, 348 aa, Homo sapiens
MGPRARPALLLLMLLQTAVLQGRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIWEPSPSGTLVIGVISGIAVFVVILFIGILFIILRKRQGSRGAMGHYVLAERE

SEQ ID NO: 20 - protein, 326 aa, Homo sapiens
RLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIWEPSPSGTLVIGVISGIAVFVVILFIGILFIILRKRQGSRGAMGHYVLAERE

SEQ ID NO: 21 - protein, 341 aa, Homo sapiens
MGELMAFLLPLIIVLMVKHSDSRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDGQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHCGVHMVLQVPQESETIPLVMKAVSGSIVLVIVLAGVGVLVWRRRPREQNGAIYLPTPDR

SEQ ID NO: 22 - protein, 319 aa, Homo sapiens
RTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDGQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHCGVHMVLQVPQESETIPLVMKAVSGSIVLVIVLAGVGVLVWRRRPREQNGAIYLPTPDR

SEQ ID NO: 23 - protein, 327 aa, Homo sapiens
MLFLLLPLLAVLPGDGNADGLKEPLSFHVTWIASFYNHSWKQNLVSGWLSDLQTHTWDSNSSTIVFLCPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 24 - protein, 309 aa, Homo sapiens
DGLKEPLSFHVTWIASFYNHSWKQNLVSGWLSDLQTHTWDSNSSTIVFLCPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 25 - protein, 333 aa, Homo sapiens
MLLLPFQLLAVLFPGGNSEHAFQGPTSFHVIQTSSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRNPTSIGSIVLAIIVPSLLLLLCLALWYMRRRSYQNIP

SEQ ID NO: 26 - protein, 316 aa, Homo sapiens
SEHAFQGPTSFHVIQTSSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRNPTSIGSIVLAIIVPSLLLLLCLALWYMRRRSYQNIP

SEQ ID NO: 27 - protein, 333 aa, Homo sapiens
MLFLQFLLLALLLPGGDNADASQEHVSFHVIQIFSFVNQSWARGQGSGWLDELQTHGWDSESGTIIFLHNWSKGNFSNEELSDLELLFRFYLFGLTREIQDHASQDYSKYPFEVQVKAGCELHSGKSPEGFFQVAFNGLDLLSFQNTTWVPSPGCGSLAQSVCHLLNHQYEGVTETVYNLIRSTCPRFLLGLLDAGKMYVHRQVRPEAWLSSRPSLGSGQLLLVCHASGFYPKPVWVTWMRNEQEQLGTKHGDILPNADGTWYLQVILEVASEEPAGLSCRVRHSSLGGQDIILYWGHHFSMNWIALVVIVPLVILIVLVLWFKKHCSYQDIL

SEQ ID NO: 28 - protein, 316 aa, Homo sapiens
NADASQEHVSFHVIQIFSFVNQSWARGQGSGWLDELQTHGWDSESGTIIFLHNWSKGNFSNEELSDLELLFRFYLFGLTREIQDHASQDYSKYPFEVQVKAGCELHSGKSPEGFFQVAFNGLDLLSFQNTTWVPSPGCGSLAQSVCHLLNHQYEGVTETVYNLIRSTCPRFLLGLLDAGKMYVHRQVRPEAWLSSRPSLGSGQLLLVCHASGFYPKPVWVTWMRNEQEQLGTKHGDILPNADGTWYLQVILEVASEEPAGLSCRVRHSSLGGQDIILYWGHHFSMNWIALVVIVPLVILIVLVLWFKKHCSYQDIL

SEQ ID NO: 29 - protein, 335 aa, Homo sapiens
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

SEQ ID NO: 30 - protein, 316 aa, Homo sapiens
EVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

SEQ ID NO: 31 - protein, 388 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 32 - protein, 369 aa, Homo sapiens
APQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 33 - protein, 11 aa, Homo sapiens
PKEFNAETFTF

SEQ ID NO: 34 - protein, 11 aa, Homo sapiens
PPAFSDDKFIF

SEQ ID NO: 35 - protein, 11 aa, Homo sapiens
KLVAASQAALG

SEQ ID NO: 36 - protein, 11 aa, Homo sapiens
KLISKTRAALG

SEQ ID NO: 37 - protein, 8 aa, Homo sapiens
LEKYGHSD

SEQ ID NO: 38 - protein, 8 aa, Homo sapiens
PTPASIPL

SEQ ID NO: 39 - protein, 14 aa, Homo sapiens
PQLTSSELMAITRK

SEQ ID NO: 40 - protein, 11 aa, Homo sapiens
HLCIRHEMTPV

SEQ ID NO: 41 - protein, 42 aa, Homo sapiens
ETYVPPAFSDDKFIFHKDLCQAQGVALQTMKQEFLINLVKQK

SEQ ID NO: 42 - protein, 9 aa, Homo sapiens
IQRTPKIQV

SEQ ID NO: 43 - protein, 13 aa, Homo sapiens
NCYVSGFHPSDIE

SEQ ID NO: 44 - protein, 23 aa, Homo sapiens
NGERIEKVEHSDLSFSKDWSFYL

SEQ ID NO: 45 - protein, 16 aa, Homo sapiens
RVNHVTLSQPKIVKWD

SEQ ID NO: 46 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 47 - protein, 335 aa, Homo sapiens; identical to SEQ ID NO: 29

SEQ ID NO: 48 - protein, 388 aa, Homo sapiens; identical to SEQ ID NO: 31

SEQ ID NO: 49 - protein, 333 aa, Homo sapiens; identical to SEQ ID NO: 27

SEQ ID NO: 50 - protein, 327 aa, Homo sapiens; identical to SEQ ID NO: 23

SEQ ID NO: 51 - protein, 333 aa, Homo sapiens; identical to SEQ ID NO: 25

SEQ ID NO: 52 - protein, 298 aa, Homo sapiens; identical to SEQ ID NO: 17

SEQ ID NO: 53 - protein, 309 aa, Homo sapiens
SHSLHYLFMGASEQDLGLSLFEALGYVDDQLFVFYDDESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTYQVEHPGLDQPLIVIWEPSPSGTLVIGVISGIAVFVVILFIGILFIILRKRQGS

SEQ ID NO: 54 - protein, 334 aa, Homo sapiens
LLPLIIVLMVKHSDSRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDGQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHCGVHMVLQVPQESETIPLVMKAVSGSIVLVIVLAGVGVLVWRRRPREQNGAIYLPTPDR

SEQ ID NO: 55 - protein, 358 aa, Homo sapiens [sequence omitted]

SEQ ID NO: 56 - protein, 362 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPIVGIVAGLVVLGAVVTGAVVAAVMWRKKSSDRNRGSYSQAAVTDSAQGSGVSLTANKV

SEQ ID NO: 57 - protein, 280 aa, Homo sapiens [sequence omitted]

SEQ ID NO: 58 - protein, 273 aa, Homo sapiens
SHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 59 - protein, 362 aa, Homo sapiens [sequence omitted]

SEQ ID NO: 60 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 61 - protein, 28 aa, Homo sapiens
ASQKMEPRAPWIEQEGPEYWDQETRNMK

SEQ ID NO: 62 - protein, 28 aa, Homo sapiens
ASPREEPRAPWIEQEGPEYWDRETQICK

SEQ ID NO: 63 - protein, 28 aa, Homo sapiens
ASPRGEPRAPWVEQEGPEYWDRETQKYK

SEQ ID NO: 64 - protein, 28 aa, Homo sapiens
ASPRMVPRAPWMEQEGSEYWDRETRSAR

SEQ ID NO: 65 - protein, 28 aa, Homo sapiens
ASPRMEPRAPWVEQEGPEYWEEETRNTK

SEQ ID NO: 66 - protein, 29 aa, Homo sapiens
ESRRVEPRTPWVSSRISSQMWLQLSQSLK

SEQ ID NO: 67 - protein, 28 aa, Homo sapiens
TRQKEPRAPWMAENLAPDHWERYTQLLR

SEQ ID NO: 68 - protein, 28 aa, Homo sapiens
DRKSQPMGLWRQVEGMEDWKQDSQLQKA

SEQ ID NO: 69 - protein, 31 aa, Homo sapiens
DSNSSTIVFLWPWSRGNFSNEEWKELETLFR

SEQ ID NO: 70 - protein, 31 aa, Homo sapiens
DSDSGTAIFLKPWSKGNFSDKEVAELEEIFR

SEQ ID NO: 71 - protein, 27 aa, Homo sapiens
DTVRSLKPWSQGTFSDQQWETLQHIFR

SEQ ID NO: 72 - protein, 28 aa, Homo sapiens
EQRRVYLEGRCVDGLRRYLENGKETLQR

SEQ ID NO: 73 - protein, 39 aa, Homo sapiens
TQRKWEAARVAEQLRAYLEGECVEWLRRYLENGKETLQR

SEQ ID NO: 74 - protein, 29 aa, Homo sapiens
EQLRAYLEGLCVEWLRRYLKNGKETLQRA

SEQ ID NO: 75 - protein, 28 aa, Homo sapiens
EHQRAYLEDTCVEWLHKYLEKGKETLLH

SEQ ID NO: 76 - protein, 28 aa, Homo sapiens
EQRRAYLEGTCVEWLHRYLENGKEMLQR

SEQ ID NO: 77 - protein, 31 aa, Homo sapiens
IRARQNRAYLERDCPAQLQQLLELGRGVLDQ

SEQ ID NO: 78 - protein, 33 aa, Homo sapiens
HELLYQKNWLEEECIAWLKRFLEYGKDTLQRTE

SEQ ID NO: 79 - protein, 32 aa, Homo sapiens
PVYVQRAKAYLEEECPATLRKYLKYSKNILDR

SEQ ID NO: 80 - protein, 33 aa, Homo sapiens
HENDITHNLLSDTCPRFILGLLDAGKAHLQRQV

SEQ ID NO: 81 - protein, 30 aa, Homo sapiens
ETVRILLYETCPRYLLGVLNAGKADLQRQV

SEQ ID NO: 82 - protein, 32 aa, Homo sapiens
WTRETVQWLLNGTCPQFVSGLLESGKSELKKQ

SEQ ID NO: 83 - protein, 6 aa, Homo sapiens
PTPASI

SEQ ID NO: 84 - protein, 8 aa, Homo sapiens
LEKYGHSD

SEQ ID NO: 85 - protein, 8 aa, Homo sapiens
LEKYGHSD

SEQ ID NO: 86 - protein, 7 aa, Homo sapiens
PTPASIP

SEQ ID NO: 87 - protein, 8 aa, Homo sapiens
LEKYGHSD

SEQ ID NO: 88 - protein, 5 aa, Homo sapiens
PTPAS

SEQ ID NO: 89 - protein, 8 aa, Homo sapiens
LEKYGHSD

SEQ ID NO: 90 - protein, 5 aa, Homo sapiens
PTPAS

SEQ ID NO: 91 - protein, 11 aa, Homo sapiens
LEKYGHSDCCS

SEQ ID NO: 92 - protein, 7 aa, Homo sapiens
PTPASIP

SEQ ID NO: 93 - protein, 8 aa, Homo sapiens
LEKYGHSD

SEQ ID NO: 94 - protein, 8 aa, Homo sapiens
KPTPASIP

SEQ ID NO: 95 - protein, 8 aa, Homo sapiens
LEKYGHSD

SEQ ID NO: 96 - protein, 7 aa, Homo sapiens
PTPASIP

SEQ ID NO: 97 - protein, 8 aa, Homo sapiens
LEKYGHSD

SEQ ID NO: 98 - protein, 7 aa, Homo sapiens
PTPASIP

SEQ ID NO: 99 - protein, 10 aa, Homo sapiens
PQLTSSELMA

SEQ ID NO: 100 - protein, 7 aa, Homo sapiens
PQLTSSE

SEQ ID NO: 101 - protein, 7 aa, Homo sapiens
PQLTSSE

SEQ ID NO: 102 - protein, 7 aa, Homo sapiens
PQLTSSE

SEQ ID NO: 103 - protein, 10 aa, Homo sapiens
PQLTSSELMA

SEQ ID NO: 104 - protein, 10 aa, Homo sapiens
PQLTSSELMA

SEQ ID NO: 105 - protein, 9 aa, Homo sapiens
QLTSSELMA

SEQ ID NO: 106 - protein, 7 aa, Homo sapiens
PQLTSSE

SEQ ID NO: 107 - protein, 9 aa, Homo sapiens
QLTSSELMA

SEQ ID NO: 108 - protein, 10 aa, Homo sapiens
PQLTSSELMA

SEQ ID NO: 109 - protein, 9 aa, Homo sapiens
QLTSSELMA

SEQ ID NO: 110 - protein, 10 aa, Homo sapiens
LCIRHEMTPV

SEQ ID NO: 111 - protein, 10 aa, Homo sapiens
LCIRHEMTPV

SEQ ID NO: 112 - protein, 11 aa, Homo sapiens
HLCIRHEMTPV

SEQ ID NO: 113 - protein, 7 aa, Homo sapiens
RHEMTPV

SEQ ID NO: 114 - protein, 11 aa, Homo sapiens
HLCIRHEMTPV

SEQ ID NO: 115 - protein, 9 aa, Homo sapiens
RHEMTPVNP

SEQ ID NO: 116 - protein, 12 aa, Homo sapiens
LCIRHEMTPVNP

SEQ ID NO: 117 - protein, 7 aa, Homo sapiens
RHEMTPV

SEQ ID NO: 118 - protein, 11 aa, Homo sapiens
HLCIRHEMTPV

SEQ ID NO: 119 - protein, 10 aa, Homo sapiens
LCIRHEMTPV

SEQ ID NO: 120 - protein, 11 aa, Homo sapiens
HLCIRHEMTPV

SEQ ID NO: 121 - protein, 39 aa, Homo sapiens
TYVPPAFSDDKFIFHKDLCQAQGVALQTMKQEFLINLVK

SEQ ID NO: 122 - protein, 39 aa, Homo sapiens; identical to SEQ ID NO: 121

SEQ ID NO: 123 - protein, 39 aa, Homo sapiens; identical to SEQ ID NO: 121

SEQ ID NO: 124 - protein, 39 aa, Homo sapiens; identical to SEQ ID NO: 121

SEQ ID NO: 125 - protein, 39 aa, Homo sapiens; identical to SEQ ID NO: 121

SEQ ID NO: 126 - protein, 39 aa, Homo sapiens; identical to SEQ ID NO: 121

SEQ ID NO: 127 - protein, 33 aa, Homo sapiens
TYVPPAFSDDKFIFHKDLCQAQGVALQTMKQEF

SEQ ID NO: 128 - protein, 39 aa, Homo sapiens; identical to SEQ ID NO: 121

SEQ ID NO: 129 - protein, 32 aa, Homo sapiens
YVPPAFSDDKFIFHKDLCQAQGVALQTMKQEF

SEQ ID NO: 130 - protein, 33 aa, Homo sapiens; identical to SEQ ID NO: 127

SEQ ID NO: 131 - protein, 20 aa, Homo sapiens
TYVPPAFSDDKFIFHKDLCQ

(19) United States
(12) Patent Application Publication
(10) Pub. No.: US 2019/0201496 A1
(43) Pub. Date: Jul. 4, 2019

(54) BLOCKADE OF ALPHAFETOPROTEIN (AFP) INTERACTIONS WITH BETA2-MICROGLOBULIN ASSOCIATED MOLECULES

(71) Applicants: THE BRIGHAM AND WOMEN'S HOSPITAL, INC., Boston, MA (US); UNIVERSITY OF OSLO, Oslo (NO)

(72) Inventors: Richard S. BLUMBERG, Waltham, MA (US); Michal PYZIK, Cambridge, MA (US); Amit GANDHI, Billerica, MA (US); Inger SANDLIE, Oslo (NO); Kine Marita Knudsen SAND, Oslo (NO); Jan Terje ANDERSEN, Hallagerbakken (NO)

(73) Assignees: THE BRIGHAM AND WOMEN'S HOSPITAL, INC., Boston, MA (US); UNIVERSITY OF OSLO, Oslo (NO)

(21) Appl. No.: 16/333,918

(22) PCT Filed: Sep. 14, 2017
(86) PCT No.: PCT/US2017/051462

§ 371 Date: Mar. 15, 2019

Related U.S. Application Data
(60) Provisional application No. 62/395,696, filed on Sep. 16, 2016.

(51) Int. Cl. A61K 38/38; C07K 14/47; C12P 21/02; A61P 37/06; C07K 14/765

(52) U.S. Cl. CPC A61K 38/38; C07K 14/47; C07K 14/765; A61P 37/06; C12P 21/02

(57) ABSTRACT

Provided herein, in some aspects, are compositions and methods to inhibit AFP interactions with β2M and/or Class I-related molecule interactions in diseases or disorders where elevated AFP levels are associated with immunosuppression. Also provided herein, in some aspects, are compositions and methods to enhance or potentiate AFP interactions with β2M and/or Class I-related molecule in diseases or disorders with decreased AFP levels or diseases or disorders where increasing AFP levels is desired to increase immunosuppression or enhance organ regeneration.

### CROSS-REFERENCE TO RELATED APPLICATIONS

This application claims benefit under 35 U.S.C. § 119(e) of U.S. Provisional Application Ser. No. 62/395,696 filed Sep. 16, 2016, the content of which is incorporated herein by reference in its entirety.

### GOVERNMENT SUPPORT

This invention was made with government support under DK-53056 awarded by the National Institutes of Health (NIH). The government has certain rights in the invention

### TECHNICAL FIELD

The technical field relates to compositions and methods for modulating alpha-fetoprotein levels and activities.

### BACKGROUND

Alpha-fetoprotein (AFP) is a major plasma protein in the fetus, where it is produced by the yolk sac and liver (Ingram et al., 1981). In an adult, its concentration is very low, except when a tumor, such as a hepatoma or teratoma, is present. The alpha-fetoprotein and albumin genes are syntenic, and mammalian AFP and serum albumin genes are believed to have arisen through duplication of an ancestral gene 300 to 500 million years ago.

### SUMMARY

The compositions and methods described herein are based, in part, on the discovery that alpha-fetoprotein (AFP) binds to β2-microglobulin (also referred to herein as “β2M” and “BMG) and to Major Histocompatibility Class (MHC) class I-related molecules, and that the binding of AFP to MHC class I-related molecules is stabilized by the binding of AFP to β2M. AFP is normally produced by the embryonic yolk sac and the fetal liver, but is expressed at elevated levels post-natally in pathological conditions, including cancer (hepatocellular carcinoma, cholangiocarcinoma, teratocarcinoma, among other) and certain inflammatory conditions. AFP and peptides derived from it have immunosuppressive activity and have been posited as potentially useful, e.g., for the treatment of autoimmune diseases and transplant rejection (see, e.g., U.S. Pat. Nos. 7,423,024, 6,774,108, 6,288,034 and 5,965,528), among other pathologies.

AFP expressed in cancer is believed to participate in or facilitate tumor immune evasion, making AFP a target for cancer immunotherapy in those cancers that express or are otherwise associated with AFP expression and/or elevated serum AFP levels. Inhibition of AFP-mediated immunosuppression in the tumor environment can permit cell-mediated immune attack on the tumor. It was previously discovered that AFP binds the neonatal Fc receptor (FcRn) and β2M, and makes a ternary complex. As described herein, the inventors have now discovered that AFP also binds to β2M alone and this binding allows AFP to associate with other members of the MHC class I-related family of molecules, such as, HLA-A, that have structural similarity to FcRn. Analyses of the structure and binding properties of AFP:HLA-A indicate that the HLA-A structure that binds AFP is shared in other MHC class I-related molecules that participate in antigen presentation to cytotoxic T cells. As such, the immunosuppressive activity of AFP can be mediated not only by the AFP-FcRn interaction or the AFP-HLA-A interaction, but can also be mediated by the interaction of AFP with other MHC class I-related molecules, such that the immunosuppressive activity of AFP can involve its interaction with, e.g., HFE, and other classical and non-classical MHC class I-related molecules. As described further herein below, an amino acid alignment established a “phylogenetic tree” or cladogram of sorts, scoring the similarity to FcRn and MR1 to provide a hierarchy of the likely strength of interactions of MHC Class I-related proteins with AFP, such that FcRn≥HFE≥HLA-A≥HLA-G≥HLA-E≥HLA-B≥MR1≥CD1D≥HLA-C≥ZA2G≥CD1A≥CD1B. Where AFP can interact with any or all of these proteins, any or all of them may be involved in the immunosuppressive effects of AFP, and targeting such interactions can inhibit such immunosuppressive effects.

Accordingly, provided herein, in some aspects, are compositions and methods to inhibit AFP and β2M and/or AFP and MHC Class I-related molecule interactions in diseases or disorders where elevated AFP levels are associated with immunosuppression. Also provided herein, in some aspects, are compositions and methods to enhance or potentiate AFP and β2M and/or AFP and MHC Class I-related molecule interactions in diseases or disorders with decreased AFP levels or diseases or disorders where AFP levels increase with immunosuppression.

In some aspects, provided herein are pharmaceutical compositions comprising an inhibitor of alpha-fetoprotein (AFP)-β2-microglobulin (β2M) interactions and a pharmaceutically acceptable carrier, wherein said inhibitor of AFP-β2M interactions inhibits binding between AFP and β2M.

In some embodiments of these aspects and all such aspects described herein, the inhibitor of AFP-β2M interactions inhibits interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO: 4, an interface of β2M comprising amino acids 81-96 of SEQ ID NO: 4, or any combination thereof.

In some embodiments of these aspects and all such aspects described herein, the inhibitor of AFP-β2M interactions inhibits interaction of β2M with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

In some embodiments of these aspects and all such aspects described herein, the inhibition of binding between AFP and β2M further inhibits or prevents interaction or complex formation between β2M and an MHC Class I-related molecule.

In some embodiments of these aspects and all such aspects described herein, the MHC Class I-related molecule is selected from HFE, HLA-A, HLA-G, HLA-E, HLA-B, MR1, CD1D, HLA-C, ZA2G, CD1A, and CD1B.

In some embodiments of these aspects and all such aspects described herein, the inhibitor of AFP-β2M interactions is an antibody or antigen-binding fragment thereof, a small molecule compound, or an RNA or DNA aptamer.

In some embodiments of these aspects and all such aspects described herein, the antibody or antigen-binding fragment thereof is a chimeric, humanized, or completely human antibody or antigen-binding fragment thereof.

In some aspects, provided herein are pharmaceutical compositions comprising an inhibitor of alpha-fetoprotein (AFP)-MHC Class I-related interactions and a pharmaceutically acceptable carrier, wherein said inhibitor of AFP-MHC Class I-related interactions inhibits binding between AFP and an MHC Class I-related molecule.

In some embodiments of these aspects and all such aspects described herein, the MHC Class I-related molecule is selected from HFE, HLA-A, HLA-G, HLA-E, HLA-B, MR1, CD1D, HLA-C, ZA2G, CD1A, and CD1B.

In some embodiments of these aspects and all such aspects described herein, the inhibitor of AFP-MHC Class I-related interactions inhibits interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

In some embodiments of these aspects and all such aspects described herein, the inhibitor of AFP-MHC Class I-related interactions inhibits interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

In some embodiments of these aspects and all such aspects described herein, the inhibitor of AFP-MHC Class I-related interactions inhibits interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

In some embodiments of these aspects and all such aspects described herein, the inhibitor of AFP-MHC Class I-related interactions inhibits interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

In some embodiments of these aspects and all such aspects described herein, the inhibitor of AFP-MHC Class I-related interactions inhibits interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

In some embodiments of these aspects and all such aspects described herein, the inhibitor of AFP-MHC Class I-related interactions inhibits interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

In some embodiments of these aspects and all such aspects described herein, the inhibitor of AFP-MHC Class I-related interactions inhibits interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

In some embodiments of these aspects and all such aspects described herein, the inhibitor of AFP-MHC Class I-related interactions inhibits interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

In some embodiments of these aspects and all such aspects described herein, the inhibitor of AFP and MHC Class I-related molecule interactions inhibits interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

In some embodiments of these aspects and all such aspects described herein, the inhibitor of AFP and MHC Class I-related molecule interactions inhibits interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

In some embodiments of these aspects and all such aspects described herein, the inhibitor of AFP and MHC Class I-related molecule interactions inhibits interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

In some embodiments of these aspects and all such aspects described herein, the inhibitor of AFP-MHC Class I-related interactions inhibits interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

In some embodiments of these aspects and all such aspects described herein, the inhibitor of AFP-MHC Class I-related interactions inhibits interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the inhibitor of AFP-MHC Class I-related interactions inhibits interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

In some embodiments of these aspects and all such aspects described herein, the inhibitor of AFP-MHC Class I-related interactions inhibits interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

In some embodiments of these aspects and all such aspects described herein, the inhibitor of AFP-MHC Class I-related interactions inhibits interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

In some embodiments of these aspects and all such aspects described herein, the inhibitor of AFP-MHC Class I-related interactions also inhibits binding between S527 or D528 of SEQ ID NO: 2 and E50 and 67Y of β2M, respectively, complexed with an MHC Class I-related molecule.

In some embodiments of these aspects and all such aspects described herein, the inhibitor of AFP-MHC Class I-related interactions also inhibits binding between R604 of SEQ ID NO: 2 and the carbonyl oxygen at E50 of β2M, wherein the β2M is complexed with an MHC Class I-related molecule.

In some embodiments of these aspects and all such aspects described herein, the inhibitor of AFP-MHC Class I-related interactions is an antibody or antigen-binding fragment thereof, a small molecule compound, a peptide inhibitor, or an RNA or DNA aptamer.

In some embodiments of these aspects and all such aspects described herein, the antibody or antigen-binding fragment thereof is a chimeric, humanized, or completely human antibody or antigen-binding fragment thereof.

Provided herein, in some aspects, are pharmaceutical compositions comprising a potentiator of alpha-fetoprotein (AFP)-β2M (β-2-microglobulin interactions and a pharmaceutically acceptable carrier, wherein said potentiator of AFP-β2M interactions increases binding between AFP and β2M.

In some embodiments of these aspects and all such aspects described herein, the potentiator of AFP-β2M interactions increases interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO:4, an interface of β2M comprising amino acids 81-96 of SEQ ID NO: 4, or any combination thereof.

In some embodiments of these aspects and all such aspects described herein, the potentiator of AFP-β2M interactions increases interaction of AFP with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

In some embodiments of these aspects and all such aspects described herein, the increased binding between AFP and β2M further increases or enhances interaction or complex formation between β2M and an MHC Class I-related molecule.

In some embodiments of these aspects and all such aspects described herein, the MHC Class I-related molecule is selected from HFE, HLA-A, HLA-G, HLA-E, HLA-B, MR1, CD1D, HLA-C, ZA2G, CD1A, and CD1B.

In some embodiments of these aspects and all such aspects described herein, the potentiator of AFP-β2M interactions is an antibody or antigen-binding fragment thereof, a small molecule compound, or an RNA or DNA aptamer.

In some embodiments of these aspects and all such aspects described herein, the antibody or antigen-binding fragment thereof is a chimeric, humanized, or completely human antibody or antigen-binding fragment thereof.

In some aspects, provided herein are pharmaceutical compositions comprising a potentiator of AFP-MHC Class I-related molecule interactions and a pharmaceutically acceptable carrier, wherein said potentiator of AFP-MHC Class I-related molecule interactions increases binding between alpha-fetoprotein (AFP) and an MHC Class I-related molecule.

In some embodiments of these aspects and all such aspects described herein, the MHC Class I-related molecule is selected from HFE, HLA-A, HLA-G, HLA-E, HLA-B, MR1, CD1D, HLA-C, ZA2G, CD1A, and CD1B.

In some embodiments of these aspects and all such aspects described herein, the potentiator of AFP-MHC Class I-related molecule interactions increases interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

In some embodiments of these aspects and all such aspects described herein, the potentiator of AFP-MHC Class I-related molecule interactions interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

In some embodiments of these aspects and all such aspects described herein, the potentiator of AFP-MHC Class I-related interactions increases interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

In some embodiments of these aspects and all such aspects described herein, the potentiator of AFP-MHC Class I-related molecule interactions increases interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

In some embodiments of these aspects and all such aspects described herein, the potentiator of AFP-MHC Class I-related molecule interactions increases interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

In some embodiments of these aspects and all such aspects described herein, the potentiator of AFP-MHC Class I-related molecule interactions interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

In some embodiments of these aspects and all such aspects described herein, the potentiator of AFP-MHC Class I-related molecule interactions increases interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

In some embodiments of these aspects and all such aspects described herein, the potentiator of AFP-MHC Class I-related molecule interactions increases interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

In some embodiments of these aspects and all such aspects described herein, the potentiator of AFP-MHC Class I-related molecule interactions increases interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

In some embodiments of these aspects and all such aspects described herein, the potentiator of AFP-MHC Class I-related molecule interactions increases interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

In some embodiments of these aspects and all such aspects described herein, the potentiator of AFP-MHC Class I-related molecule interactions increases interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

In some embodiments of these aspects and all such aspects described herein, the potentiator of AFP-MHC Class I-related molecule interactions increases interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

In some embodiments of these aspects and all such aspects described herein, the potentiator of AFP-MHC Class I-related molecule interactions increases interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the potentiator of AFP-MHC Class I-related molecule interactions increases interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

In some embodiments of these aspects and all such aspects described herein, the potentiator of AFP-MHC Class I-related molecule interactions increases interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

In some embodiments of these aspects and all such aspects described herein, the potentiator of AFP-MHC Class I-related molecule interactions increases interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

In some embodiments of these aspects and all such aspects described herein, the potentiator of AFP-MHC Class I-related interactions also increases interaction between S527 or D528 of SEQ ID NO: 2 and E50 and 67Y of SEQ ID NO: 4, respectively, complexed with an MHC Class I-related molecule.

In some embodiments of these aspects and all such aspects described herein, the potentiator of AFP-MHC Class I-related interactions also increases interaction between R604 of SEQ ID NO: 2 and the carbonyl oxygen at E50 of SEQ ID NO: 4, wherein the β2M is complexed with an MHC Class I-related molecule.

In some embodiments of these aspects and all such aspects described herein, the potentiator of AFP-MHC Class I-related interactions is an antibody or antigen-binding fragment thereof, a small molecule compound, or an RNA or DNA aptamer.

In some embodiments of these aspects and all such aspects described herein, the antibody or antigen-binding fragment thereof is a chimeric, humanized, or completely human antibody or antigen-binding fragment thereof.

Provided herein, in some aspects, are methods to inhibit or reduce alpha-fetoprotein (AFP) and β2M (β-2-microglobulin) interactions in a disease or disorder associated with AFP-mediated immunosuppression comprising administering a therapeutically effective amount of any of the pharmaceutical composition comprising an inhibitor of AFP-β2M interactions and a pharmaceutically acceptable carrier described herein to a subject in need thereof.

Provided herein, in some aspects, are methods to inhibit or reduce alpha-fetoprotein (AFP) and MHC Class I-related interactions in a disease or disorder associated with AFP-mediated immunosuppression comprising administering a therapeutically effective amount of any of the pharmaceutical compositions comprising an inhibitor of AFP-MHC Class I-related interactions described herein to a subject in need thereof.

In some embodiments of these aspects and all such aspects described herein, the subject has or has been diagnosed with cancer.

In some embodiments of these aspects and all such aspects described herein, the methods further comprise administering an anti-cancer therapy or agent to the subject.

In some embodiments of these aspects and all such aspects described herein, the methods further comprise administering a tumor or cancer antigen.

In some embodiments of these aspects and all such aspects described herein, the subject has or has been diagnosed with a chronic immune infection.

Provided herein, in some aspects, are methods to increase or potentiate alpha-fetoprotein (AFP) and β2M (β-2-microglobulin) interactions in diseases or disorders associated with decreased AFP levels or where increasing AFP levels is beneficial comprising administering a therapeutically effective amount of any of the pharmaceutical composition comprising a potentiator of AFP-β2M interactions described herein to a subject in need thereof.

Provided herein, in some aspects, are methods to increase or potentiate alpha-fetoprotein (AFP) and MHC Class I-related interactions in diseases or disorders associated with decreased AFP levels or where increasing AFP levels is beneficial comprising administering a therapeutically effective amount of any of the pharmaceutical composition comprising a potentiator of AFP-MHC Class I-related interactions described herein to a subject in need thereof.

In some embodiments of these aspects and all such aspects described herein, the subject has or has been diagnosed with an autoimmune disease or disorder.

In some embodiments of these aspects and all such aspects described herein, the subject has or has been diagnosed with host versus graft disease (HVGD), is an organ or tissue transplant recipient, or a recipient of an allogenic transplant.

In some embodiments of these aspects and all such aspects described herein, the subject has had an organ transplantation, partial resection of an organ or other organ injury and is in need of enhanced organ regeneration.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or antigen-binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The terms “antibody fragment” or “antigen-binding fragment” include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}—C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, an “epitope” can be formed both from contiguous amino acids, or noncontiguous amino acids juxtaposed by tertiary folding of a protein or by binding of one to another polypeptide. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

The term “therapeutically effective amount” therefore refers to an amount of the inhibitors or potentiators described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign tumors and malignant cancers, as well as dormant tumors or micrometastases. Cancers which migrate from their original location and seed vital organs can eventually lead to the death of the subject through the functional deterioration of the affected organs. Hematopoietic cancers, such as leukemia, are able to out-compete the normal hematopoietic compartments in a subject, thereby leading to hemopoietic failure (in the form of anemia, thrombocytopenia and neutropenia) ultimately causing death.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

As used herein, an “autoimmune disease” refers to a class of diseases in which a subject's own antibodies react with host tissue or in which immune effector T cells are autoreactive to endogenous self-peptides and cause inflammation and/or destruction of tissue. Thus an immune response is mounted against a subject's own antigens, referred to as self-antigens. A “self-antigen” as used herein refers to an antigen of a normal host tissue. Normal host tissue does not include cancer cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts AFP superimposed on human serum albumin (HSA) crystal structure (PDB ID: 4N0F) and the resulting AFP model.

FIG. 2 depicts a model of AFP:FcRn:β2M ternary complex based on HSA:FcRn:β2M crystal structure (PDB ID: 4N0F).

FIG. 3 demonstrates that AFP S527/D528 residues make contacts with β2M E50 and Y67 that are not present in HSA (N503, A504) thereby demonstrating new interactions. HSA/AFP non-conserved residues are shown that increase AFP binding to FcRn through new contacts with β2M.

FIG. 4 demonstrates that AFP R604 makes additional contacts with carbonyl oxygen E50 of β2M, thereby providing new interactions. HSA/AFP non-conserved residues are shown that increase AFP binding to β2M. HSA Q580 lacks these interactions.

FIG. 5 depicts a model of AFP:β2M binary complex, illustrating that AFP can associate with β2M alone, i.e., without association with MHC Class I-related family of molecules.

FIG. 6 depicts Tables 6a) and 6b) based on binary complex model illustrating four distinct interfaces of contact between AFP and β2M.

FIG. 7 shows that human (h) AFP binds to hβ2M alone at pH 6. 100 RU of hβ2M were immobilized on the surface of an SM5 chip by amine coupling and hAFP was injected at different concentrations (2000-15.6 nM AFP).

FIG. 8 shows that hβ2M binds to hAFP at pH 6. 200 RU of hAFP were immobilized on the surface of an SM5 chip by amine coupling and hβ2M was injected at different concentrations (10,000-156 nM).

FIG. 9 shows summary of binding kinetics between human or mouse β2M and AFP of human (cord blood or recombinant) or mouse origin.

FIG. 10 shows amino acid sequence comparisons between human FcRn and representative human MHC class I molecules with residues shaded in gray indicating amino acids in FcRn responsible for interactions with HSA (MR1: AAC72900.1; FcRn: NP_004098.1; HLA-A: BAA07530.1; HLA-B: P30483.1; HLA-C: NP_001229971.1; HLA-G: NP_002118.1; HFE: AIS82633.1; HLA-E: AAA52655.1; HLA-F: BAB63337.1; ZA2G: NP_001176.1; CD1D: NP_001757.1; CD1A: NP_001754.2; CD1B: P29016.1; CD1C: P29017.2; CD1E: P15812).

FIG. 11A depicts a heat map and percent amino acid identity between different members of the MHC class I family. FIG. 11B depicts a Phylogenetic Tree (Cladogram) of different members of the MHC class I family.

FIG. 12 depicts a table with Root mean square deviation (RMSD) between FcRn and different MHC class I molecules and indicates structural similarity between FcRn and MHC class I family members. Crystal structures of FcRn and different MHC class I family members were superimposed with PYMOL to calculate RMSD and structural similarity. PDB ID Numbers are shown in parentheses.

FIG. 13 depicts a table with predicted surface area of interaction between AFP and particular members of MHC class I family and free energy of their binding based on superposition of crystal structure of FcRn and different MHC class I-related family members.

FIG. 14 depicts a table illustrating two binding interfaces with relative close conservation between MHC Class I-related family members that are predicted to form contacts with AFP. Crystal structures were retrieved from Protein Data Bank.

FIG. 15 depicts various binding interfaces of AFP that are predicted to form contacts with MHC Class I-related family members.

FIG. 16 depicts a model of AFP superimposition on HLA-A: hβ2M complex.

FIG. 17 depicts superimposition of MR1 on FcRn and a modeling of AFP: MR1:β2M: ternary complex.

FIG. 18 shows that human AFP binds to HLA-A*02:01-β2M at pH 6. 100 RU of human HLA-A were immobilized on the surface of an SM5 chip by amine coupling and human AFP was injected at different concentrations (2000-15.6 nM AFP).

FIGS. 19A-19B demonstrate that hAFP decreases detection of hβ2M on human PBMCs.

PBMC were incubated with different concentrations of hAFP or HSA (0, 10, 100 or 200 μg/ml) for 30 minutes in serum free media at (FIG. 19A) pH 7.4 or (FIG. 19B) pH 6, followed by 20 min staining with anti hβ2M antibody at corresponding pH. The cells when then fixed in 2% paraformaldehyde and analyzed on MACSQuant. Individual Representative histograms (right panels) and Mean Fluorescence Intensity (MFI) bar graphs (left panels) are shown.

FIGS. 20A-20B demonstrate that hAFP decreases detection of hβ2M on hβ2M transgenic (Tg) mouse Dendritic cells. Dendritic cells obtained from hFcRn and hβ2M transgenic mice were incubated with different concentrations of hAFP or HSA (0, 10, 100 or 200 μg/ml) for 30 minutes in serum free media at (FIG. 20A) pH 7.4 or (FIG. 20B) pH 6, followed by 20 min staining with anti hβ2M antibody at the corresponding pH. The cells when then fixed in 2% paraformaldehyde and analyzed on MACSQuant. Individual Representative histograms (right panels) and Mean Fluorescence Intensity (MFI) bar graphs (left panels) are shown.

FIG. 21 depicts how an elaborate “handshake” is performed between a cytotoxic CD8⁺ T-cell and most nucleated cells in the body that express MHC-Class I constitutively. The primary contact is between the T-cell receptor (TCR: purple and blue colour) on the surface of T cell and MHC class I: NM-peptide complex (orange, pink and bright red here, respectively). Then, CD8 co-receptor (light and dark green) binds to other portions of the MHC class I, strengthening the interaction. This illustration was adapted from Protein Data Bank and created using several PDB entries 1AKJ, 1BD2, 1FYT, 1M4, and 1WIO.

FIG. 22 depicts a model of AFP superimposition on the HLA-A2:β2M and CD8 co-receptor complex (PDB ID: 1AKJ) and illustrates that AFP binding does not affect CD8 co-receptor binding to MHC class I molecules.

FIG. 23 depicts a model of HLA-A: β2M and T cell receptor (TCR) complex (PDB ID: IBD2) as well as AFP superimposition on this complex, illustrating that binding of AFP would prevent TCR binding to MHC Class I molecules.

FIG. 24 depicts a model of inhibitory human NK cell receptor, KIR2DL2 and HLA-Cw3: hβ2M (PDB ID: 1EFX) as well as AFP superimposition on this complex, illustrating that AFP would not block their interaction.

FIG. 25 depicts a model of AFP superimposed on HLA-E:β2M and inhibitory receptor NKG2A:CD94 complex (PDB ID: 3CDG) and illustrates that the binding of AFP would prevent the binding of NKG2A:CD94 receptor to HLA-E.

FIG. 26 depicts a model of AFP superimposed on HLA-E:β2M and TCR complex (PDB: 2ESV) and illustrates that the binding of AFP to HLA-E would prevent the interaction of TCR with HLA-E.

FIG. 27 depicts a model of AFP superimposed on HLA-G: β2M and LILRB2 complex (PDB ID 2DYP) and illustrates that the binding of AFP to HLA-G would not block the interaction between HLA-G and LILRB2.

FIG. 28 depicts a model of AFP superimposed on CD1D:β2M and NKT15 complex (PDB ID: 2P06) and illustrates that the binding of AFP to CD1D would have little impact on interaction between CD1D and NKT15.

FIG. 29 demonstrates that human AFP derived from cord blood binds to HLA-A*02:01-β2M at pH 6. 140 RU of biotinylated human HLA-A2:01 were immobilized on the surface of a neutravidin chip and human AFP was injected at different concentrations (2000-15.6 nM AFP). The Langmuir 1:1 ligand binding model provided by the BIA evaluation software (version 4.1) was used to determine the binding kinetics. The closeness of the fit is described by the statistical value χ2.

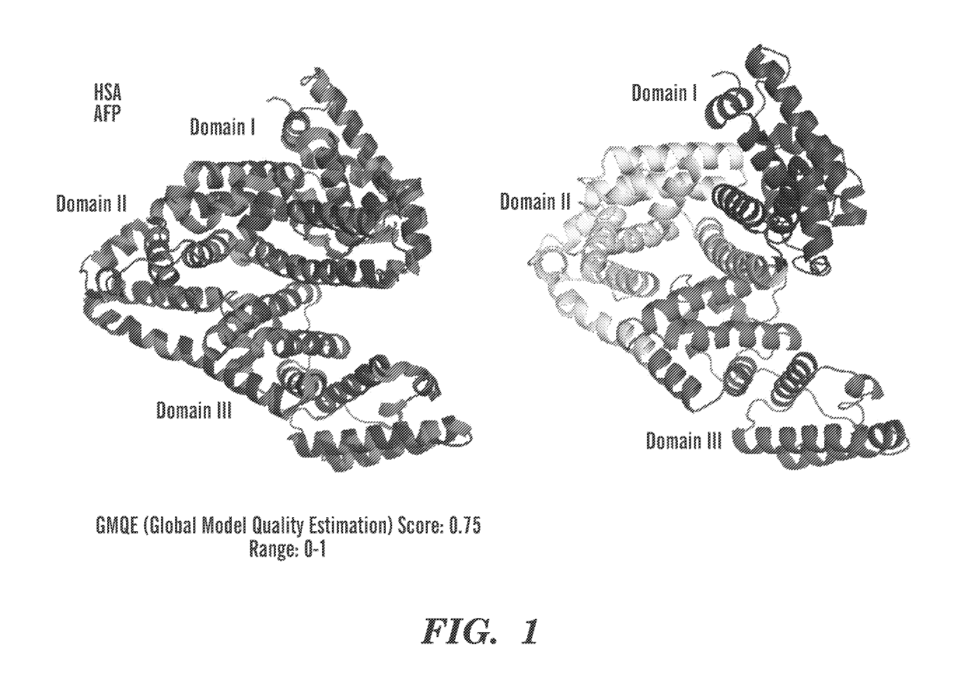

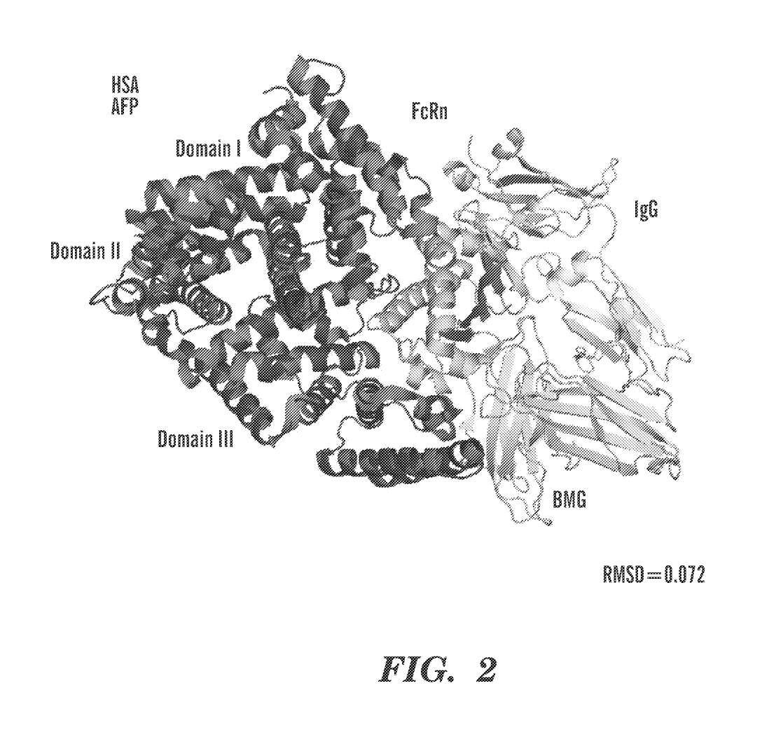

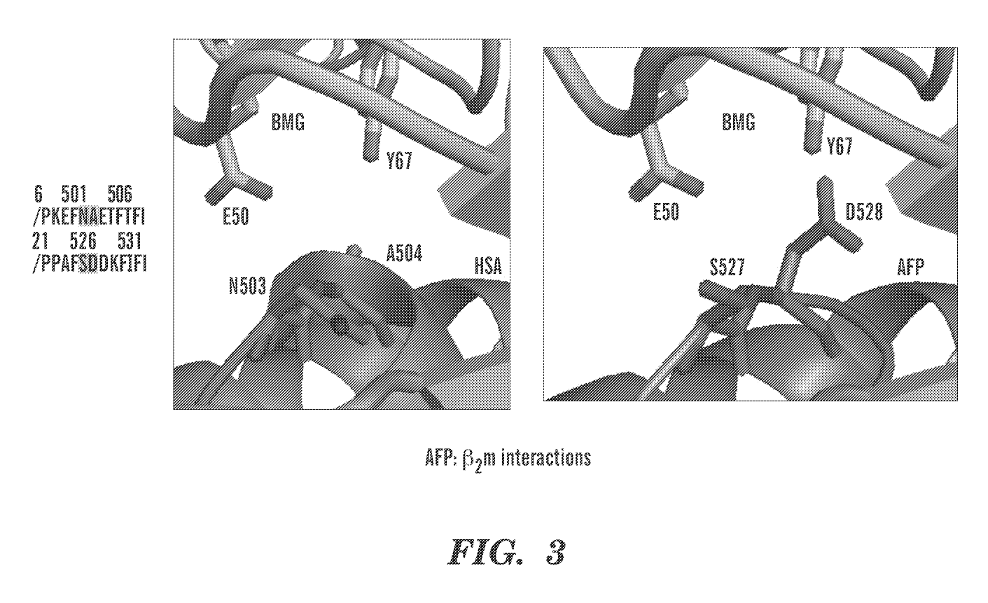

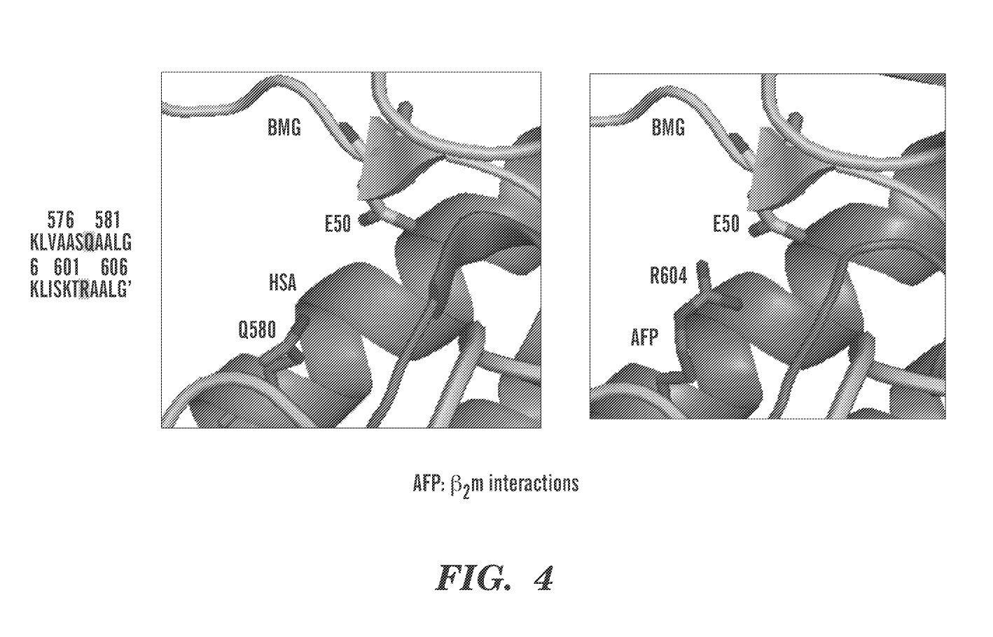

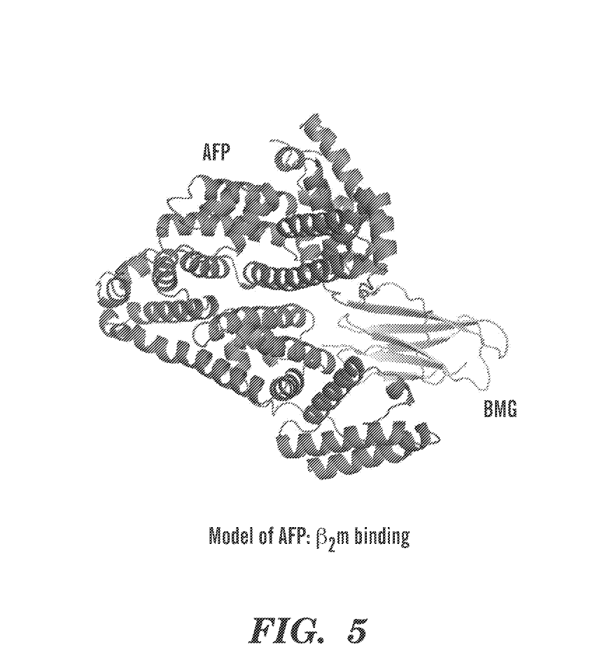

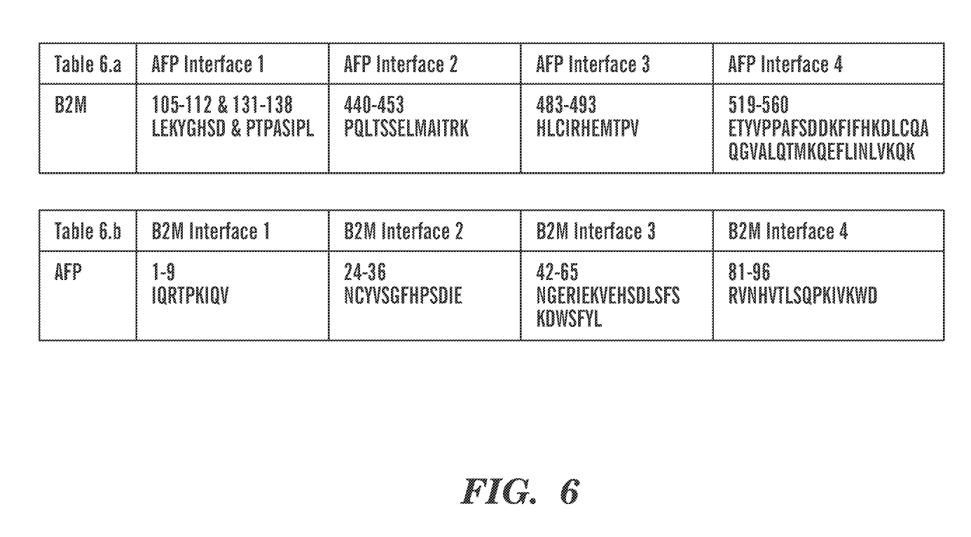

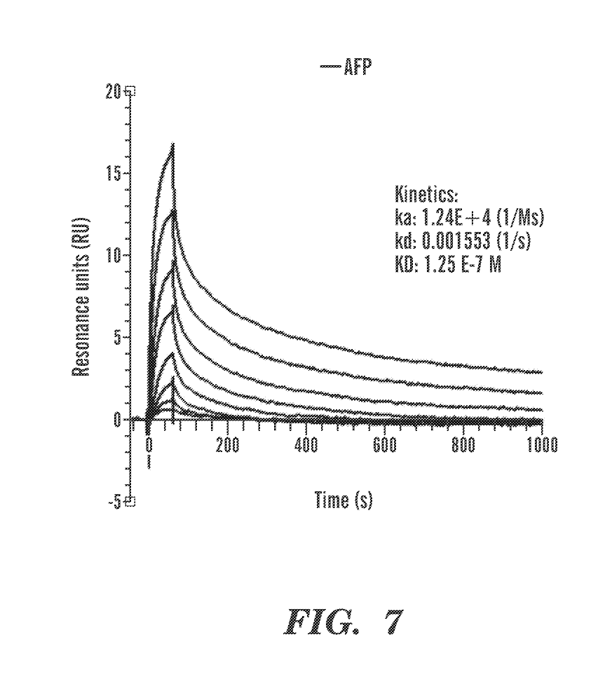

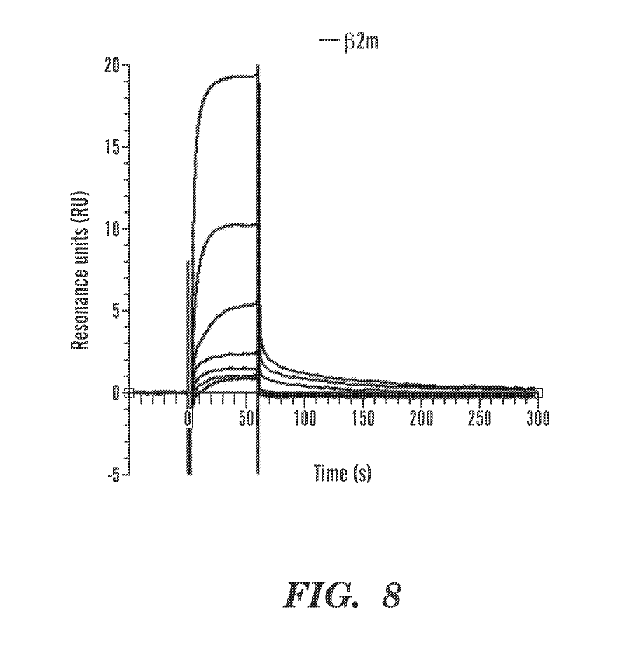

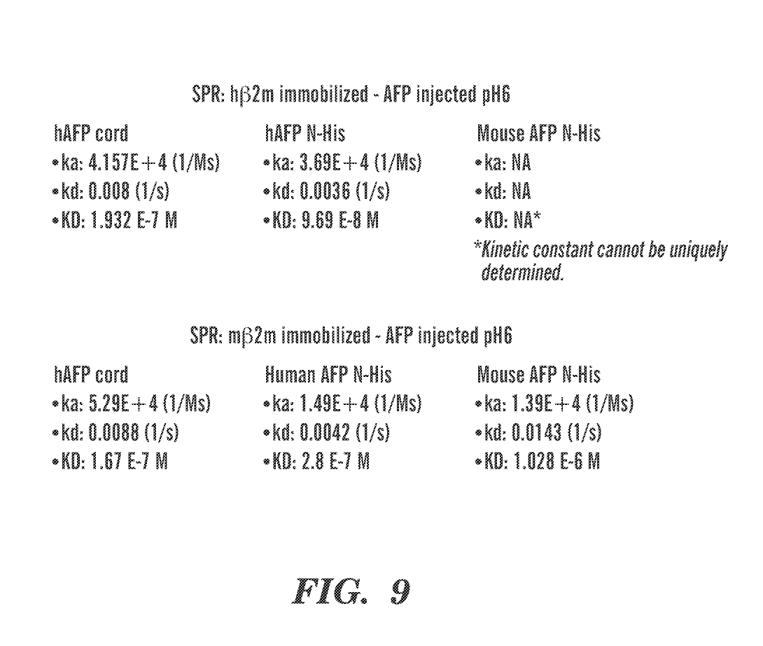

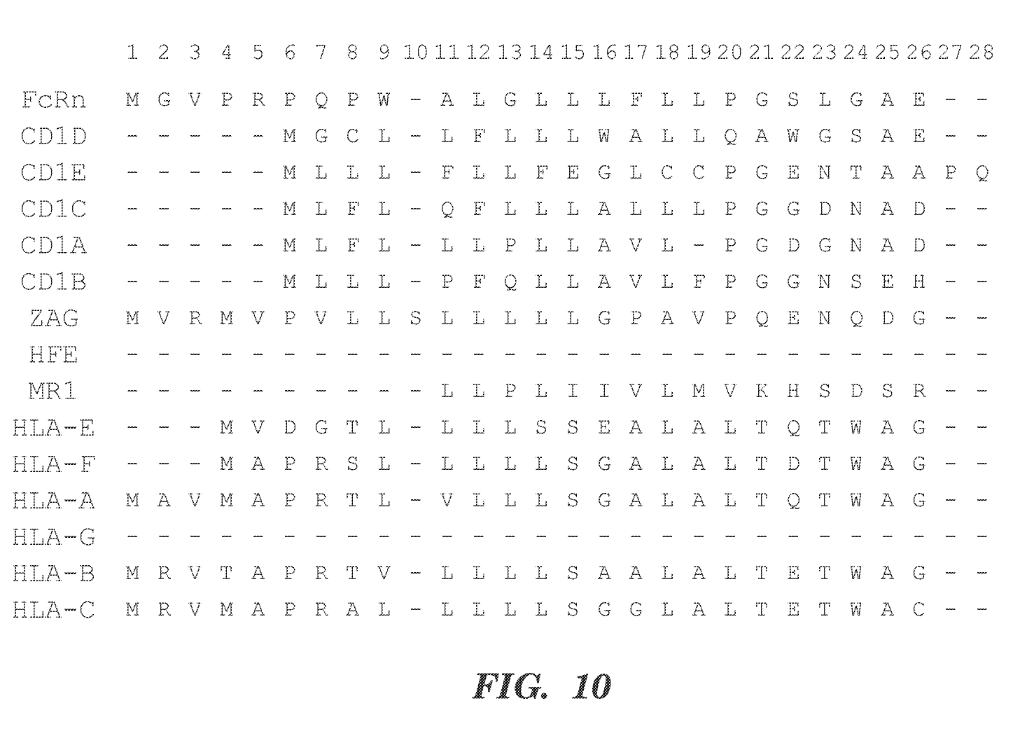

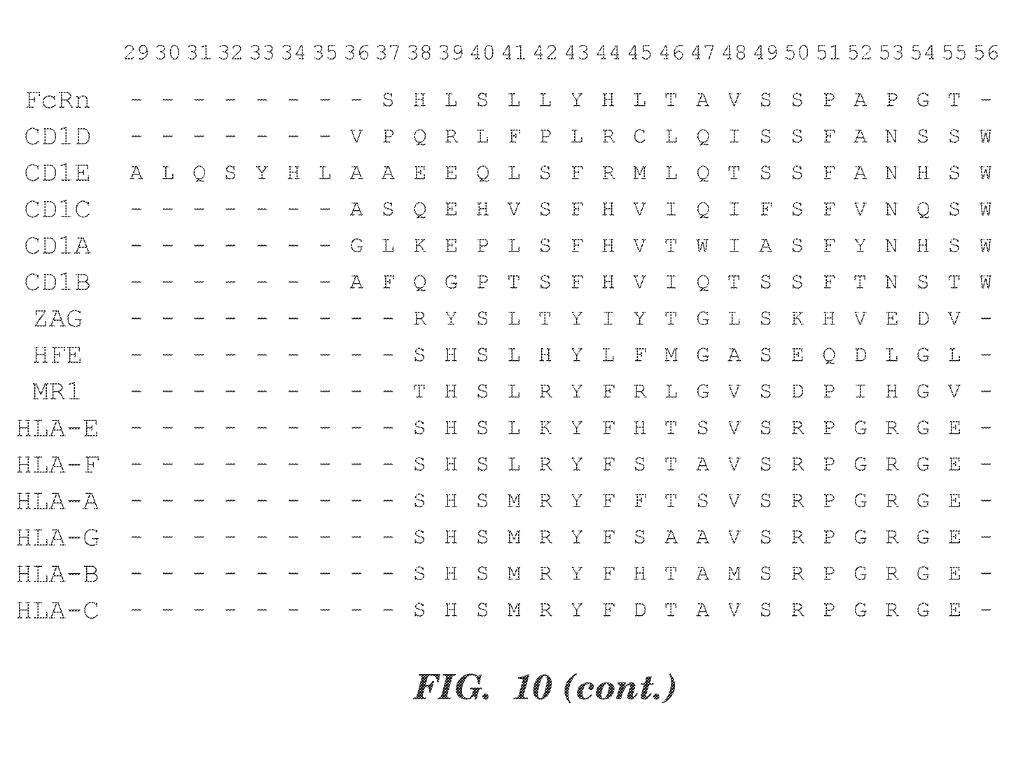

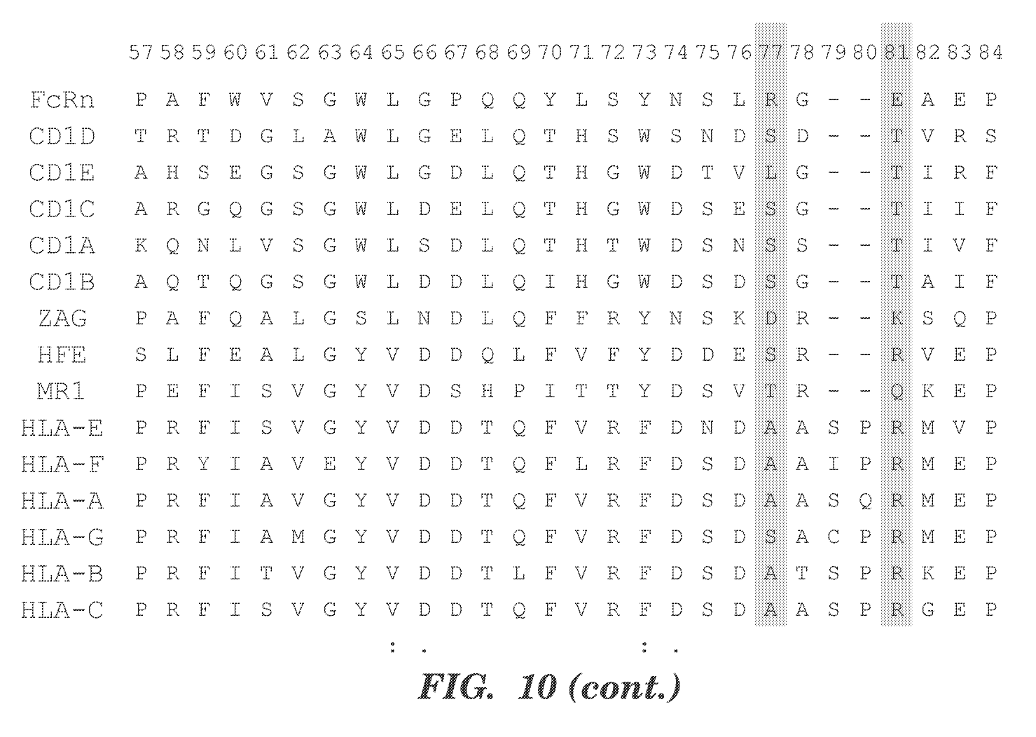

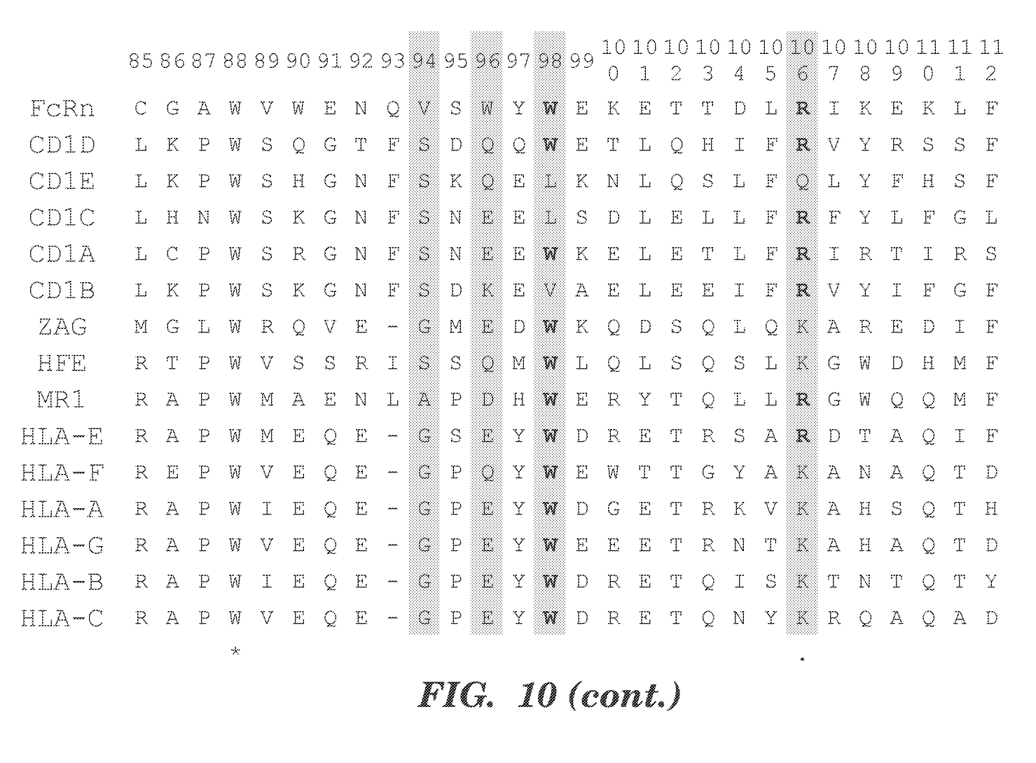

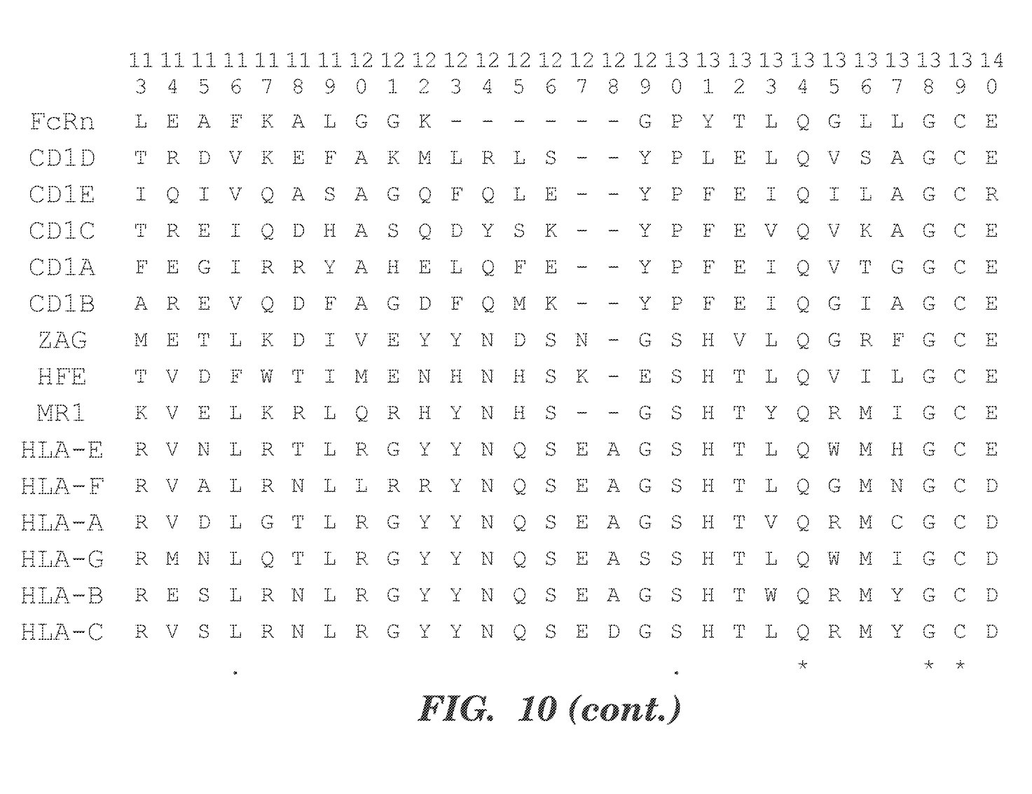

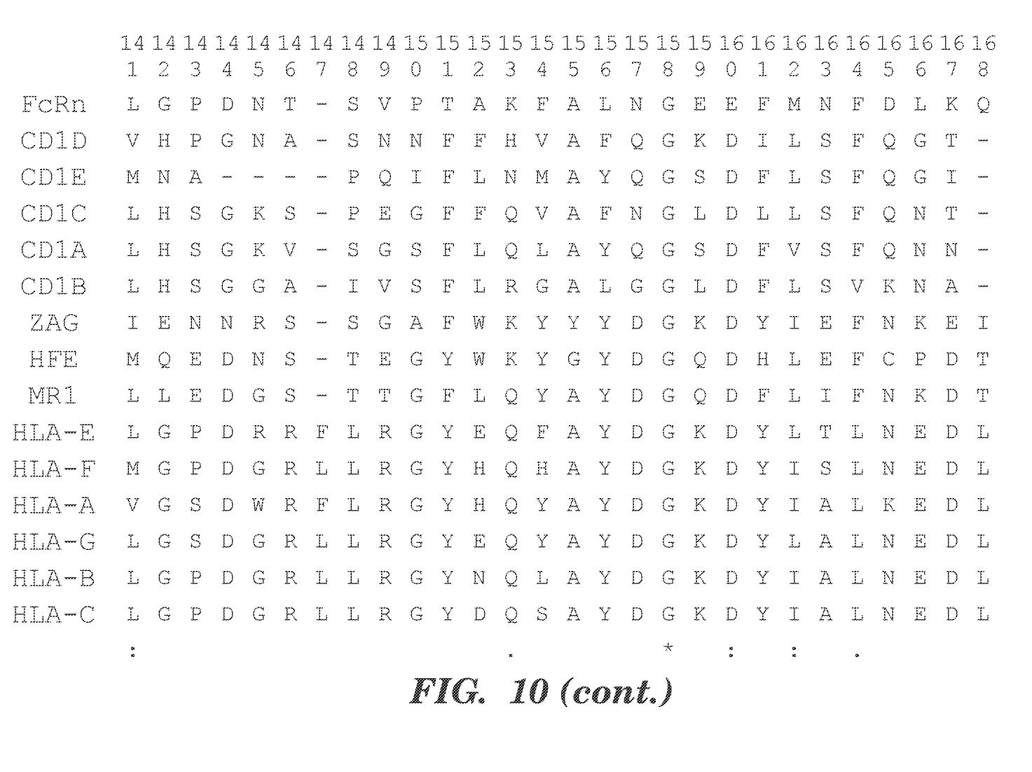

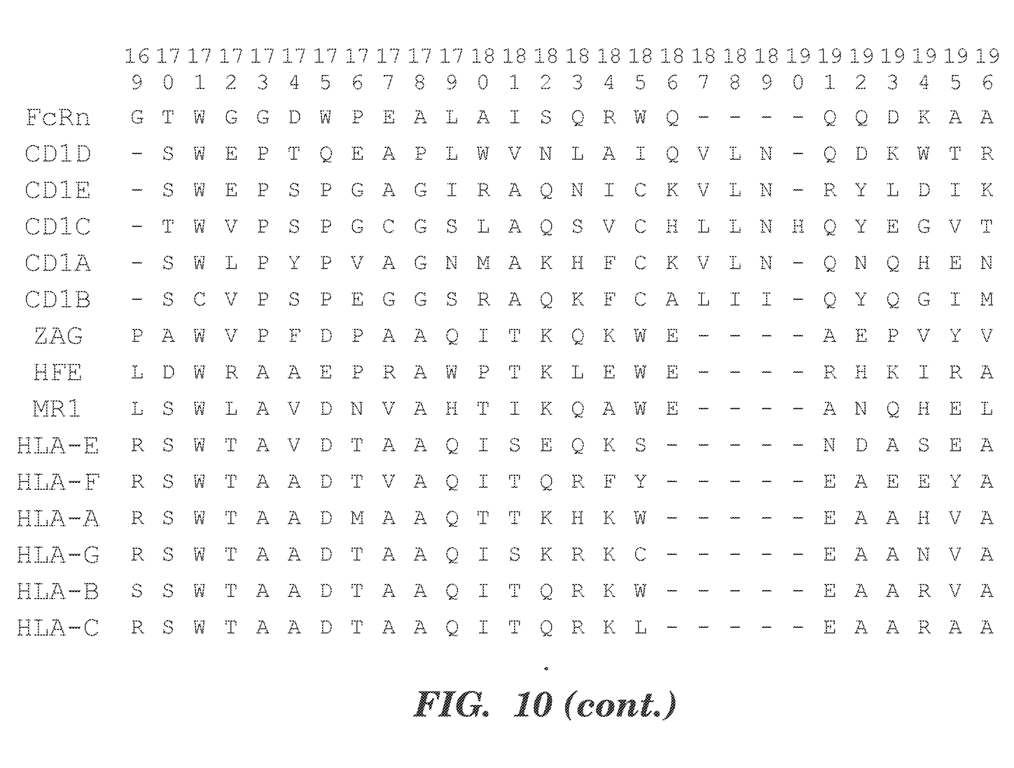

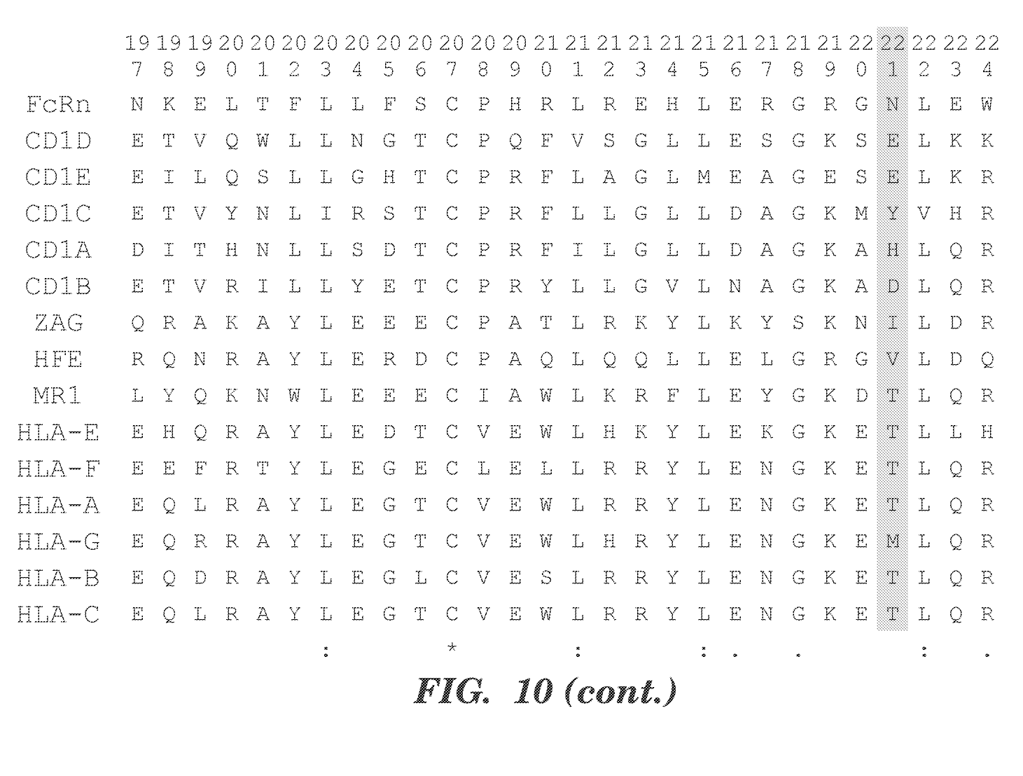

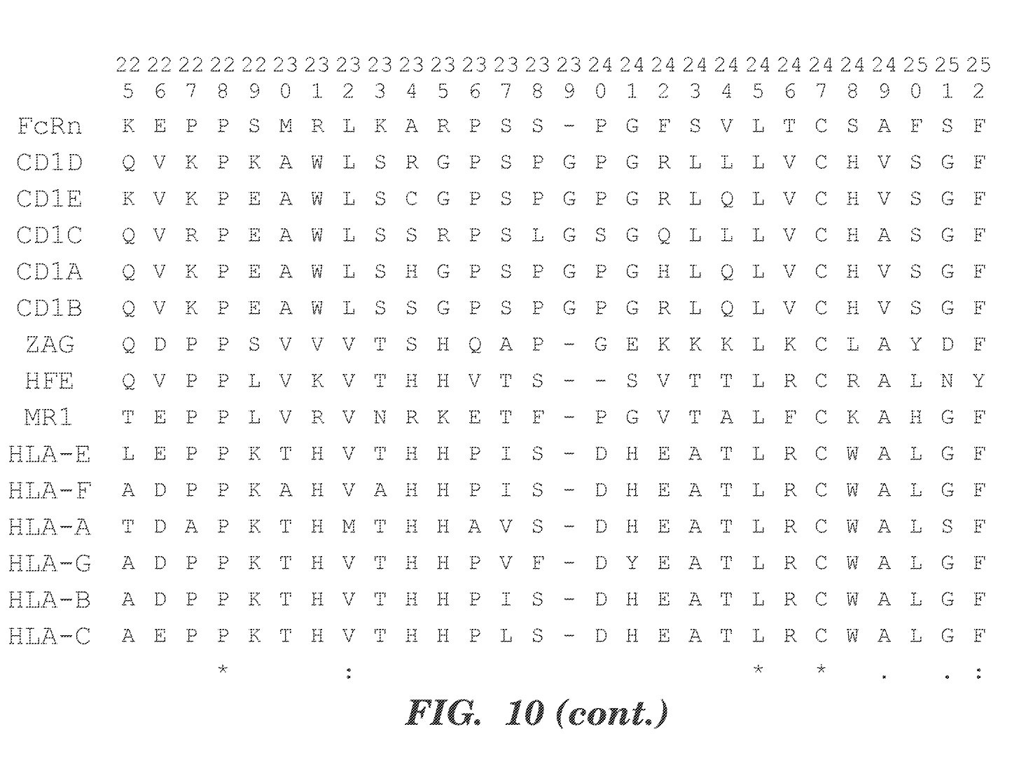

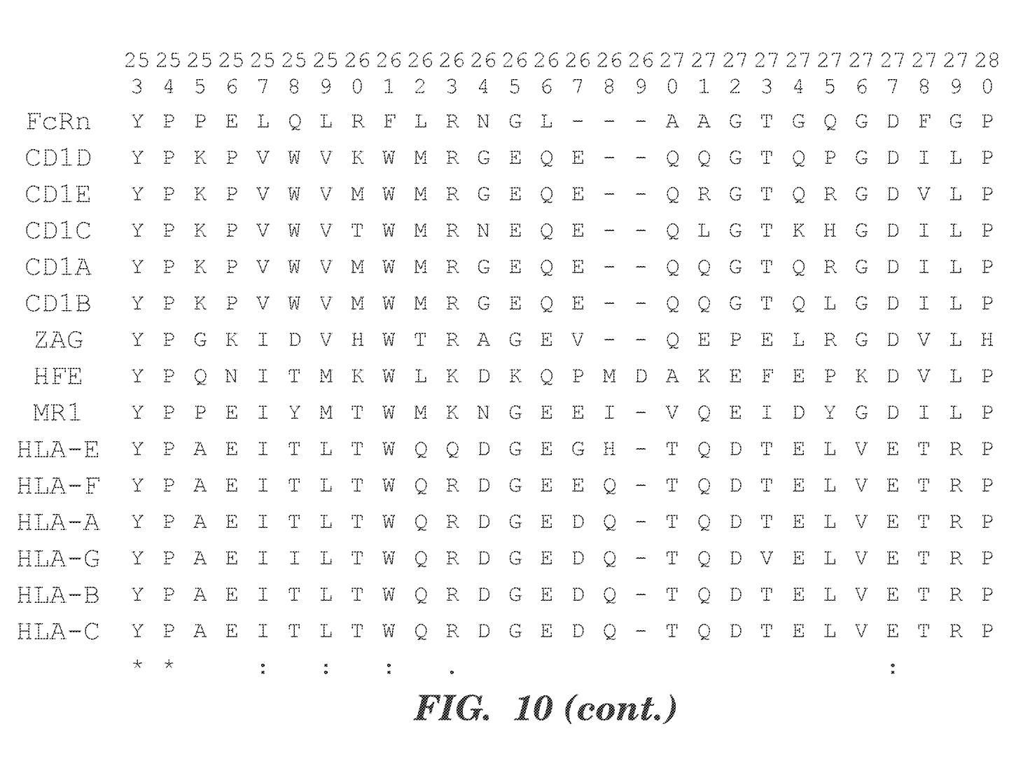

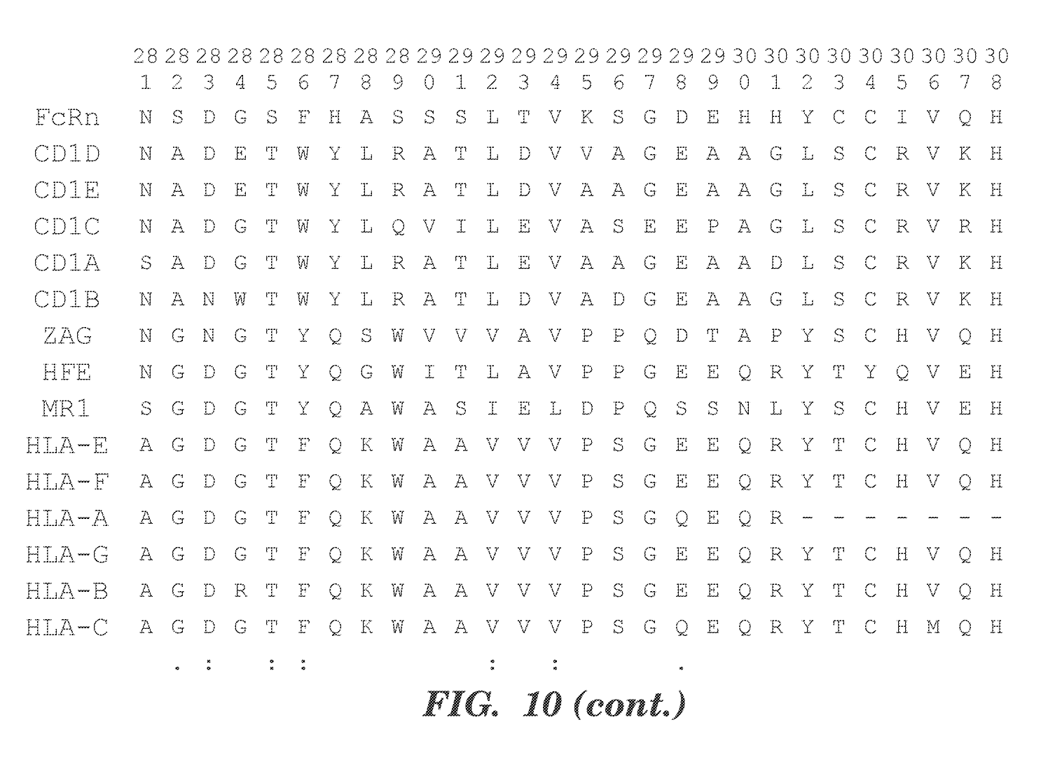

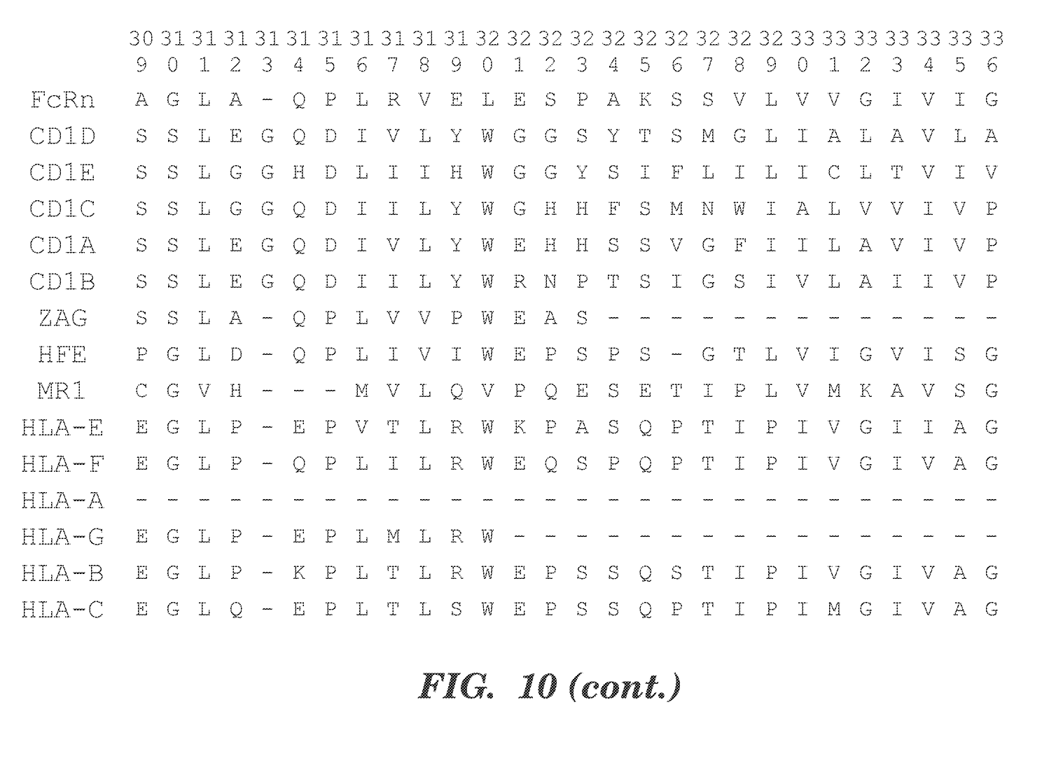

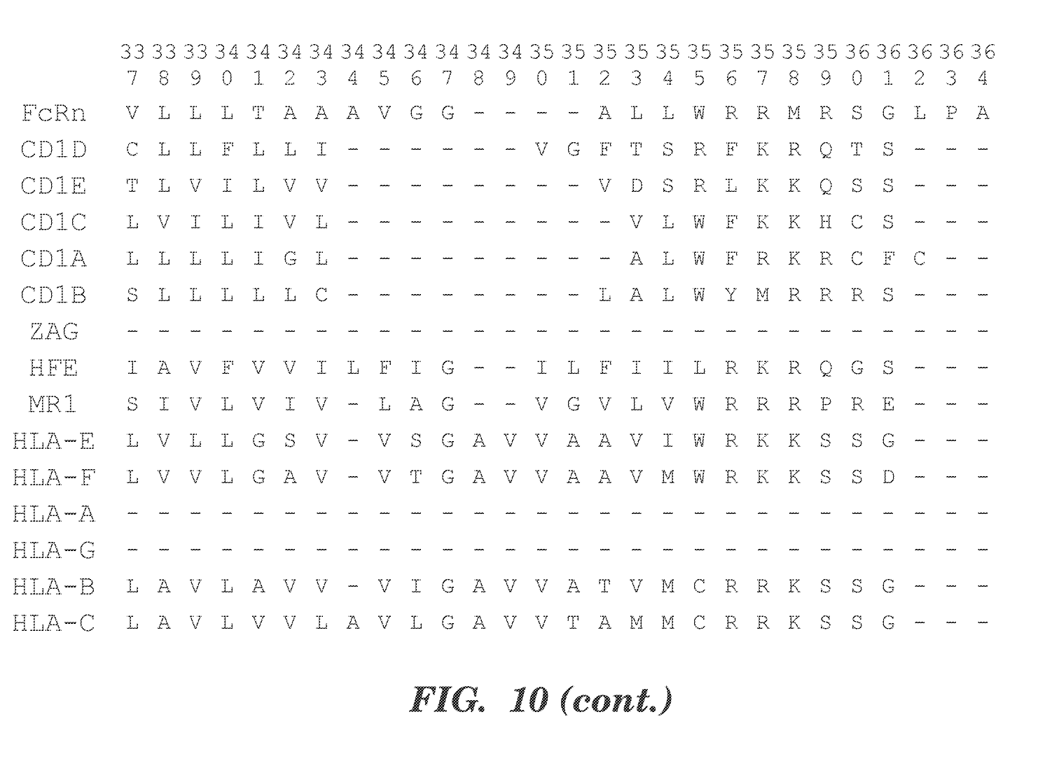

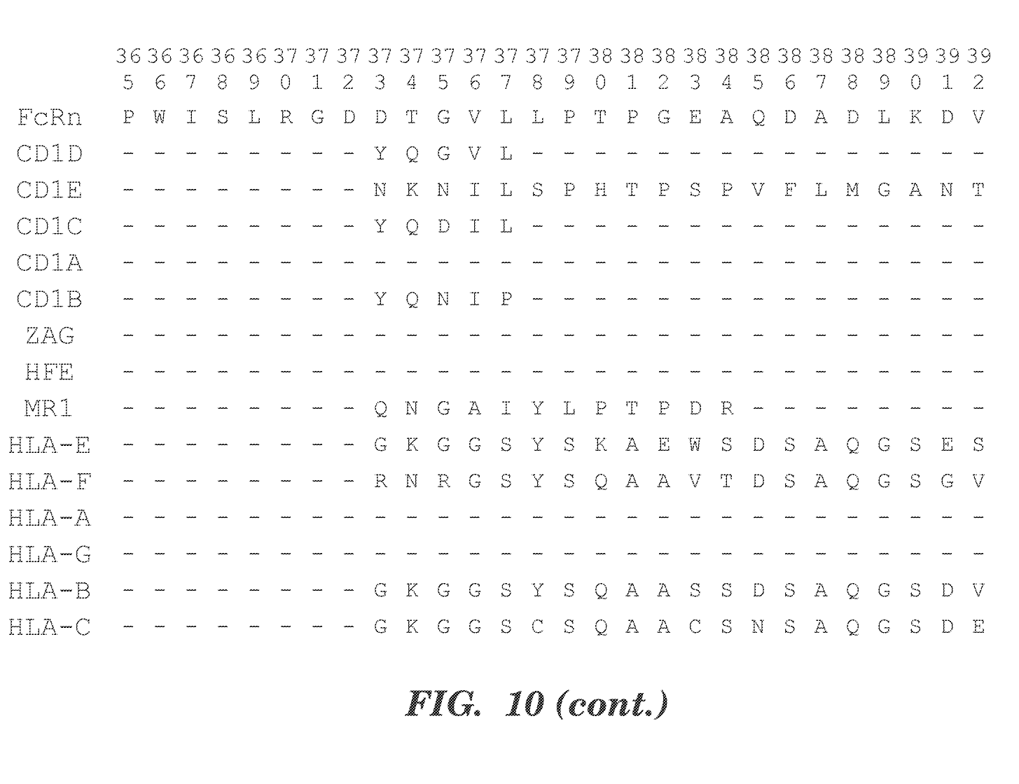

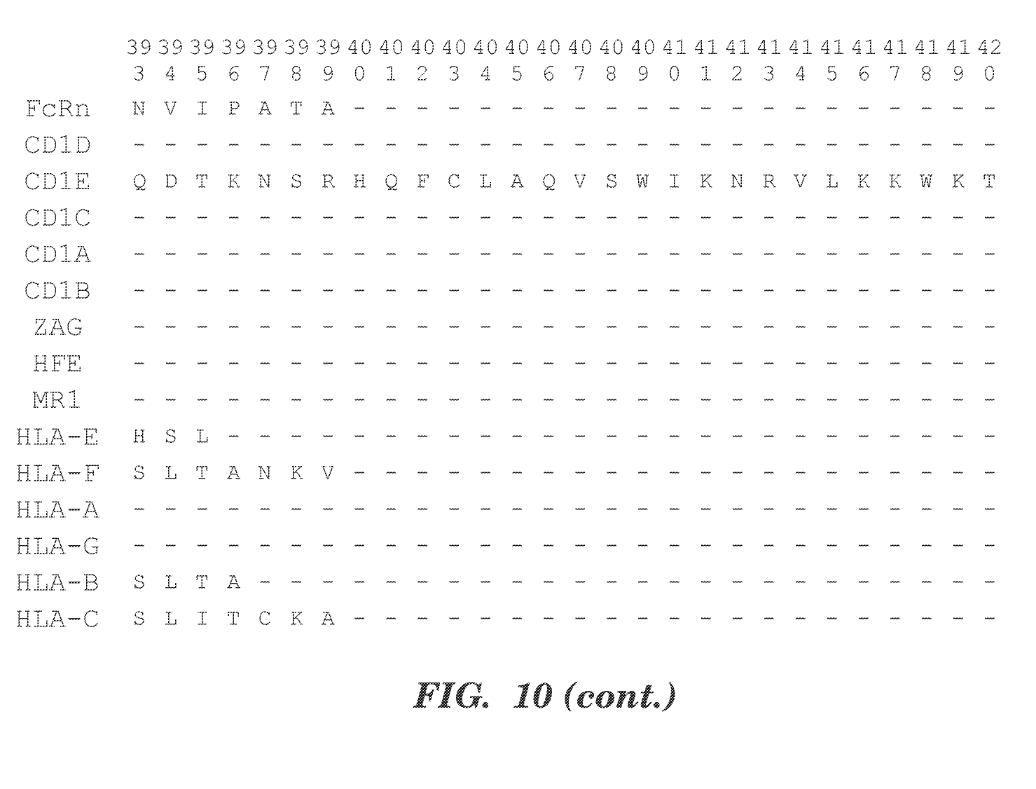

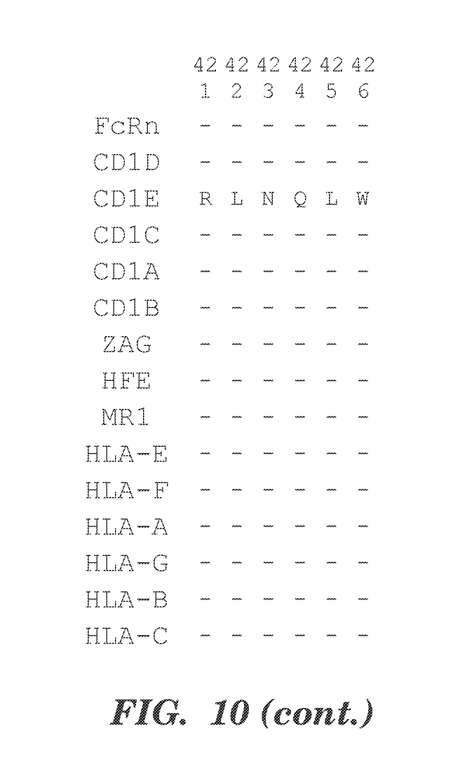

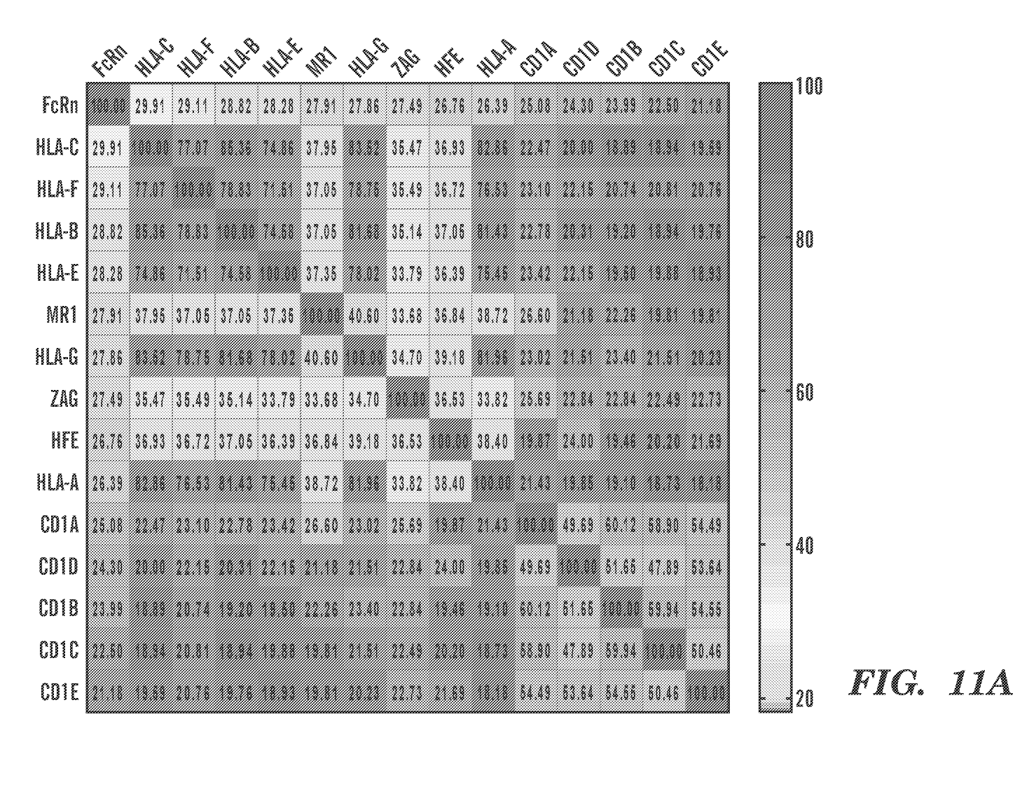

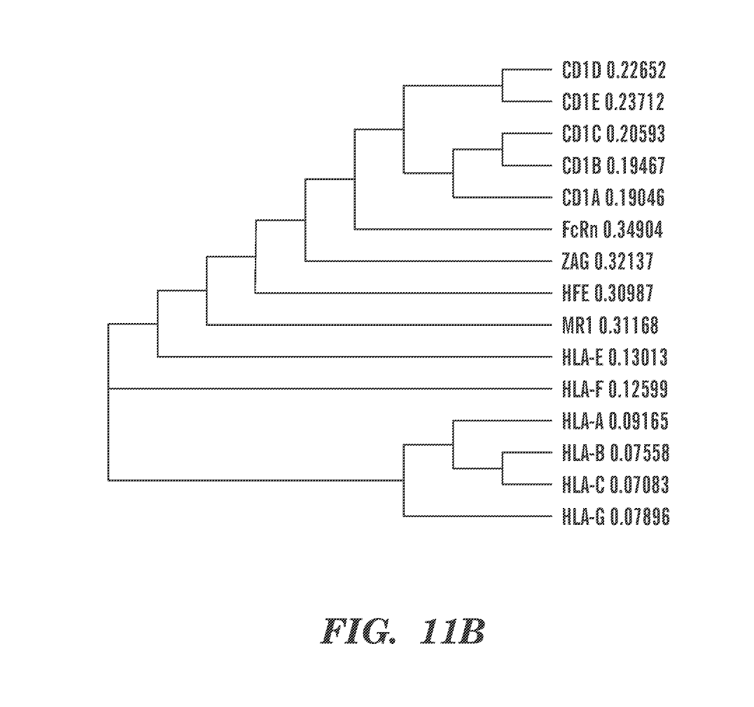

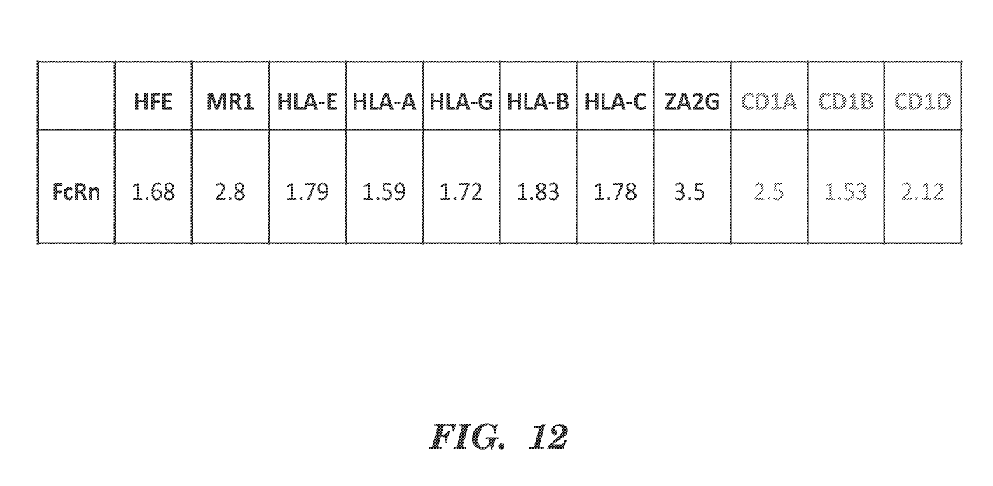

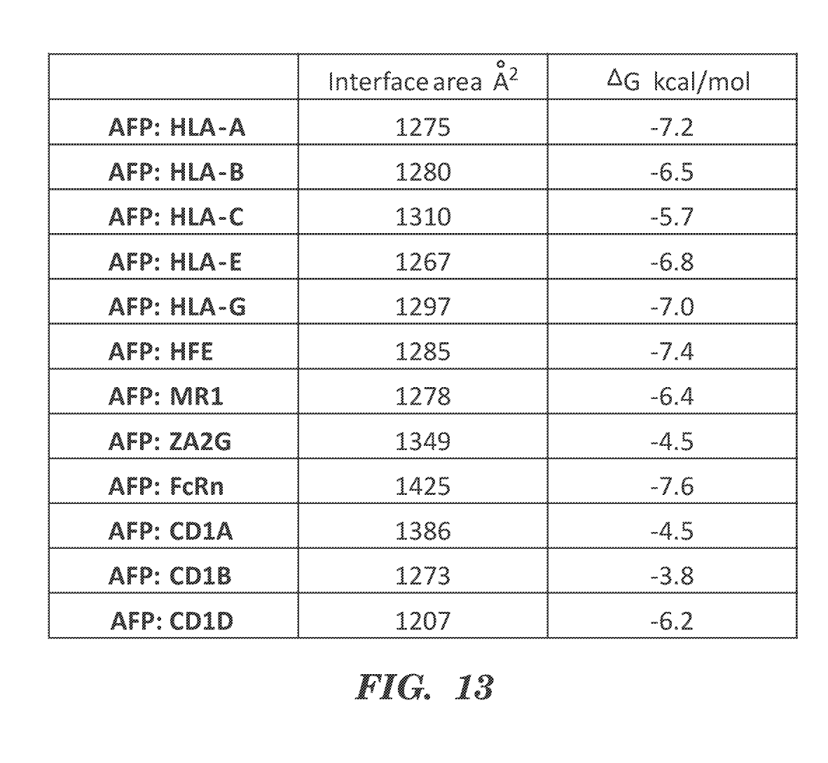

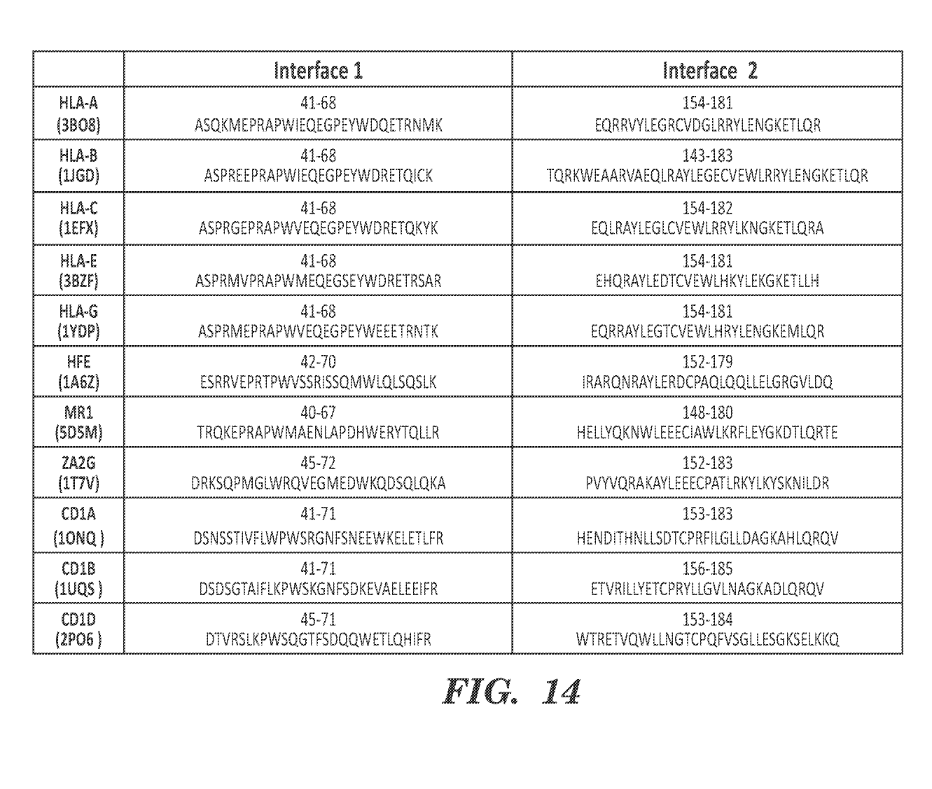

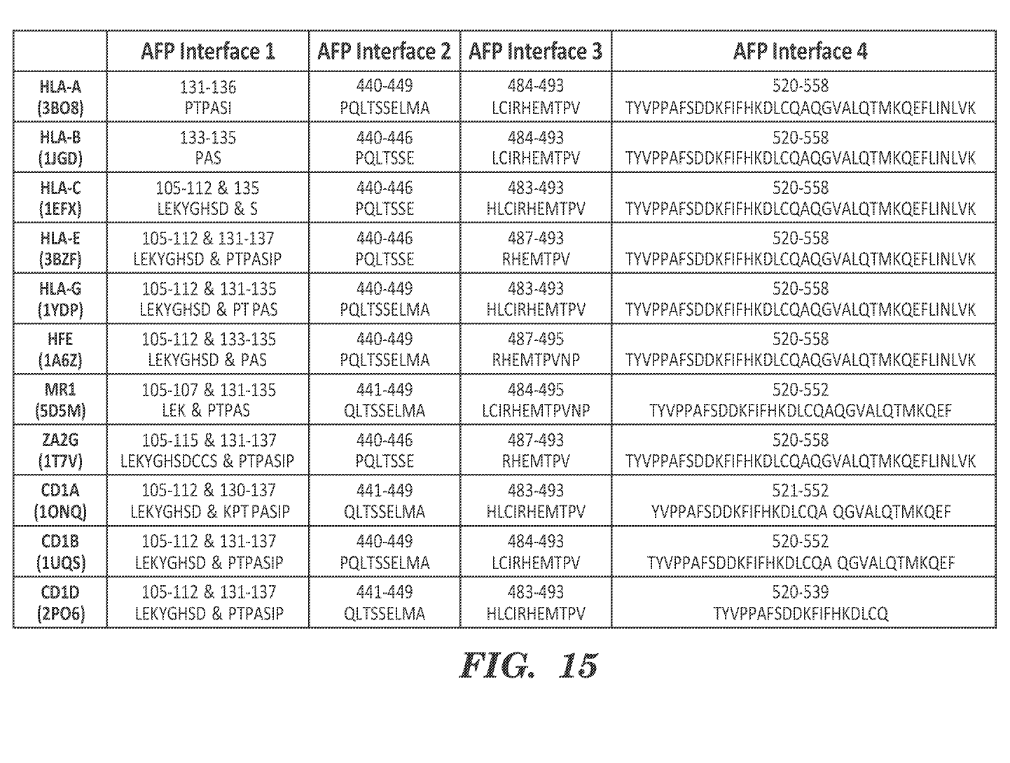

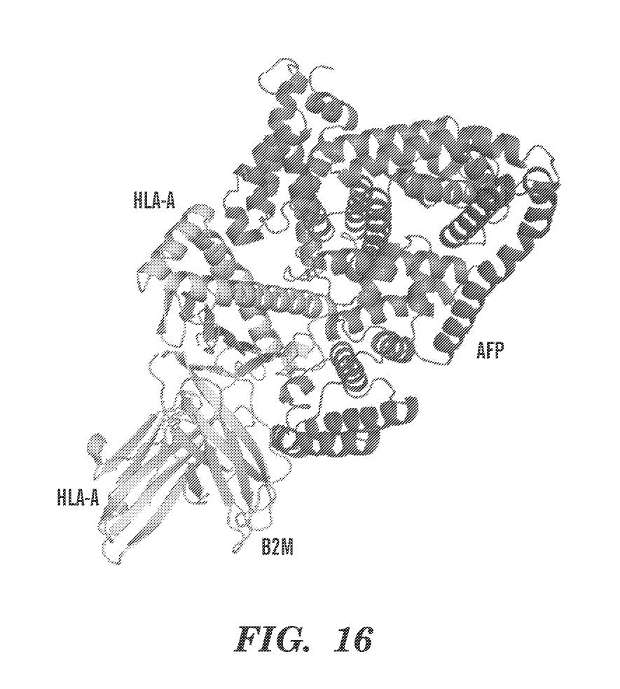

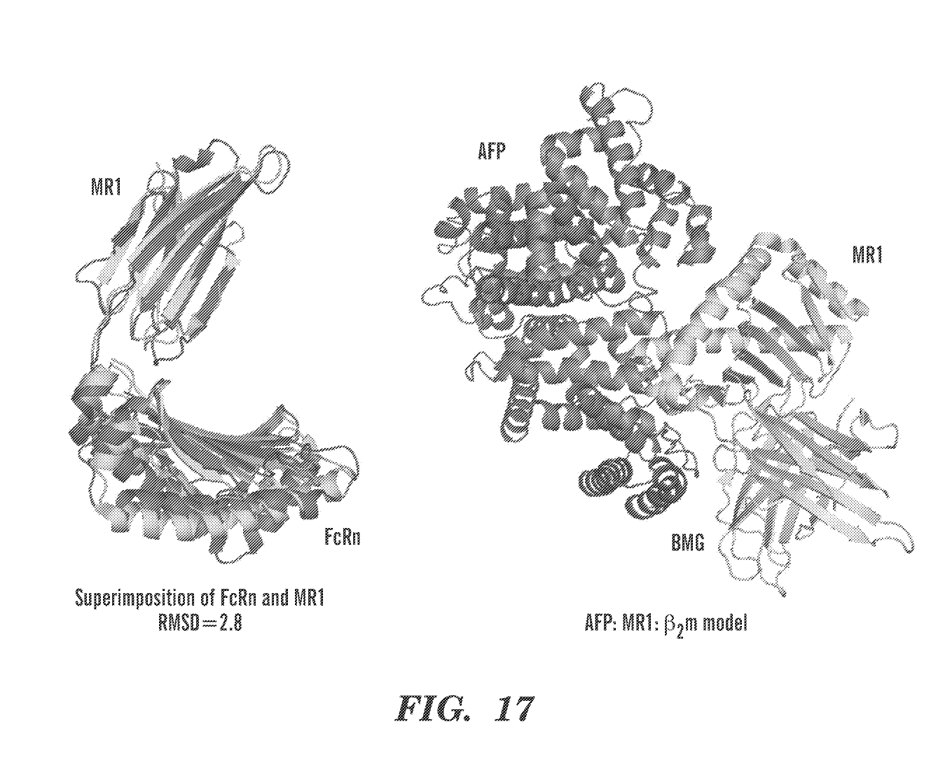

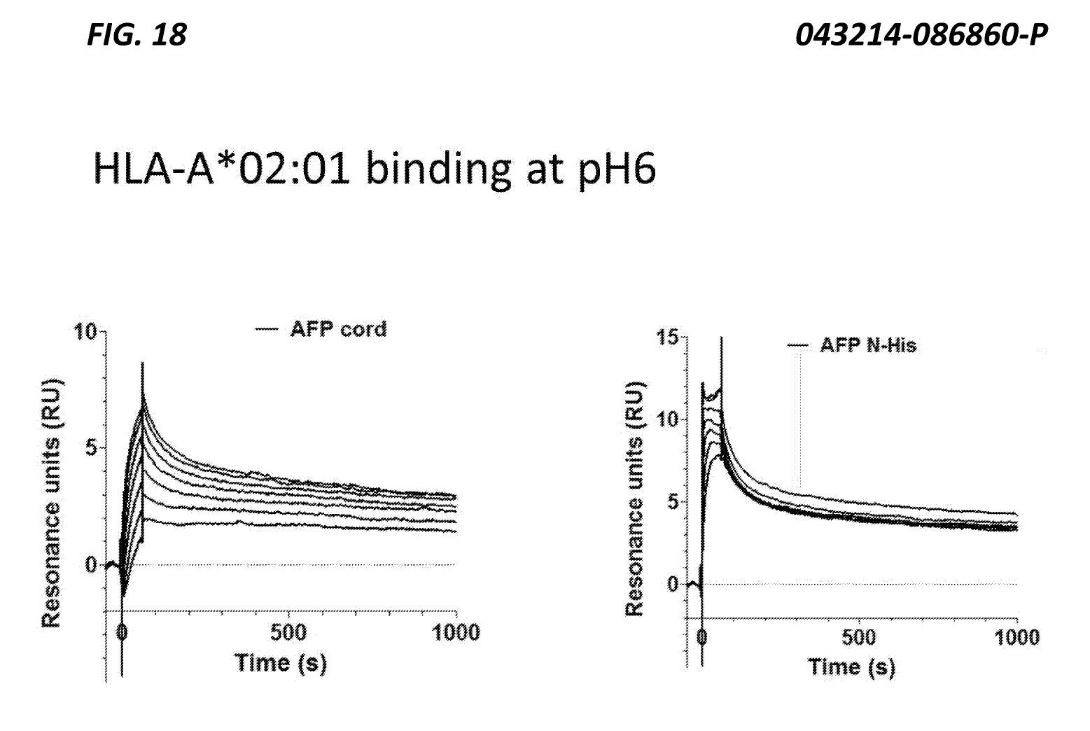

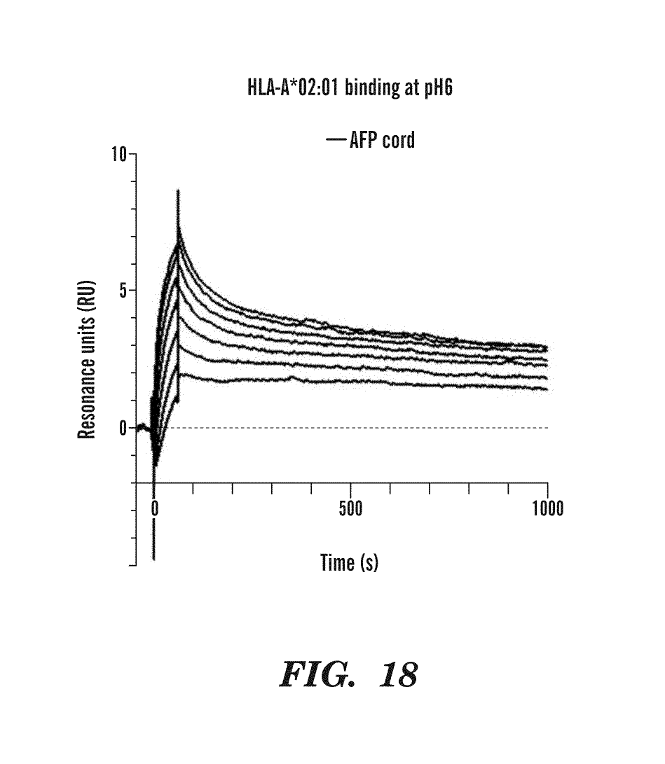

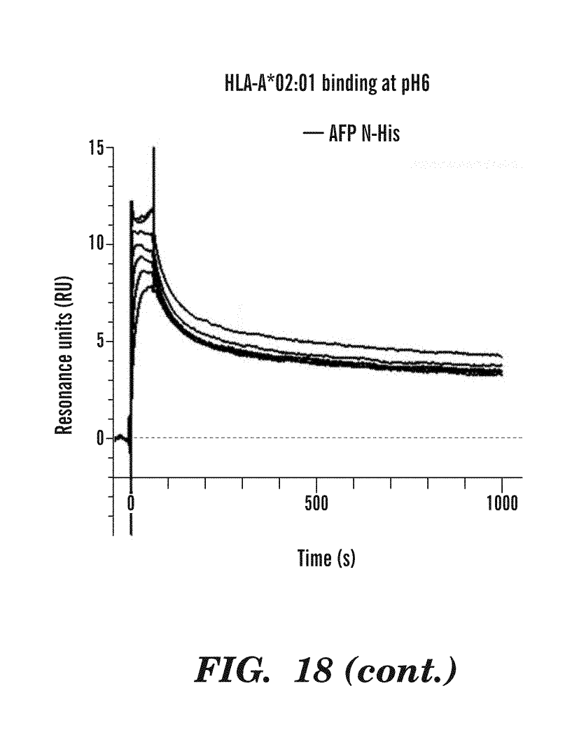

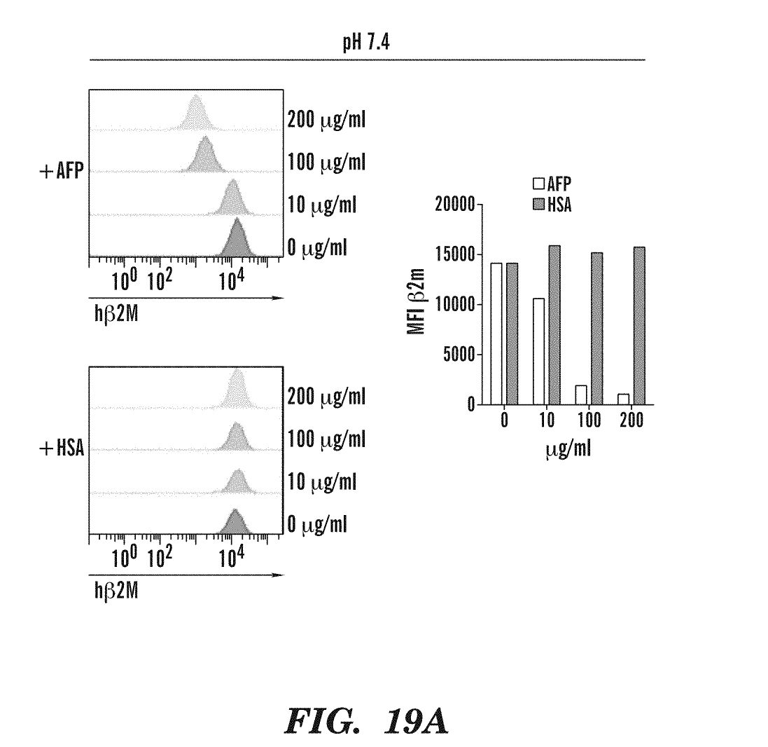

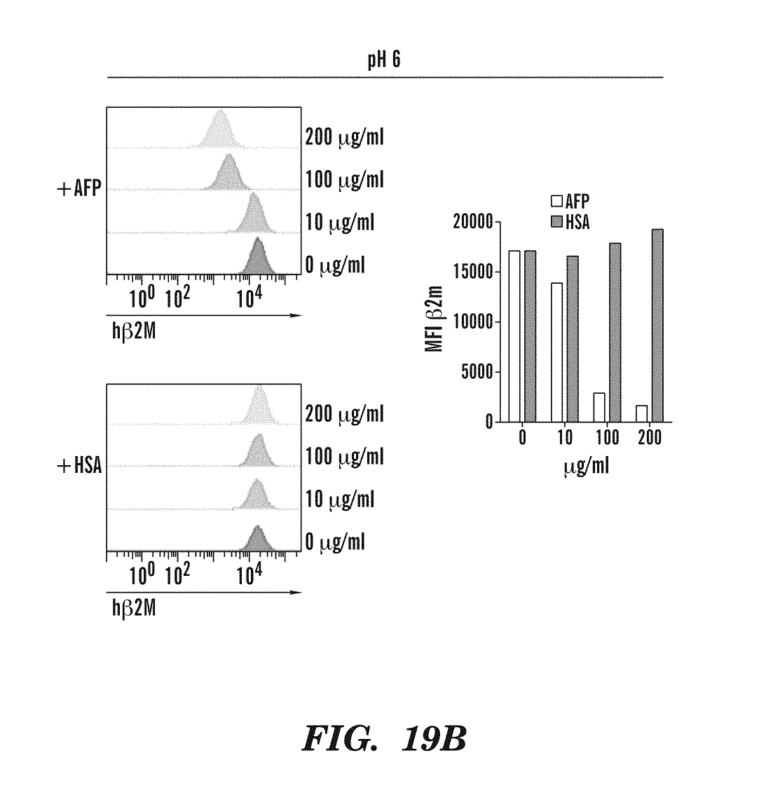

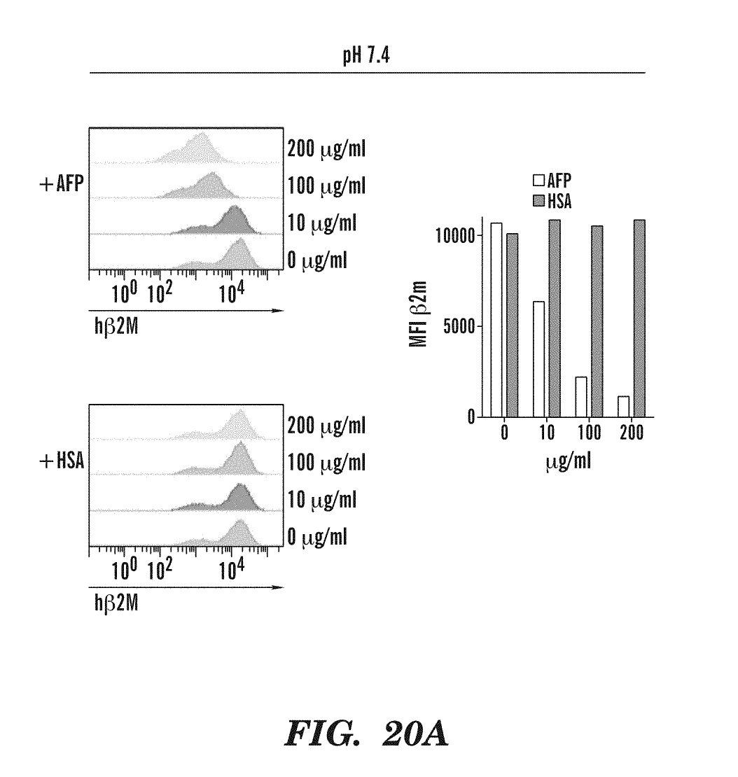

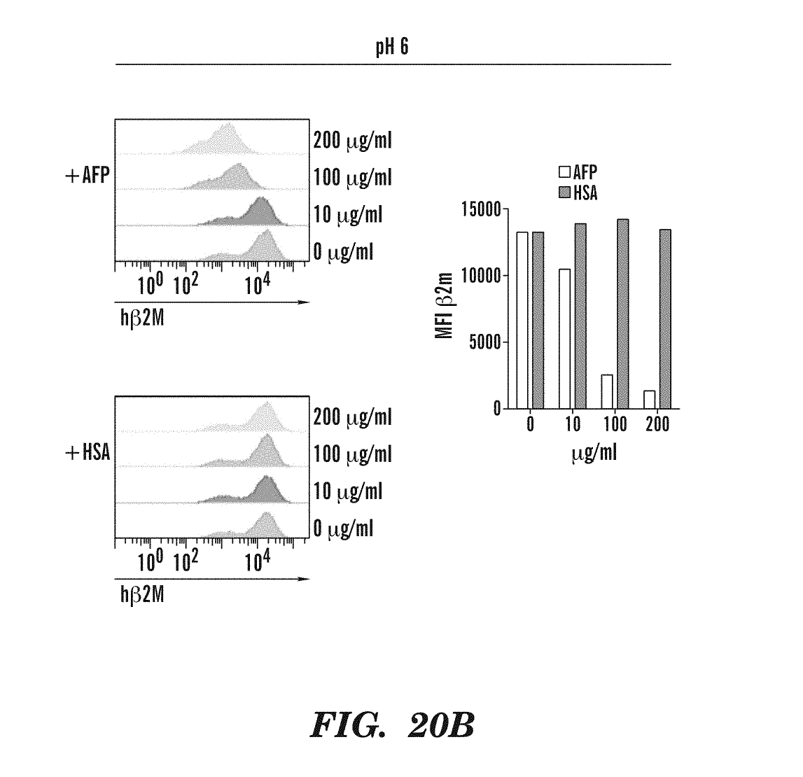

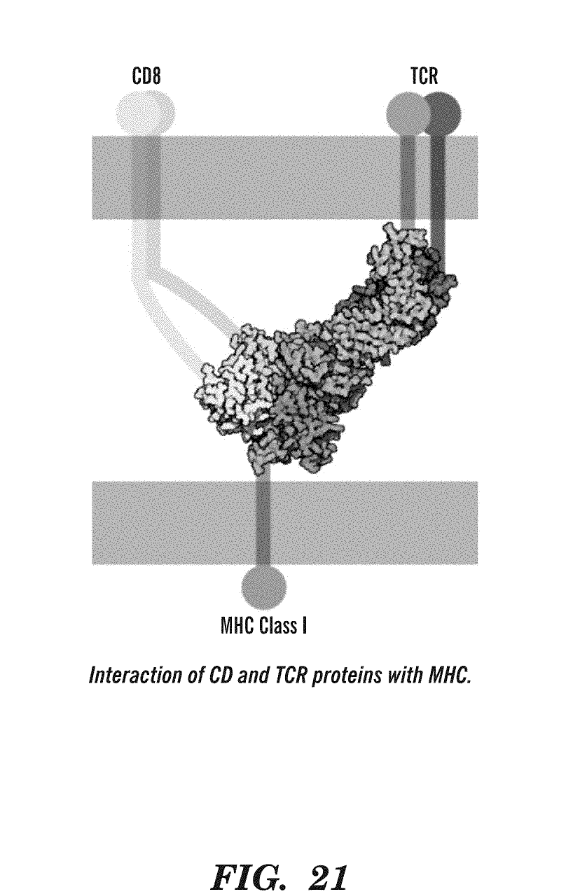

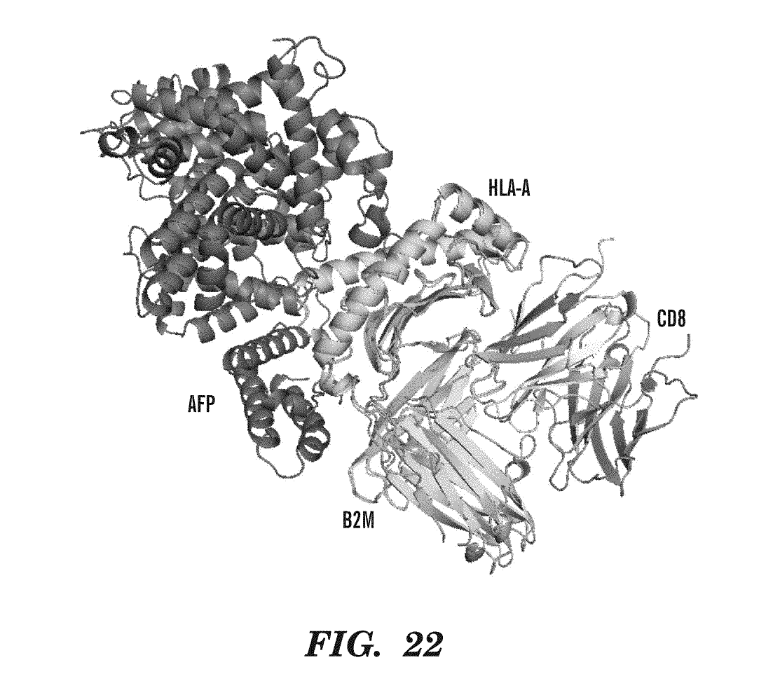

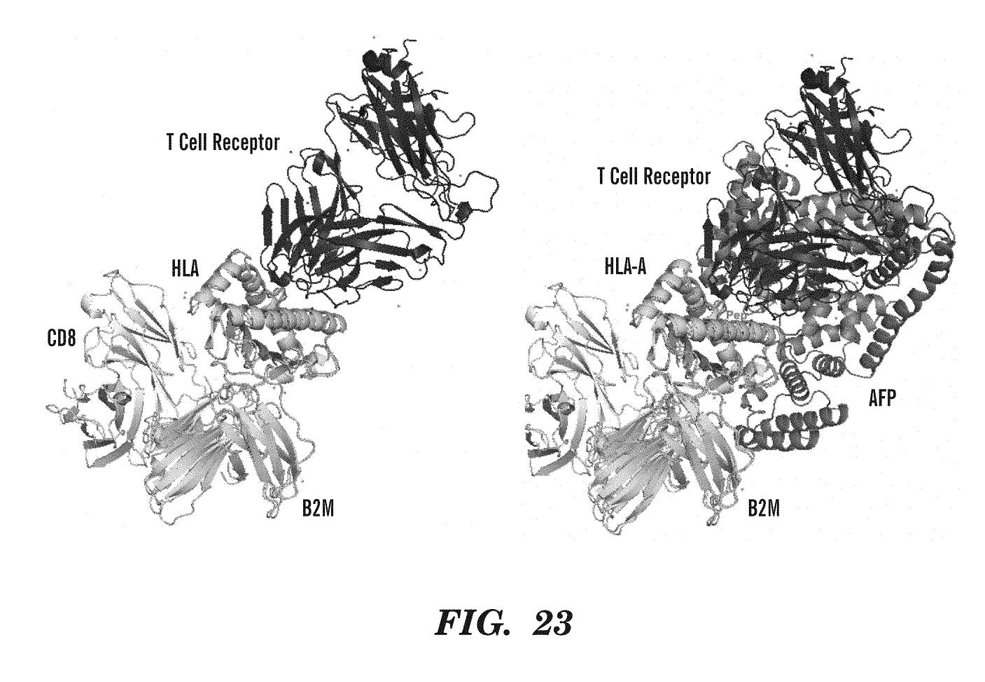

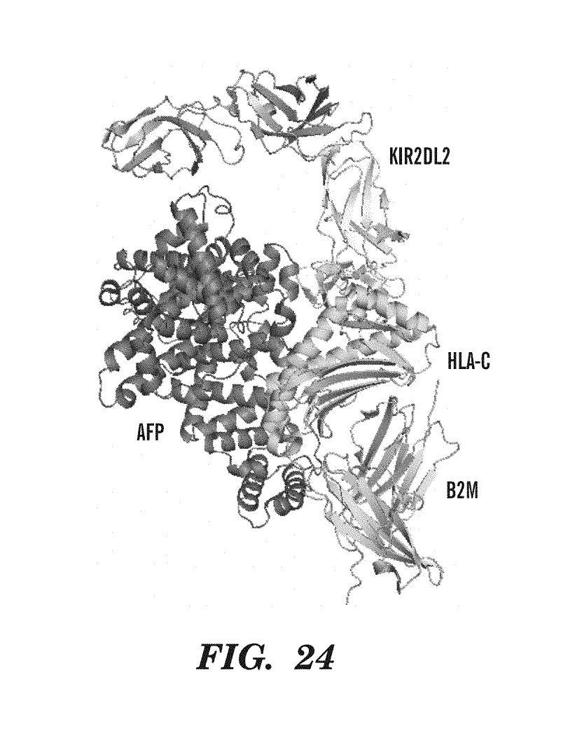

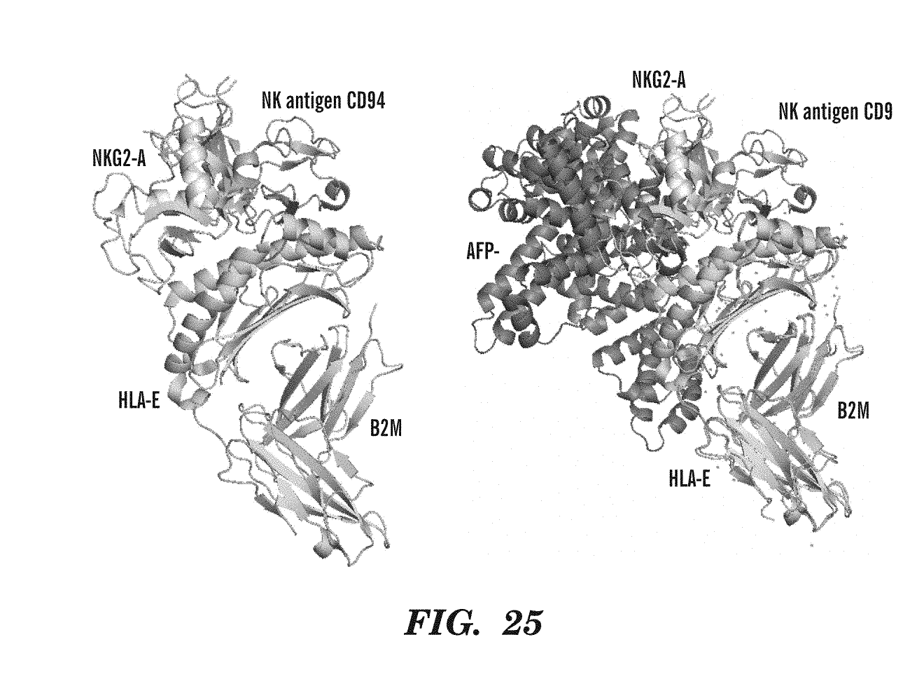

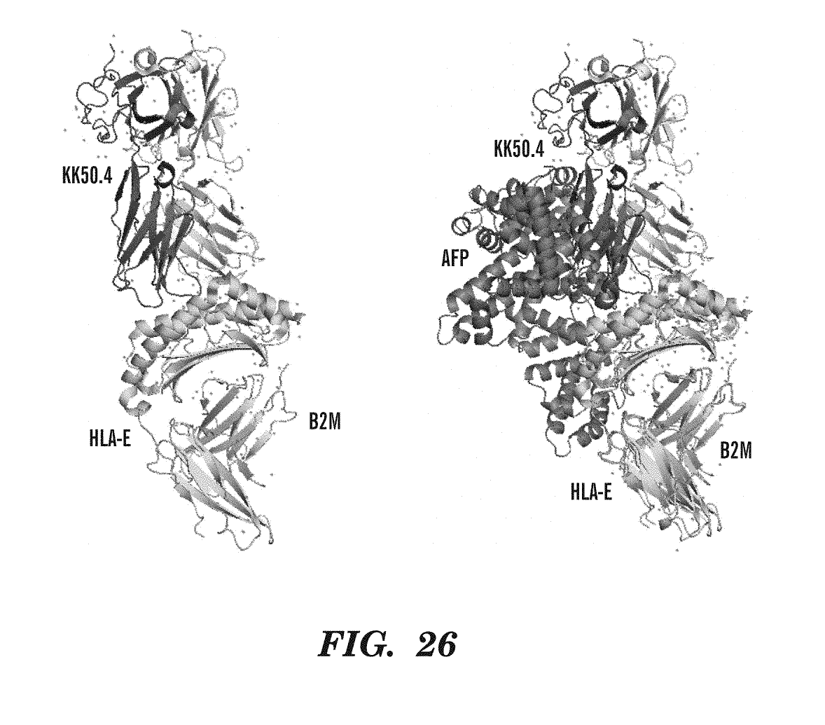

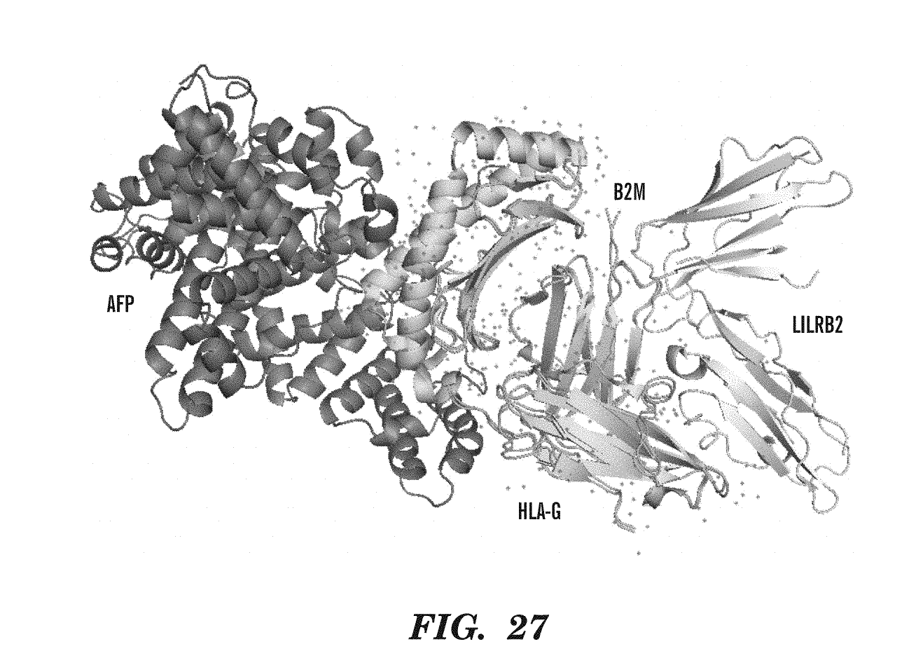

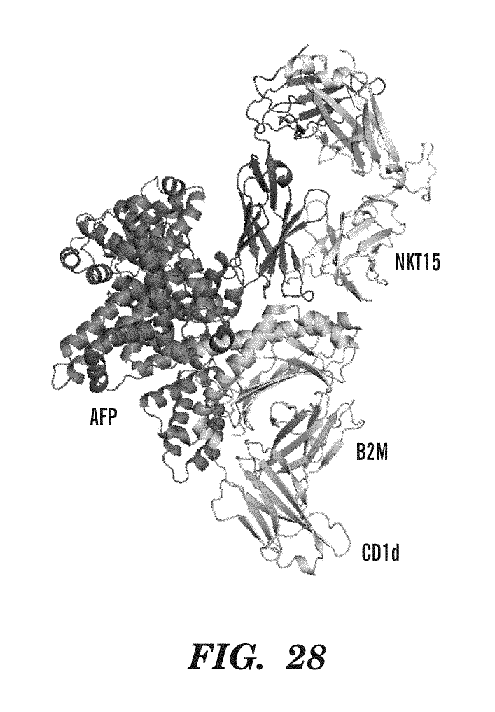

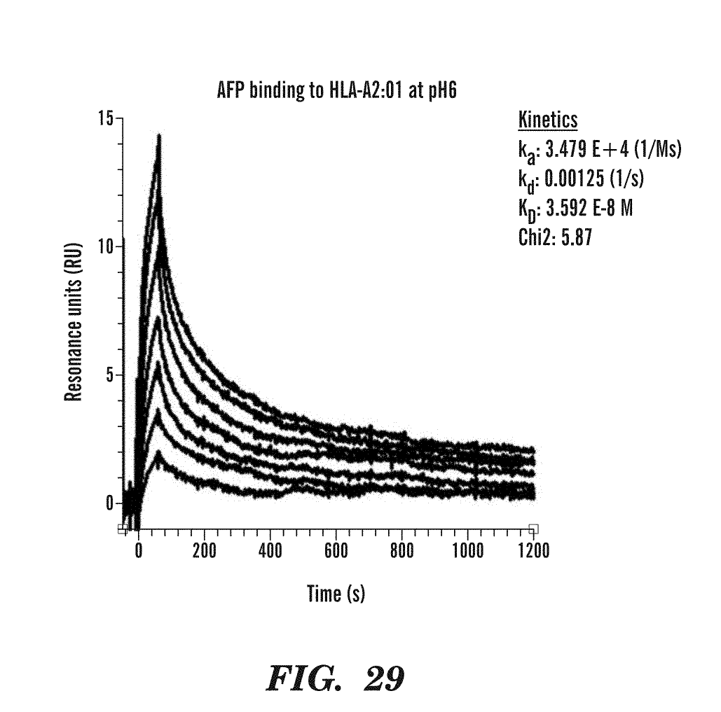

### DETAILED DESCRIPTION

The discoveries described herein demonstrate that AFP binds β₂-microglobulin (β2M) and MHC Class I-related molecules, using different residues and binding sites. As such, the immunosuppressive activity of AFP can be mediated not only by the previously described AFP-FcRn interactions, but can also be mediated by the interaction of AFP with β2M and by the interaction of AFP MHC class I-related molecules, such as HLA-A. As described further herein below, an amino acid alignment established a “phylogenetic tree” or cladogram, which scored the similarity to FcRn and MR1 and provides a hierarchy of the likely strength of interactions of MHC class I-related proteins with AFP, such that FcRn≥HFE≥HLA-A≥HLA-G≥HLA-E≥HLA-B≥MR1≥CD1D≥HLA-C≥ZA2G≥CD1A≥CD1B. Where AFP can interact with any or all of these proteins, any or all of them may be involved in the immunosuppressive effects of AFP, and targeting such interactions can inhibit such immunosuppressive effects or be used to enhance or potentiate AFP immunosuppressive activities.

The compositions and methods described herein also allow for more targeted approaches of manipulating AFP interactions with different binding partners. For example, blocking the β2M interaction sites or interfaces on AFP can allow for blocking AFP interactions with all MHC class I-related molecules, by destabilizing the interaction of β2M with MHC Class I related molecules. Blocking the specific interaction sites on AFP involved in MHC class I-related heavy chain interactions allows for blocking either all MHC class I-related interactions when targeting shared sites on the AFP molecule, or potentially distinct MHC class I related interactions unique to each MHC class I-related heavy chain (e.g., MR1). Accordingly, in some embodiments of the aspects described herein, bispecific (or multispecific) reagents can be used that simultaneously inhibit β2M and heavy chain docking sites or interfaces on AFP. Similarly, AFP peptides, peptidomimetics or small molecules can be used, in some embodiments of the aspects described herein, to block each respective docking site or interface.

Accordingly, compositions and methods are provided herein that relate to the discoveries that alpha fetal protein (AFP) interacts with β2M, and also interacts with MHC Class I-related molecules independent of the β2M interfaces.

### Alpha Fetoprotein (AFP) and MHC Class I-Related Molecules

Alpha-fetoprotein (AFP) is a major plasma protein in the fetus, where it is produced by the yolk sac and liver (Ingram et al., 1981). In an adult, its concentration is very low, except when a tumor, such as a hepatoma or teratoma is present. The alpha-fetoprotein and albumin genes are syntenic, and mammalian AFP and serum albumin genes are believed to have arisen through duplication of an ancestral gene 300 to 500 million years ago. After birth, AFP is down-regulated thousands of fold, such that it is not expressed at high levels in a host under homeostatic conditions. It can become subsequently elevated and expressed at high levels during processes associated with particular types of pathology, such as cancers, particularly in tumors of liver origin (e.g., hepatoma), tumors of the biliary system (e.g., cholangiocarcinoma), and in tumors of primitive origin and that are poorly differentiated, such as teratocarcinomas. In addition, elevated AFP levels can occur during chronic liver inflammatory processes, liver regeneration, and during immune activation, such as allogeneic responses.

Accordingly, the term “AFP” as used herein, refers to the 609 amino acid polypeptide having the amino acid sequence of:

(SEQ ID NO: 1)
MKWVESIFLIFLLNFTESRTLHRNEYGIASILDSYQCTAEISLADLATIF
FAQFVQEATYKEVSKMVKDALTAIEKPTGDEQSSGCLENQLPAFLEELCH
EKEILEKYGHSDCCSQSEEGRHNCFLAHKKPTPASIPLFQVPEPVTSCEA
YEEDRETFMNKFIYEIARRHPFLYAPTILLWAARYDKIIPSCCKAENAVE
CFQTKAATVTKELRESSLLNQHACAVMKNFGTRTFQAITVTKLSQKFTKV
NFTEIQKLVLDVAHVHEHCCRGDVLDCLQDGEKIMSYICSQQDTLSNKIT
ECCKLTTLERGQCIIHAENDEKPEGLSPNLNRFLGDRDFNQFSSGEKNIF
LASFVHEYSRRHPQLAVSVILRVAKGYQELLEKCFQTENPLECQDKGEEE
LQKYIQESQALAKRSCGLFQKLGEYYLQNAFLVAYTKKAPQLTSSELMAI
TRKMAATAATCCQLSEDKLLACGEGAADIIIGHLCIRHEMTPVNPGVGQC
CTSSYANRRPCFSSLVVDETYVPPAFSDDKFIFHKDLCQAQGVALQTMKQ
EFLINLVKQKPQITEEQLEAVIADFSGLLEKCCQGQEQEVCFAEEGQKLI
SKTRAALGV,

as described by, e.g., NP_001125.1, together with any naturally occurring allelic, splice variants, and processed forms thereof. The 18 amino acid signal peptide sequence of AFP is underlined for reference. Typically, AFP refers to human AFP. The sequence of AFP, without the 18 amino acid signal peptide sequence, as used in reference to the interfaces and interacting residues described herein, is provided herein as:

(SEQ ID NO: 2)
RTLHRNEYGIASILDSYQCTAEISLADLATIFFAQFVQEATY
KEVSKMVKDALTAIEKPTGDEQSSGCLENQLPAFLEELCHEKEILEKYGH
SDCCSQSEEGRHNCFLAHKKPTPASIPLFQVPEPVTSCEAYEEDRETFMN
KFIYEIARRHPFLYAPTILLWAARYDKIIPSCCKAENAVECFQTKAATVT
KELRESSLLNQHACAVMKNFGTRTFQAITVTKLSQKFTKVNFTEIQKLVL
DVAHVHEHCCRGDVLDCLQDGEKIMSYICSQQDTLSNKITECCKLTTLER
GQCIIHAENDEKPEGLSPNLNRFLGDRDFNQFSSGEKNIFLASFVHEYSR
RHPQLAVSVILRVAKGYQELLEKCFQTENPLECQDKGEEELQKYIQESQA
LAKRSCGLFQKLGEYYLQNAFLVAYTKKAPQLTSSELMAITRKMAATAAT
CCQLSEDKLLACGEGAADIIIGHLCIRHEMTPVNPGVGQCCTSSYANRRP
CFSSLVVDETYVPPAFSDDKFIFHKDLCQAQGVALQTMKQEFLINLVKQK
PQITEEQLEAVIADFSGLLEKCCQGQEQEVCFAEEGQKLISKTRAALGV

The term “AFP” can also, in some embodiments, be used to refer to truncated forms or fragments of the AFP polypeptide that retain an AFP function or activity of interest as described herein, such as, for example, binding to MHC Class I-related molecules. Reference to any such forms of AFP can be identified in the application, e.g., by “AFP (211-402) of SEQ ID NO: 2.” Specific residues of AFP can be referred to as, for example, “AFP(531) or “F531 of AFP.”

Proteins containing the major histocompatibility complex (MHC) fold form a diverse family of molecules encoded by genes spread throughout the genome. The more recently evolved, classical MHC molecules are represented by highly polymorphic class I and class II types, serve to present peptides of varying size to αβ T cells and are an integral part of vertebrate adaptive immunity. “Classical Class I molecules” comprise an MHC fold derived from a single polypeptide chain (heavy chain) that associates with the nonpolymorphic β2M subunit to form a stable, cell-surface protein. Non-polymorphic Class I-related family members, typically referred to as “non-classical MHC Class I” molecules, for those encoded by genes located within the MHC region (classical MHC class I) and “MHC Class I-like” molecules for those outside the MHC, typically have functions other than peptide presentation—these functions can be immune or non-immune related. As used herein, the term “Class I-related molecule” refers to any classical MHC Class I molecule, non-classical MHC Class I molecule, or MHC Class I-like molecule that associates with β2M, and thus includes, but is not limited to, HLA-A, HLA-B, HLA-C, HLA-E, HLA-F, HLA-G, ZA2G (or ZAG), CD1A, CD1B, CD1C, CD1D, CD1E, HFE, and MR1.

β2-microglobulin or β2M is a non-glycosylated polypeptide composed of 119 amino acids, which includes a nine amino acid leader sequence. Its best characterized function is to interact with and stabilize the tertiary structure of the α-chain of MHC Class I-related molecules. Because it is non-covalently associated with the α-chains, it can be exchanged with the circulating form of β2M, which is present at low levels in serum, urine, and other body fluids under physiological conditions. β2M comprising MHC class I-related molecules are found on almost all normal nucleated cells and on most tumor cells, although the levels of expression may differ among different cells. While some solid tumors express a low density of β2M/MHC class I molecules on their surface to escape host immune surveillance, overexpression of β2M/MHC class I molecules has also been reported on other tumors, including hematological malignancies. As used herein, the terms “β2-microglobulin,” “β2M,” and “β2m” refer to the 119 amino acid polypeptide having the amino acid sequence of:

(SEQ ID NO: 3)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM,

as described by, e.g., NP_004039.1, together with any naturally occurring allelic, splice variants, and processed forms thereof. The 20 amino acid signal peptide sequence of β2M is underlined for reference. The sequence of human β2M, without the 20 amino acid signal peptide sequence, as used in reference to the interfaces and interacting residues described herein, is provided herein as:

(SEQ ID NO: 4)
IQRTPKIQVYSRHPAENGKSNFLNCYVSGFHPSDIEVDLLKNGERIEKVE
HSDLSFSKDWSFYLLYYTEFTPTEKDEYACRVNHVTLSQPKIVKWDRDM.

Typically, β2M refers to human β2M. Reference to interfaces or subsequences of β2M can be identified in the application, e.g., by “β2M (1-9)” or “amino acids 1-9 of SEQ ID NO: 4.” Specific residues of β2M can be referred to as, for example, “β2M(119)”)” or “M119 of β2M.”

Classical MHC class I molecules or Human Leukocyte Antigen (HLA)-A, HLA-B, and HLA-C comprise a 45-kDa α-chain that contains domains α1, α2, and Ig-like domain α3, and an 11.6-kDa light chain called β2-microglobulin (β2M). The α1 and α2 domains of the α-chain are polymorphic. Their polymorphisms frequently occur in three hypervariable regions that form the antigen-binding cleft or peptide-binding region, which is recognized by the T-cell receptor on CD8⁺ T lymphocytes. Domain α3 contains a conserved seven-amino acid loop that binds with CD8 molecules.

As used herein, the terms “Human Leukocyte Antigen-A,” “and “HLA-A” refers to the 365 amino acid mature polypeptide having the amino acid sequence of:

(SEQ ID NO: 5)
MAVMAPRTLLLLLSGALALTQTWAGSHSMRYFFTSVSRPGRGEPRFIAVG
YVDDTQFVRFDSDAASQKMEPRAPWIEQEGPEYWDQETRNMKAHSQTDRA
NLGTLRGYYNQSEDGSHTIQIMYGCDVGPDGRFLRGYRQDAYDGKDYIAL
NEDLRSWTAADMAAQITKRKWEAVHAAEQRRVYLEGRCVDGLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIPIVGIIAGLVLLGAVITGAVVAAVMWRRKSSDRKGGSYTQAASS
DSAQGSDVSLTACKV,

as described by, e.g., NP_001229687.1, together with any naturally occurring allelic, splice variants, and processed forms thereof. The 24 amino acid signal peptide sequence of HLA-A is underlined for reference. The sequence of HLA-A, without the 24 amino acid signal peptide sequence, as used in reference to the interfaces and interacting residues described herein, is provided herein as:

(SEQ ID NO: 6)
GSHSMRYFFTSVSRPGRGEPRFIAVGYVDDTQFVRFDSDAASQKMEPRAP
WIEQEGPEYWDQETRNMKAHSQTDRANLGTLRGYYNQSEDGSHTIQIMYG
CDVGPDGRFLRGYRQDAYDGKDYIALNEDLRSWTAADMAAQITKRKWEAV
HAAEQRRVYLEGRCVDGLRRYLENGKETLQRTDPPKTHMTHHPISDHEAT
LRCWALGFYPAEITLTWQRDGEDQTQDTELVETRPAGDGTFQKWAAVVVP
SGEEQRYTCHVQHEGLPKPLTLRWELSSQPTIPIVGIIAGLVLLGAVITG
AVVAAVMWRRKSSDRKGGSYTQAASSDSAQGSDVSLTACKV.

Reference to interfaces or subsequences of HLA-A can be identified in the application, e.g., by “HLA-A (41-68)” or “amino acids 41-68 of SEQ ID NO: 6.” Specific residues of HLA-A can be referred to as, for example, “HLA-A (41)”)” or “A41 of HLA-A.”

As used herein, the terms “Human Leukocyte Antigen-B,” “and “HLA-B” refer to the 362 amino acid mature polypeptide having the amino acid sequence of:

(SEQ ID NO: 7)
MLVMAPRTVLLLLSAALALTETWAGSHSMRYFYTSVSRPGRGEPRFISVG
YVDDTQFVRFDSDAASPREEPRAPWIEQEGPEYWDRNTQIYKAQAQTDRE
SLRNLRGYYNQSEAGSHTLQSMYGCDVGPDGRLLRGHDQYAYDGKDYIAL
NEDLRSWTAADTAAQITQRKWEAAREAEQRRAYLEGECVEWLRRYLENGK
DKLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVPIVGIVAGLAVLAVVVIGAVVAAVMCRRKSSGGKGGSYSQAACS
DSAQGSDVSLTA,

as described by, e.g., NP_005505.2, together with any naturally occurring allelic, splice variants, and processed forms thereof. The 24 amino acid signal peptide sequence of HLA-B is underlined for reference. The sequence of HLA-B, without the 24 amino acid signal peptide sequence, as used in reference to the interfaces and interacting residues described herein, is provided herein as:

(SEQ ID NO: 8)
GSHSMRYFYTSVSRPGRGEPRFISVGYVDDTQFVRFDSDAASPREEPRA
PWIEQEGPEYWDRNTQIYKAQAQTDRESLRNLRGYYNQSEAGSHTLQSM
YGCDVGPDGRLLRGHDQYAYDGKDYIALNEDLRSWTAADTAAQITQRKW
EAAREAEQRRAYLEGECVEWLRRYLENGKDKLERADPPKTHVTHHPISD
HEATLRCWALGFYPAEITLTWQRDGEDQTQDTELVETRPAGDRTFQKWA
AVVVPSGEEQRYTCHVQHEGLPKPLTLRWEPSSQSTVPIVGIVAGLAVL
AVVVIGAVVAAVMCRRKSSGGKGGSYSQAACSDSAQGSDVSLTA.

Reference to interfaces or subsequences of HLA-B can be identified in the application, e.g., by “HLA-B (41-68)” or “amino acids 41-68 of SEQ ID NO: 8.” Specific residues of HLA-B can be referred to as, for example, “HLA-B (41)”)” or “A41 of HLA-B.”

As used herein, the terms “Human Leukocyte Antigen-C,” “and “HLA-C” refer to the 366 amino acid mature polypeptide having the amino acid sequence of:

(SEQ ID NO: 9)
MRVMAPRALLLLLSGGLALTETWACSHSMRYFDTAVSRPGRGEPRFISVG
YVDDTQFVRFDSDAASPRGEPRAPWVEQEGPEYWDRETQKYKRQAQADRV
SLRNLRGYYNQSEDGSHTLQRMSGCDLGPDGRLLRGYDQSAYDGKDYIAL
NEDLRSWTAADTAAQITQRKLEAARAAEQLRAYLEGTCVEWLRRYLENGK
ETLQRAEPPKTHVTHHPLSDHEATLRCWALGFYPAEITLTWQRDGEDQTQ
DTELVETRPAGDGTFQKWAAVVVPSGQEQRYTCHMQHEGLQEPLTLSWEP
SSQPTIPIMGIVAGLAVLVVLAVLGAVVTAMMCRRKSSGGKGGSCSQAAC
SNSAQGSDESLITCKA,

as described by, e.g., NP_002108.4, together with any naturally occurring allelic, splice variants, and processed forms thereof. The 24 amino acid signal peptide sequence of HLA-C is underlined for reference. The sequence of HLA-C, without the 24 amino acid signal peptide sequence, as used in reference to the interfaces and interacting residues described herein, is provided herein as:

(SEQ ID NO: 10)
CSHSMRYFDTAVSRPGRGEPRFISVGYVDDTQFVRFDSDAASPRGEPRAP
WVEQEGPEYWDRETQKYKRQAQADRVSLRNLRGYYNQSEDGSHTLQRMSG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPAGDGTFQKWAAVVVP
SGQEQRYTCHMQHEGLQEPLTLSWEPSSQPTIPIMGIVAGLAVLVVLAVL
GAVVTAMMCRRKSSGGKGGSCSQAACSNSAQGSDESLITCKA.

Reference to interfaces or subsequences of HLA-C can be identified in the application, e.g., by “HLA-C(41-68)” or “amino acids 41-68 of SEQ ID NO: 10.” Specific residues of HLA-C can be referred to as, for example, “HLA-C(41)”)” or “A41 of HLA-C.”

The non-classical MHC Class I-related proteins, namely HLA-E, HLA-F, and HLA-G in humans, retain the ability to bind and present peptides, although distinct structural features concentrated in the peptide-binding region and their low polymorphism distinguish these proteins from classical MHC Class I molecules.

HLA-E preferentially presents peptides derived from leader sequences of classical class I molecules. Five conserved hydrophobic pockets in the groove of HLA-E anchor the peptides via residues 2, 3, 6, 7, and 9; an extensive hydrogen-bonding network between the heavy chain and the peptide main chain, as well as conserved charged interactions further stabilize the peptides. In addition to roles in innate immunity, HLA-E is capable of eliciting a specific CD8+αβ T cell response to a variety of pathogens such as Salmonella typhi, Mycobacterium tuberculosis, and cytomegalovirus (CMV).

As used herein, the terms “Human Leukocyte Antigen-E,” “and “HLA-E” refer to the 358 amino acid mature polypeptide having the amino acid sequence of:

(SEQ ID NO: 11)
MVDGTLLLLLSEALALTQTWAGSHSLKYFHTSVSRPGRGEPRFISVGYVD
DTQFVRFDNDAASPRMVPRAPWMEQEGSEYWDRETRSARDTAQIFRVNLR
TLRGYYNQSEAGSHTLQWMHGCELGPDGRFLRGYEQFAYDGKDYLTLNED
LRSWTAVDTAAQISEQKSNDASEAEHQRAYLEDTCVEWLHKYLEKGKETL
LHLEPPKTHVTHHPISDHEATLRCWALGFYPAEITLTWQQDGEGHTQDTE
LVETRPAGDGTFQKWAAVVVPSGEEQRYTCHVQHEGLPEPVTLRWKPASQ
PTIPIVGIIAGLVLLGSVVSGAVVAAVIWRKKSSGGKGGSYSKAEWSDSA
QGSESHSL,

as described by, e.g., NP_005507.3, together with any naturally occurring allelic, splice variants, and processed forms thereof. The 21 amino acid signal peptide sequence of HLA-E is underlined for reference. The sequence of HLA-E, without the 21 amino acid signal peptide sequence, as used in reference to the interfaces and interacting residues described herein, is provided herein as:

(SEQ ID NO: 12)
GSHSLKYFHTSVSRPGRGEPRFISVGYVDDTQFVRFDNDAASPRMVPRAP
WMEQEGSEYWDRETRSARDTAQIFRVNLRTLRGYYNQSEAGSHTLQWMHG
CELGPDGRFLRGYEQFAYDGKDYLTLNEDLRSWTAVDTAAQISEQKSNDA
SEAEHQRAYLEDTCVEWLHKYLEKGKETLLHLEPPKTHVTFIHPISDHEA
TLRCWALGFYPAEITLTWQQDGEGHTQDTELVETRPAGDGTFQKWAAVVV
PSGEEQRYTCHVQHEGLPEPVTLRWKPASQPTIPIVGIIAGLVLLGSVVS
GAVVAAVIWRKKSSGGKGGSYSKAEWSDSAQGSESHSL.

Reference to interfaces or subsequences of HLA-E can be identified in the application, e.g., by “HLA-E (41-68)” or “amino acids 41-68 of SEQ ID NO: 12.” Specific residues of HLA-E can be referred to as, for example, “HLA-E (41)”)” or “A41 of HLA-E.”

As used herein, the terms “Human Leukocyte Antigen-F,” “and “HLA-F” refer to the 346 amino acid mature polypeptide having the amino acid sequence of:

(SEQ ID NO: 13)
MAPRSLLLLLSGALALTDTWAGSHSLRYFSTAVSRPGRGEPRYIAVEYVD
DTQFLRFDSDAAIPRMEPREPWVEQEGPQYWEWTTGYAKANAQTDRVALR
NLLRRYNQSEAGSHTLQGMNGCDMGPDGRLLRGYHQHAYDGKDYISLNED
LRSWTAADTVAQITQRFYEAEEYAEEFRTYLEGECLELLRRYLENGKETL
QRADPPKAHVAHHPISDHEATLRCWALGFYPAEITLTWQRDGEEQTQDTE
LVETRPAGDGTFQKWAAVVVPPGEEQRYTCHVQHEGLPQPLILRWEQSPQ
PTIPIVGIVAGLVVLGAVVTGAVVAAVMWRKKSSDRNRGSYSQAAV,

as described by, e.g., NP_061823.2, together with any naturally occurring allelic, splice variants, and processed forms thereof. The 21 amino acid signal peptide sequence of HLA-F is underlined for reference. The sequence of HLA-F, without the 21 amino acid signal peptide sequence, as used in reference to the interfaces and interacting residues described herein, is provided herein as:

(SEQ ID NO: 14)
GSHSLRYFSTAVSRPGRGEPRYIAVEYVDDTQFLRFDSDAAIPRMEPREP
WVEQEGPQYWEWTTGYAKANAQTDRVALRNLLRRYNQSEAGSHTLQGMNG
CDMGPDGRLLRGYHQHAYDGKDYISLNEDLRSWTAADTVAQITQRFYEAE
EYAEEFRTYLEGECLELLRRYLENGKETLQRADPPKAHVAHHPISDHEAT
LRCWALGFYPAEITLTWQRDGEEQTQDTELVETRPAGDGTFQKWAAVVVP
PGEEQRYTCHVQHEGLPQPLILRWEQSPQPTIPIVGIVAGLVVLGAVVTG
AVVAAVMWRKKSSDRNRGSYSQAAV.

Reference to interfaces or subsequences of HLA-F can be identified in the application, e.g., by “HLA-F (41-68)” or “amino acids 41-68 of SEQ ID NO: 14.” Specific residues of HLA-F can be referred to as, for example, “HLA-F(41)”)” or “A41 of HLA-E.”

HLA-G is similar in overall structure to HLA-E and is expressed in various forms (membrane-bound and soluble isoforms) as a result of alternative mRNA splicing. HLA-G is also a ligand for inhibitory receptors expressed on NK cells. HLA-G is predominantly expressed on fetal extravillous trophoblasts, providing signals to NK cells, macrophages, and monocytes through leukocyte immunoglobulin-like receptor-1 (LILRB1, LIR-1, or ILT2), LILRB2 (LIR2 or ILT-4) (26), and killer immunoglobulin-like receptor 2DL4 (KIR2DL4) (27, 28) that help to maintain maternal tolerance and promote fetal development. HLA-G has also been implicated in tumor surveillance via peptide presentation to CD8+ T cells. HLA-G peptides have preferences at three positions: Pro or small hydrophobic residue at position 3, Pro or Gly at position 4, and Leu at the C terminus (P9). HLA-G can exist as a disulfide-linked dimer in solution and on the cell surface.

As used herein, the terms “Human Leukocyte Antigen-G,” “and “HLA-G” refer to the 338 amino acid polypeptide having the amino acid sequence of:

(SEQ ID NO: 15)
MVVMAPRTLFLLLSGALTLTETWAGSHSMRYFSAAVSRPGRGEPRFIAMG
YVDDTQFVRFDSD SACPRMEPRAPWVEQEGPEYWEEETRNTKAHAQTDR
MNLQTLRGYYNQSEASSHTLQWMIGCDLGSDGRLLRGYEQYAYDGKDYLA
LNEDLRSWTAADTAAQISKRKCEAANVAEQRRAYLEGTCVEWLHRYLENG
KEMLQRADPPKTHVTHHPVFDYEATLRCWALGFYPAEIILTWQRDGEDQT
QDVELVETRPAGDGTFQKWAAVVVPSGEEQRYTCHVQHEGLPEPLMLRWK
QSSLPTIPIMGIVAGLVVLAAVVTGAAVAAVLWRKKSSD,

as described by, e.g., NP_002118.1, together with any naturally occurring allelic, splice variants, and processed forms thereof. The 24 amino acid signal peptide sequence of HLA-G is underlined for reference. The sequence of HLA-G, without the 24 amino acid signal peptide sequence, as used in reference to the interfaces and interacting residues described herein, is provided herein as:

(SEQ ID NO: 16)
GSHSMRYFSAAVSRPGRGEPRFIAMGYVDDTQFVRFDSDSACPRMEPRAP
WVEQEGPEYWEEETRNTKAHAQTDRMNLQTLRGYYNQSEASSHTLQWMIG
CDLGSDGRLLRGYEQYAYDGKDYLALNEDLRSWTAADTAAQISKRKCEAA
NVAEQRRAYLEGTCVEWLHRYLENGKEMLQRADPPKTHVTFIHPVFDYEA
TLRCWALGFYPAEIILTWQRDGEDQTQDVELVETRPAGDGTFQKWAAVVV
PSGEEQRYTCHVQHEGLPEPLMLRWKQSSLPTIPIMGIVAGLVVLAAVVT
GAAVAAVLWRKKSSD.

Reference to interfaces or subsequences of HLA-G can be identified in the application, e.g., by “HLA-G (41-68)” or “amino acids 41-68 of SEQ ID NO: 16.” Specific residues of HLA-G can be referred to as, for example, “HLA-G (41)”)” or “A41 of HLA-G.”

ZA2G or ZAG (zinc-α2-glycoprotein) is an example of an MHC scaffold modified specifically for nonimmune functions. It is expressed as a soluble heavy chain independent of β2M and is found at high concentrations in serum and other bodily fluids. ZA2G stimulates lipid breakdown in adipocytes, contributes to the reduction of fat stores in animal models, and has been linked to the fat-depletion effect of cachexia, a wasting phenomenon present in many patients with cancer, AIDS, and other life-threatening diseases. The structure of ZA2G is highly similar in backbone conformation to classical class I molecules, despite sharing only 30-40% amino acid identity, with modifications to the putative antigen-binding groove that would likely prevent peptide presentation. Instead, ZAG's groove is adapted to binding small, hydrophobic molecules similar in structure to polyethylene glycols (PEGs) or fatty acids. Mutagenesis of the binding groove revealed an important role for R73, which is the only charged residue located in the hydrophobic binding groove lined with aromatic residues. This charged residue extends into the groove from the α1-helix and stabilizes the groove's open state, as mutation to alanine abrogates binding of ligand and closes the groove to solvent. There is evidence that ZA2G binds directly to the β-adrenoreceptors 2 (β2-AR) and 3 (β3-AR), but not β1-AR.

As used herein, the terms “zinc-α2-glycoprotein,” “ZA2G,” and “ZAG” refer to the XXX amino acid polypeptide having the amino acid sequence of:

(SEQ ID NO: 17)
MVRMVPVLLSLLLLLGPAVPQENQDGRYSLTYIYTGLSKHVEDVPAFQAL
GSLNDLQFFRYNSKDRKSQPMGLWRQVEGMEDWKQDSQLQKAREDIFMET
LKDIVEYYNDSNGSHVLQGRFGCEIENNRSSGAFWKYYYDGKDYIEFNKE
IPAWVPFDPAAQITKQKWEAEPVYVQRAKAYLEEECPATLRKYLKYSKNI
LDRQDPPSVVVTSHQAPGEKKKLKCLAYDFYPGKIDVHWTRAGEVQEPEL
RGDVLHNGNGTYQSWVVVAVPPQDTAPYSCHVQHSSLAQPLVVPWEAS,

as described by, e.g., NP_001176.1, together with any naturally occurring allelic, splice variants, and processed forms thereof. The 21 amino acid signal peptide sequence of ZA2G is underlined for reference. The sequence of ZA2G, without the 21 amino acid signal peptide sequence, as used in reference to the interfaces and interacting residues described herein, is provided herein as:

(SEQ ID NO: 18)
QENQDGRYSLTYIYTGLSKHVEDVPAFQALGSLNDLQFFRYNSKDRKSQP
MGLWRQVEGMEDWKQDSQLQKAREDIFMETLKDIVEYYNDSNGSHVLQGR
FGCEIENNRSSGAFWKYYYDGKDYIEFNKEIPAWVPFDPAAQITKQKWEA
EPVYVQRAKAYLEEECPATLRKYLKYSKNILDRQDPPSVVVTSHQAPGEK
KKLKCLAYDFYPGKIDVHWTRAGEVQEPELRGDVLHNGNGTYQSWVVVAV
PPQDTAPYSCHVQHSSLAQPLVVPWEAS.

Reference to interfaces or subsequences of ZA2G can be identified in the application, e.g., by “ZA2G (45-72)” or “amino acids 45-72 of SEQ ID NO: 18” Specific residues of ZA2G can be referred to as, for example, “ZA2G(45)”)” or “D45 of ZA2G.”

The human hemochromatosis protein, or HFE, was discovered owing to its genetic link with an iron storage disorder called hereditary hemochromatosis. Common mutations in HFE, for example Cys260Tyr (which disrupts HFE's association with β2M), result in excessive iron deposition in tissues and organs, and this is thought to be due to HFE's ability to regulate iron uptake by transferrin (TO by associating with the Tf receptor (TfR). Unlike other ligand-presenting class I-like molecules, HFE's binding groove is closed due to a 4-A° translation of the α1-helix closer to the α2-helix and substitutions of larger side chains at positions lining the groove. HFE has not been shown to present or associate with ligands (including iron), although its association with the TfR has been well studied biophysically and structurally. TfR binds HFE on the platform domain, with most of the contacts centered on the α1-helix. Like Tf's association with TfR, HFE associates with TfR with a pH dependency.

As used herein, the terms “human hemochromatosis protein,” and “HFE” refer to the 348 amino acid polypeptide having the amino acid sequence of:

(SEQ ID NO: 19)
MGPRARPALLLLMLLQTAVLQGRLLRSHSLHYLFMGASEQDLGLSLFEAL
GYVDDQLFVFYDHESRRVEPRTPWVSSRISSQMWLQLSQSLKGWDHMFTV
DFWTIMENHNHSKESHTLQVILGCEMQEDNSTEGYWKYGYDGQDHLEFCP
DTLDWRAAEPRAWPTKLEWERHKIRARQNRAYLERDCPAQLQQLLELGRG
VLDQQVPPLVKVTHHVTSSVTTLRCRALNYYPQNITMKWLKDKQPMDAKE
FEPKDVLPNGDGTYQGWITLAVPPGEEQRYTCQVEHPGLDQPLIVIWEPS
PSGTLVIGVISGIAVFVVILFIGILFIILRKRQGSRGAMGHYVLAERE,

as described by, e.g., NP_000401.1, together with any naturally occurring allelic, splice variants, and processed forms thereof. The 22 amino acid signal peptide sequence of HFE is underlined for reference. The sequence of ZA2G, without the 21 amino acid signal peptide sequence, as used in reference to the interfaces and interacting residues described herein, is provided herein as:

(SEQ ID NO: 20)
RLLRSHSLHYLFMGASEQDLGLSLFEALGYVDDQLFVFYDHESRRVEPR
TPWVSSRISSQMWLQLSQSLKGWDHMFTVDFWTIMENHNHSKESHTLQV
ILGCEMQEDNSTEGYWKYGYDGQDHLEFCPDTLDWRAAEPRAWPTKLEW
ERHKIRARQNRAYLERDCPAQLQQLLELGRGVLDQQVPPLVKVTHHVTS
SVTTLRCRALNYYPQNITMKWLKDKQPMDAKEFEPKDVLPNGDGTYQGW
ITLAVPPGEEQRYTCQVEHPGLDQPLIVIWEPSPSGTLVIGVISGIAVF
VVILFIGILFIILRKRQGSRGAMGHYVLAERE.

Reference to interfaces or subsequences of HFE can be identified in the application, e.g., by “HFE (45-72)” or “amino acids 45-72 of SEQ ID NO: 20.” Specific residues of HFE can be referred to as, for example, “HFE(45)” or “D45 of HFE.”

As used herein, the term “MR1” refers to the 341 amino acid polypeptide having the amino acid sequence of:

(SEQ ID NO: 21)
MGELMAFLLPLIIVLMVKHSDSRTHSLRYFRLGVSDPIHGVPEFISVGYV
DSHPITTYDSVTRQKEPRAPWMAENLAPDHWERYTQLLRGWQQMFKVELK
RLQRHYNHSGSHTYQRMIGCELLEDGSTTGFLQYAYDGQDFLIFNKDTLS
WLAVDNVAHTIKQAWEANQHELLYQKNWLEEECIAWLKRFLEYGKDTLQR
TEPPLVRVNRKETFPGVTALFCKAHGFYPPEIYMTWMKNGEEIVQEIDYG
DILPSGDGTYQAWASIELDPQSSNLYSCHVEHCGVHMVLQVPQESETIPL
VMKAVSGSIVLVIVLAGVGVLVWRRRPREQNGAIYLPTPDR,

as described by, e.g., NP_001522.1, together with any naturally occurring allelic, splice variants, and processed forms thereof. The 22 amino acid signal peptide sequence of MR1 is underlined for reference. The sequence of MR1, without the 22 amino acid signal peptide sequence, as used in reference to the interfaces and interacting residues described herein, is provided herein as:

(SEQ ID NO: 22)
RTHSLRYFRLGVSDPIHGVPEFISVGYVDSHPITTYDSVTRQKEPRAPWM
AENLAPDHWERYTQLLRGWQQMFKVELKRLQRHYNHSGSHTYQRMIGCEL
LEDGSTTGFLQYAYDGQDFLIFNKDTLSWLAVDNVAHTIKQAWEANQHEL
LYQKNWLEEECIAWLKRFLEYGKDTLQRTEPPLVRVNRKETFPGVTALFC
KAHGFYPPEIYMTWMKNGEEIVQEIDYGDILPSGDGTYQAWASIELDPQS
SNLYSCHVEHCGVHMVLQVPQESETIPLVMKAVSGSIVLVIVLAGVGVLV
WRRRPREQNGAIYLPTPDR.

Reference to interfaces or subsequences of MR1 can be identified in the application, e.g., by “MR1(40-67)” or “amino acids 40-67 of SEQ ID NO: 22.” Specific residues of MR1 can be referred to as, for example, “MR1(40)” or “T40 of MR1.”

The human CD1 family consists of two groups: group 1 (CD1A, CD1B, CD1C and CD1E) and group 2(CD1D). The CD1 group of proteins is MHC class I-related and associates with β2M. The CD1 family members are assembled in the endoplasmic reticulum where they acquire lipid antigen for presentation on the cell surface to either type 1 natural killer T (NKT) cells that possess an invariant T cell receptor alpha chain or type 2 NKT cells that express a semi-invariant group of T cell receptor alpha chains. Once on the cell surface the CD1 proteins can internalize and acquire different lipid antigenic specificities for presentation to NKT cells upon recycling to the cell surface. As demonstrated herein, alpha-fetoprotein (AFP) interacts with and binds to all CD1 family heavy chains and their associated β2M molecule with a hierarchy of CD1D>CD1A>CD1B and also CD1C and CD1E based upon sequence homology with CD1A and CD1B.

As used herein, the term “CD1A” refers to the 327 amino acid polypeptide having the amino acid sequence of:

(SEQ ID NO: 23)
MLFLLLPLLAVLPGDGNADGLKEPLSFHVTWIASFYNHSWKQNLVSGWLS
DLQTHTWDSNSSTIVFLCPWSRGNFSNEEWKELETLFRIRTIRSFEGIRR
YAHELQFEYPFEIQVTGGCELHSGKVSGSFLQLAYQGSDFVSFQNNSWLP
YPVAGNMAKHFCKVLNQNQHENDITHNLLSDTCPRFILGLLDAGKAHLQR
QVKPEAWLSHGPSPGPGHLQLVCHVSGFYPKPVWVMWMRGEQEQQGTQRG
DILPSADGTWYLRATLEVAAGEAADLSCRVKHSSLEGQDIVLYWEHHSSV
GFIILAVIVPLLLLIGLALWFRKRCFC,

as described by, e.g., NP_001754.2, together with any naturally occurring allelic, splice variants, and processed forms thereof. The 18 amino acid signal peptide sequence of CD1A is underlined for reference. The sequence of CD1A, without the 18 amino acid signal peptide sequence, as used in reference to the interfaces and interacting residues described herein, is provided herein as:

(SEQ ID NO: 24)
DGLKEPLSFHVTWIASFYNHSWKQNLVSGWLSDLQTHTWDSNSSTIVFLC
PWSRGNFSNEEWKELETLFRIRTIRSFEGIRRYAHELQFEYPFEIQVTGG
CELHSGKVSGSFLQLAYQGSDFVSFQNNSWLPYPVAGNMAKHFCKVLNQN
QHENDITHNLLSDTCPRFILGLLDAGKAHLQRQVKPEAWLSHGPSPGPGH
LQLVCHVSGFYPKPVWVMWMRGEQEQQGTQRGDILPSADGTWYLRATLEV
AAGEAADLSCRVKHSSLEGQDIVLYWEHHSSVGFIILAVIVPLLLLIGLA
LWFRKRCFC.

Reference to interfaces or subsequences of CD1A can be identified in the application, e.g., by “CD1A(41-71)” or “amino acids 41-71 of SEQ ID NO: 24” Specific residues of CD1A can be referred to as, for example, “CD1A(41)” or “D41 of CD1A.”

As used herein, the term “CD1B” refers to the 333 amino acid polypeptide having the amino acid sequence of:

(SEQ ID NO: 25)
MLLLPFQLLAVLFPGGNSEHAFQGPTSFHVIQTSSFTNSTWAQTQGSGWL
DDLQIHGWDSDSGTAIFLKPWSKGNFSDKEVAELEEIFRVYIFGFAREVQ
DFAGDFQMKYPFEIQGIAGCELHSGGAIVSFLRGALGGLDFLSVKNASCV
PSPEGGSRAQKFCALIIQYQGIMETVRILLYETCPRYLLGVLNAGKADLQ
RQVKPEAWLSSGPSPGPGRLQLVCHVSGFYPKPVWVMWMRGEQEQQGTQL
GDILPNANWTWYLRATLDVADGEAAGLSCRVKHSSLEGQDIILYWRNPTS
IGSIVLAIIVPSLLLLLCLALWYMRRRSYQNIP,

as described by, e.g., NP_001755.1, together with any naturally occurring allelic, splice variants, and processed forms thereof. The 17 amino acid signal peptide sequence of CD1B is underlined for reference. The sequence of CD1B, without the 17 amino acid signal peptide sequence, as used in reference to the interfaces and interacting residues described herein, is provided herein as:

(SEQ ID NO: 26)
SEHAFQGPTSFHVIQTSSFTNSTWAQTQGSGWLDDLQIHGWDSDSGTAIF
LKPWSKGNFSDKEVAELEEIFRVYIFGFAREVQDFAGDFQMKYPFEIQGI
AGCELHSGGAIVSFLRGALGGLDFLSVKNASCVPSPEGGSRAQKFCALII
QYQGIMETVRILLYETCPRYLLGVLNAGKADLQRQVKPEAWLSSGPSPGP
GRLQLVCHVSGFYPKPVWVMWMRGEQEQQGTQLGDILPNANWTWYLRATL
DVADGEAAGLSCRVKHSSLEGQDIILYWRNPTSIGSIVLAIIVPSLLLLL
CLALWYMRRRSYQNIP.

Reference to interfaces or subsequences of CD1B can be identified in the application, e.g., by “CD1B(41-71)” or “amino acids 41-71 of SEQ ID NO: 26.” Specific residues of CD1B can be referred to as, for example, “CD1B(41)” or “D41 of CD1B.”

As used herein, the term “CD1C” refers to the 333 amino acid polypeptide having the amino acid sequence of:

(SEQ ID NO: 27)
MLFLQFLLLALLLPGGDNADASQEHVSFHVIQIFSFVNQSWARGQGSGWL
DELQTHGWDSESGTIIFLFMWSKGNFSNEELSDLELLFRFYLFGLTREIQ
DHASQDYSKYPFEVQVKAGCELHSGKSPEGFFQVAFNGLDLLSFQNTTWV
PSPGCGSLAQSVCHLLNHQYEGVTETVYNLIRSTCPRFLLGLLDAGKMYV
HRQVRPEAWLSSRPSLGSGQLLLVCHASGFYPKPVWVTWMRNEQEQLGTK
HGDILPNADGTWYLQVILEVASEEPAGLSCRVRHSSLGGQDIILYWGHHF
SMNWIALVVIVPLVILIVLVLWFKKHCSYQDIL,

as described by, e.g., NP_001756.2, together with any naturally occurring allelic, splice variants, and processed forms thereof. The 17 amino acid signal peptide sequence of CD is underlined for reference. The sequence of CD1C, without the 17 amino acid signal peptide sequence, as used in reference to the interfaces and interacting residues described herein, is provided herein as:

(SEQ ID NO: 28)
NADASQEHVSFHVIQIFSFVNQSWARGQGSGWLDELQTHGWDSESGTIIF
LFMWSKGNFSNEELSDLELLFRFYLFGLTREIQDHASQDYSKYPFEVQVK
AGCELHSGKSPEGFFQVAFNGLDLLSFQNTTWVPSPGCGSLAQSVCHLLN
HQYEGVTETVYNLIRSTCPRFLLGLLDAGKMYVHRQVRPEAWLSSRPSLG
SGQLLLVCHASGFYPKPVWVTWMRNEQEQLGTKHGDILPNADGTWYLQVI
LEVASEEPAGLSCRVRHSSLGGQDIILYWGHHFSMNWIALVVIVPLVILI
VLVLWFKKHCSYQDIL.

Reference to interfaces or subsequences of CD1C can be identified in the application, e.g., by “CD1C(41-71)” or “amino acids 41-71 of SEQ ID NO: 28.” Specific residues of CD1C can be referred to as, for example, “CD1C(41)” or “D41 of CD1C.”

As used herein, the term “CD1D” refers to the 335 amino acid polypeptide having the amino acid sequence of:

(SEQ ID NO: 29)
MGCLLFLLLWALLQAWGSAEVPQRLFPLRCLQISSFANSSWTRTDGLAWL
GELQTHSWSNDSDTVRSLKPWSQGTFSDQQWETLQHIFRVYRSSFTRDVK
EFAKMLRLSYPLELQVSAGCEVHPGNASNNFFHVAFQGKDILSFQGTSWE
PTQEAPLWVNLAIQVLNQDKWTRETVQWLLNGTCPQFVSGLLESGKSELK
KQVKPKAWLSRGPSPGPGRLLLVCHVSGFYPKPVWVKWMRGEQEQQGTQP
GDILPNADETWYLRATLDVVAGEAAGLSCRVKHSSLEGQDIVLYWGGSYT
SMGLIALAVLACLLFLLIVGFTSRFKRQTSYQGVL,

as described by, e.g., NP_001757.1, together with any naturally occurring allelic, splice variants, and processed forms thereof. The 19 amino acid signal peptide sequence of CD1D is underlined for reference. The sequence of CD1D, without the 19 amino acid signal peptide sequence, as used in reference to the interfaces and interacting residues described herein, is provided herein as:

(SEQ ID NO: 30)
EVPQRLFPLRCLQISSFANSSWTRTDGLAWLGELQTHSWSNDSDTVRSLK
PWSQGTFSDQQWETLQHIFRVYRSSFTRDVKEFAKMLRLSYPLELQVSAG
CEVHPGNASNNFFHVAFQGKDILSFQGTSWEPTQEAPLWVNLAIQVLNQD
KWTRETVQWLLNGTCPQFVSGLLESGKSELKKQVKPKAWLSRGPSPGPGR
LLLVCHVSGFYPKPVWVKWMRGEQEQQGTQPGDILPNADETWYLRATLDV
VAGEAAGLSCRVKHSSLEGQDIVLYWGGSYTSMGLIALAVLACLLFLLIV
GFTSRFKRQTSYQGVL.

Reference to interfaces or subsequences of CD1D can be identified in the application, e.g., by “CD1D(45-71)” or “amino acids 45-71 of SEQ ID NO: 30.” Specific residues of CD1D can be referred to as, for example, “CD1D(45)” or “D45 of CD1D.”

As used herein, the term “CD1E” refers to the 388 amino acid polypeptide having the amino acid sequence of:

(SEQ ID NO: 31)
MLLLFLLFEGLCCPGENTAAPQALQSYHLAAEEQLSFRMLQTSSFANHSW
AHSEGSGWLGDLQTHGWDTVLGTIRFLKPWSHGNFSKQELKNLQSLFQLY
FHSFIQIVQASAGQFQLEYPFEIQILAGCRMNAPQIFLNMAYQGSDFLSF
QGISWEPSPGAGIRAQNICKVLNRYLDIKEILQSLLGHTCPRFLAGLMEA
GESELKRKVKPEAWLSCGPSPGPGRLQLVCHVSGFYPKPVWVMWMRGEQE
QRGTQRGDVLPNADETWYLRATLDVAAGEAAGLSCRVKHSSLGGHDLIIH
WGGYSIFLILICLTVIVTLVILVVVDSRLKKQSSNKNILSPHTPSPVFLM
GANTQDTKNSRHQFCLAQVSWIKNRVLKKWKTRLNQLW,

as described by, e.g., NP_112155.2, together with any naturally occurring allelic, splice variants, and processed forms thereof. The 19 amino acid signal peptide sequence of CD1E is underlined for reference. The sequence of CD1E, without the 19 amino acid signal peptide sequence, as used in reference to the interfaces and interacting residues described herein, is provided herein as:

(SEQ ID NO: 32)
APQALQSYHLAAEEQLSFRMLQTSSFANHSWAHSEGSGWLGDLQTHGWDT
VLGTIRFLKPWSHGNFSKQELKNLQSLFQLYFHSFIQIVQASAGQFQLEY
PFEIQILAGCRMNAPQIFLNMAYQGSDFLSFQGISWEPSPGAGIRAQNIC
KVLNRYLDIKEILQSLLGHTCPRFLAGLMEAGESELKRKVKPEAWLSCGP
SPGPGRLQLVCHVSGFYPKPVWVMWMRGEQEQRGTQRGDVLPNADETWYL
RATLDVAAGEAAGLSCRVKHSSLGGHDLIIHWGGYSIFLILICLTVIVTL
VILVVVDSRLKKQSSNKNILSPHTPSPVFLMGANTQDTKNSRHQFCLAQV
SWIKNRVLKKWKTRLNQLW.

Reference to interfaces or subsequences of CD1E can be identified in the application, e.g., by “CD1C(41-71)” or “amino acids 41-71 of SEQ ID NO: 32.” Specific residues of CD1E can be referred to as, for example, “CD1E(41)” or “D41 of CD1E.”

The discoveries described herein that demonstrate that AFP binds β2M and MHC Class I-related molecules provides novel compositions and methods for the treatment of conditions in which modulating the level of AFP is therapeutic. As demonstrated herein, alpha-fetoprotein (AFP) interacts with and binds to MHC Class I-related molecules and β2M. As shown herein, based on high homology between HSA and AFP, a structural model of AFP was built and superimposed on FcRn:HSA:Fc-YTE structure (PDB ID 4N0U) with RMSD of 0.072. This AFP model demonstrates that AFP makes novel interactions with β2M with strong affinity. Modelling studies also indicated novel interactions between AFP and other MHC class-1 molecules, such as MR1. For example, the MR1 crystal structure (PDB ID 4N0U) superimposed on FcRn crystal structure (PDB ID 4N0U) with RMSD of 2.8. Modelling analysis revealed 1278 A² interface area of AFP: MR1 interactions and 6.4 kcal/mol gain in solvation free energy upon binding.

Accordingly, provided herein, in some aspects, are compositions and methods to inhibit or reduce AFP and MHC Class I-related molecule interactions in diseases or disorders associated with AFP-mediated immunosuppression. Also provided herein, in some aspects, are compositions and methods to enhance or potentiate AFP and MHC Class I-related molecule interactions in diseases or disorders associated with decreased AFP levels or diseases or disorders where increasing AFP levels is therapeutic, such as subjects with autoimmune disease or otherwise in need of increasing immunosuppression. Such interactions can be modulated, in some aspects, by targeting the interface(s) between AFP and β2M, thereby modulating the subsequent or consequent docking of and non-covalent interactions between β2M and MHC Class I-related molecules. In other aspects, such interactions can be modulated by directly targeting the interface(s) between AFP and MHC Class I-related molecules.

Provided herein, in some aspects, are compositions, such as pharmaceutical compositions, comprising an inhibitor of AFP-β2M interactions and a pharmaceutically acceptable carrier, wherein the inhibitor of AFP-β2M interactions inhibits binding between alpha-fetoprotein (AFP) and β2M.

As used herein, the terms “inhibitor of AFP-β2M (β2-microglobulin) interactions,” or “AFP-β2M (β2-microglobulin) inhibitor,” refer to a molecule or agent that significantly blocks, inhibits, reduces, or interferes with the interaction between AFP and β2M (β2-microglobulin), and their resultant biological or functional activity in vitro, in situ, and/or in vivo, including docking of MHC Class-I related molecules with β2M, activity of downstream pathways mediated by AFP binding to β2M and signaling. These include, for example: transcytosis of AFP; protection of AFP from degradation; cellular internalization of AFP, where AFP can intersect with the secretory and/or endolysosomal compartments to inhibit the activities of MHC class I-related molecules involved in antigen presentation or cross-presentation of peptide, carbohydrate, lipid and/or metabolite antigens; inhibition of T cell stimulation by AFP by binding to a MHC Class I-related molecule on the cell surface and inhibiting its ability to bind to a cognate receptor, such as the T cell receptor or a non-cognate receptor, such as a killer inhibitory receptor; or affecting membrane distribution of the MHC class I related molecule. These effects would result in reduced innate and adaptive immunity, such as impacting T cell stimulation by primed dendritic cells and leading to, for example, decreased helper, cytotoxic and humoral immune responses thus reversing AFP-mediated inhibition of anti-tumor responses, for example.

Exemplary inhibitors of AFP and β2M contemplated for use in the various aspects and embodiments described herein include, but are not limited to, antibodies or antigen-binding fragments thereof that specifically bind to an AFP and/or β2M interface comprising two or more amino acid residues, or one or more amino acid residues or epitopes on AFP and/or one or more amino acid residues or epitopes on β2M involved in the binding and/or interactions of AFP and β2M, and inhibit/reduce/block AFP and β2M interactions and/or binding; small molecule agents that specifically bind to an AFP and/or β2M interface comprising two or more amino acid residues, or target or specifically bind one or more amino acid residues on AFP and/or β2M involved in the binding and/or interactions of AFP and β2M, and inhibit/reduce/block AFP and β2M interactions and/or binding; RNA or DNA aptamers that bind to AFP and/or β2M and inhibit/reduce/block AFP and β2M interactions and/or binding; and/or AFP fragments or fusion polypeptides thereof that block endogenous AFP interactions with β2M.

In some embodiments of these aspects and all such aspects described herein, the inhibitor of AFP-β2M interactions inhibits interaction of β2M with an interface of AFP comprising amino acids 105-112 and 131-138 of SEQ ID NO: 1, an interface of AFP comprising amino acids 440-453 of SEQ ID NO: 1, an interface of AFP comprising amino acids 483-493 of SEQ ID NO: 1, an interface of AFP comprising amino acids 519-560 of SEQ ID NO: 1, or any combination thereof.

In some embodiments of these aspects and all such aspects described herein, the inhibition of binding between AFP and β2M further inhibits or prevents interaction or complex formation between β2M and an MHC Class I-related molecule. In some such embodiments, the MHC Class I-related molecule is selected from HFE, HLA-A, HLA-GG, HLA-E, HLA-B, MR1, CD1D, HLA-C, ZA2G, CD1A, and CD1B.

Also provided herein, in some aspects, are compositions, such as pharmaceutical compositions, comprising inhibitors of AFP and MHC Class I-related molecule interactions and a pharmaceutically acceptable carrier, where such inhibitors directly inhibit binding between alpha-fetoprotein (AFP) and one or more MHC Class I-related molecules by interacting with an AFP and/or MHC Class I-related molecule interface. In particular, in some embodiments of the aspects described herein, such inhibitors of AFP and MHC Class I-related molecule interactions can be used to inhibit or block the interacting interfaces between AFP and MHC Class I-related molecules.

As used herein, the terms “AFP-MHC Class I-related molecule inhibitor” and “alpha fetoprotein and MHC Class I-related interactions inhibitor,” or “inhibitor of AFP-MHC Class I-related interactions,” refer to a molecule or agent that significantly blocks, inhibits, reduces, or interferes with the interaction between AFP and one or more MHC Class I-related molecules, and their resultant biological or functional activity in vitro, in situ, and/or in vivo, including docking of MHC Class-I related molecules with β2M, activity of downstream pathways mediated by AFP binding to β2M and signaling. These include, for example, transcytosis of AFP; protection of AFP from degradation; cellular internalization of AFP, where AFP can intersect with the secretory and/or endolysosomal compartments to inhibit the activities of MHC class I-related molecules involved in antigen presentation or cross-presentation of peptide, carbohydrate, lipid and/or metabolite antigens; inhibition of T cell stimulation by AFP by binding to a MHC Class I-related molecule on the cell surface and inhibiting its ability to bind to a cognate receptor such as the T cell receptor or a non-cognate receptor, such as killer inhibitory receptor; or affecting membrane distribution of the MHC Class I-related molecule. These effects would result in reduced innate and adaptive immunity, such as impacting T cell stimulation by primed dendritic cells and leading to, for example, decreased helper, cytotoxic and humoral immune responses thus reversing AFP-mediated inhibition of anti-tumor responses, for example. Exemplary inhibitors of AFP-MHC Class I-related interactions contemplated for use in the various aspects and embodiments described herein include, but are not limited to, antibodies or antigen-binding fragments thereof that specifically bind to one or more amino acid residues or epitopes on AFP and/or one or more MHC Class I-related molecules involved in the binding and/or interactions of AFP and an MHC Class I-related molecule, and inhibit/reduce/block AFP and MHC Class I-related molecule interactions and/or binding; small molecule agents that target or specifically bind one or more amino acid residues on AFP and/or one or more MHC Class I-related molecules involved in the binding and/or interactions of AFP and one or more MHC Class I-related molecules, and inhibit/reduce/block AFP and MHC Class I-related molecule interactions and/or binding; RNA or DNA aptamers that bind to AFP and/or one or more MHC Class I-related molecules and inhibit/reduce/block AFP and one or more MHC Class I-related molecules interactions and/or binding; and/or AFP fragments or fusion polypeptides thereof that block endogenous AFP interactions with one or more MHC Class I-related molecules.

In some embodiments of these aspects and all such aspects described herein, the inhibitor of AFP and MHC Class I-related molecule interactions inhibits interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

In some embodiments of these aspects and all such aspects described herein, the inhibitor of AFP and MHC Class I-related molecule interactions inhibits interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

In some embodiments of these aspects and all such aspects described herein, the inhibitor of AFP and MHC Class I-related molecule interactions inhibits interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

In some embodiments of these aspects and all such aspects described herein, the inhibitor of AFP and MHC Class I-related molecule interactions inhibits interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

In some embodiments of these aspects and all such aspects described herein, the inhibitor of AFP and MHC Class I-related molecule interactions inhibits interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

In some embodiments of these aspects and all such aspects described herein, the inhibitor of AFP and MHC Class I-related molecule interactions inhibits interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

In some embodiments of these aspects and all such aspects described herein, the inhibitor of AFP and MHC Class I-related molecule interactions inhibits interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

In some embodiments of these aspects and all such aspects described herein, the inhibitor of AFP and MHC Class I-related molecule interactions inhibits interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

In some embodiments of these aspects and all such aspects described herein, the inhibitor of AFP and MHC Class I-related molecule interactions inhibits interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

In some embodiments of these aspects and all such aspects described herein, the inhibitor of AFP and MHC Class I-related molecule interactions inhibits interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

In some embodiments of these aspects and all such aspects described herein, the inhibitor of AFP and MHC Class I-related molecule interactions inhibits interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

In some embodiments of these aspects and all such aspects described herein, the inhibitor of AFP and MHC Class I-related molecule interactions inhibits interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

In some embodiments of these aspects and all such aspects described herein, the inhibitor of AFP and MHC Class I-related molecule interactions inhibits interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the inhibitor of AFP and MHC Class I-related molecule interactions inhibits interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

In some embodiments of these aspects and all such aspects described herein, the inhibitor of AFP and MHC Class I-related molecule interactions inhibits interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

In some embodiments of these aspects and all such aspects described herein, the inhibitor of AFP and MHC Class I-related molecule interactions inhibits interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

In some embodiments of these aspects and all such aspects described herein, the inhibitor of AFP and MHC Class I-related molecule interactions also inhibits binding between S527 or D528 of SEQ ID NO: 2 and E50 and 67Y of SEQ ID NO: 2, respectively, complexed with an MHC Class I-related molecule.

In some embodiments of these aspects and all such aspects described herein, the inhibitor of AFP and MHC Class I-related molecule interactions also inhibits binding between R604 of AFP and the carbonyl oxygen at E50 of β2M, wherein the β2M is complexed with an MHC Class I-related molecule.

As used herein, in regard to both the inhibitors of AFP-β2M interactions and inhibitors of AFP and MHC Class I-related molecule interactions, such inhibitors have the ability to reduce or decrease the interaction between AFP and β2M or AFP and a given MHC Class I-related molecule and/or their resultant biological or functional activity in vitro, in situ, and/or in vivo by at least 5%, at least 10%, at least 20%, at least 30%, at least 40%, at least 50%, at least 60%, at least 70%, at least 80%, at least 90%, at least 95%, at least 98%, at least 99%, or more, relative to the interaction and/or activity in the absence of the inhibitor.

The terms “decrease,” “decreased,” “decreasing,” “reduce,” “reduced,” “reducing,” “inhibit,” inhibiting,” and “inhibited,” as used interchangeably herein, when used in regard to the interactions between AFP and β2M or interactions between AFP and MHC Class I-related molecules, generally mean either reducing or inhibiting the interaction between or binding of AFP and β2M and/or AFP and an MHC Class I-related molecule by at least 5%, at least 10%, at least 25%, at least 50%, at least 60%, at least 70%, at least 80%, at least 90%, at least 95%, at least 98%, or more, compared to the interaction between AFP and β2M and/or AFP and the MHC Class I-related molecule under the same conditions, but in the absence of the inhibitors described herein.

In some embodiments of the compositions, methods, and uses described herein, the MHC Class I-related molecule is selected from HFE, HLA-A, HLA-GG, HLA-E, HLA-B, MR1, CD1D, HLA-C, ZA2G, CD1A and CD1B.

In some embodiments of the compositions, methods, and uses described herein, the inhibitor of AFP-β2M interactions or the inhibitor of AFP and an MHC Class I-related molecule interaction is an antibody or antigen-binding fragment thereof. In some embodiments of the aspects described herein, such inhibitors of AFP and an MHC Class I-related molecule interaction can be used to inhibit or block the AFP binding site or interface on the MHC Class I-related molecule, as described herein. In some embodiments, an antibody or antigen-binding fragment inhibitor of AFP and an MHC Class I-related molecule interaction binds to an epitope that comprises the AFP binding site on the MHC Class I-related molecule.

Antibodies or antigen-binding fragments thereof that are specific for or that selectively bind AFP, β2M, an MHC Class I-related molecule, AFP bound to β2M, and/or AFP bound to an MHC Class I-related molecule, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Examples of antibody fragments encompassed by the terms antibody fragment or antigen-binding fragment as described herein include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the FAT fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}—C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

With respect to a target or antigen, the term “ligand interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerization (such as homodimerization or heterodimerization) of the target or antigen; or any other site, epitope, antigenic determinant, part, domain or stretch of amino acid residues on the target or antigen that is involved in a biological action or mechanism of the target or antigen, i.e., AFP, β2M, an MHC Class I-related molecule, AFP bound to β2M, or AFP bound to an MHC Class I-related molecule. For example, in some embodiments, a ligand interaction site on AFP can be any site on AFP to which β2M or an MHC Class I-related molecule selected from HFE, HLA-A, HLA-GG, HLA-E, HLA-B, MR1, CD1D, HLA-C, ZA2G, CD1A and CD1B binds or interacts, and, for example, affects the availability or conformation of the binding sites for β2M or MHC Class I-related molecules and/or other AFP binding molecules, within the AFP, β2M and MHC Class I-related molecule multimeric complex. More generally, a “ligand interaction site” can be any site, epitope, antigenic determinant, part, domain or stretch of amino acid residues on a target or antigen to which an inhibitor of AFP and β2M interactions or AFP and MHC Class I-related molecule interactions described herein can bind such that the interaction or binding between AFP and β2M and/or AFP and an MHC Class I-related molecule (and/or any pathway, interaction, signaling, biological mechanism or biological effect mediated by AFP binding to β2M or an MHC Class I-related molecule is involved) is modulated.

In the context of an antibody or antigen-binding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}. As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10,000 times or more better than the affinity with which said amino acid sequence or polypeptide binds to another target or polypeptide. Preferably, when an antibody or antigen-binding fragment thereof is “specific for” a target or antigen, compared to another target or antigen, it is directed against said target or antigen, but not directed against another target or antigen.

However, as understood by one of ordinary skill in the art, in some embodiments, where a binding site on a target is shared or partially shared by multiple, different ligands, an antibody or antigen binding fragment thereof can specifically bind to a target, such as AFP, and have the functional effect of inhibiting/preventing binding of multiple, different ligands, such as FcRn, and/or one or more MHC Class I-related molecules.

Avidity is a measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D}) of 10⁻⁵ to 10⁻¹² moles/liter or less, and preferably 10⁻⁷ to 10⁻¹² moles/liter or less, and more preferably, 10⁻⁸ to 10⁻¹² moles/liter (i.e. with an association constant (K_{A}) of 10⁵ to 10¹² liter/moles or more, and preferably 10⁷ to 10¹² liter/moles or more, and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on an antibody or antigen-binding fragment inhibitor of AFP-β2M or inhibitor of AFP and MHC Class I-related molecule interactions described herein will bind to AFP, β2M, and/or an MHC Class I-related molecule with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

In some embodiments of the compositions, methods, and uses described herein, the inhibitor of AFP-β2M interactions is a monoclonal antibody.

In some embodiments of the compositions, methods, and uses described herein, the inhibitor of AFP and MHC Class I-related interactions is a monoclonal antibody.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each antibody in a monoclonal preparation is directed against the same, single determinant on the antigen. It is to be understood that the term “monoclonal antibody” refers to an antibody that is derived from a single clone, including any eukaryotic, prokaryotic, or phage clone, and not the method by which it is produced. The term “monoclonal antibody” as used herein is not limited to antibodies produced through hybridoma technology, and the modifier “monoclonal” is not to be construed as requiring production of the antibody by any particular method. For example, monoclonal antibodies to be used in accordance with the invention can be made by the hybridoma method first described by Kohler et al., Nature 256:495 (1975), or later adaptations thereof, or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

In some embodiments of the compositions, methods, and uses described herein, the inhibitor of AFP and β2M interactions is a chimeric antibody derivative of an antibody or antigen-binding fragment thereof that binds AFP, β2M, and/or AFP bound to β2M.

In some embodiments of the compositions, methods, and uses described herein, the inhibitor of AFP and MHC Class I-related molecule interactions is a chimeric antibody derivative of an antibody or antigen-binding fragment thereof that binds AFP, an MHC Class I-related molecule, and/or AFP bound to an MHC Class I-related molecule and/or β2M.

As used herein, the term “chimeric antibody” refers to an antibody molecule in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the desired antigen, e.g., AFP and/or FcRn. See, for example, Takeda et al., 1985, Nature 314:452; Cabilly et al., U.S. Pat. No. 4,816,567; Boss et al., Tanaguchi et al., European Patent Publication EP171496; European Patent Publication 0173494, United Kingdom patent GB 2177096B).

In some embodiments of the compositions, methods, and uses described herein, the inhibitor of AFP and β2M interactions is a humanized antibody derivative of an antagonist antibody or antigen-binding fragment thereof that binds AFP, β2M, and/or AFP bound to β2M.

In some embodiments of the compositions, methods, and uses described herein, the inhibitor of AFP and MHC Class I-related molecule interactions is a humanized antibody derivative of an antagonist antibody or antigen-binding fragment thereof that binds AFP, an MHC Class I-related molecule, and/or AFP bound to an MHC Class I-related molecule and/or β2M.

Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments of the compositions, methods, and uses comprising any of the antibodies or antigen-binding fragments thereof inhibitors of AFP-β2M or inhibitors of AFP and MHC Class I-related molecule interactions described herein, the inhibitor antibody or antigen-binding fragment is an antibody derivative. For example, but not by way of limitation, antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids, or alternative scaffolds such as centyrins, DARPINS, or fynomers engineered to bind AFP, β2M, and/or an MHC Class I-related molecule and inhibit their interactions.

The inhibitor antibodies and antigen-binding fragments thereof described herein can be generated by any suitable method known in the art. Monoclonal and polyclonal antibodies against, for example, FcRn, are known in the art. To the extent necessary, e.g., to generate antibodies with particular characteristics or epitope specificity, the skilled artisan can generate new monoclonal or polyclonal antibody inhibitors of AFP-β2M or inhibitors of AFP and MHC Class I-related molecule interactions as briefly discussed herein or as known in the art.

Polyclonal antibodies can be produced by various procedures well known in the art. For example, AFP, β2M, an MHC Class I-related molecule, or fragments thereof comprising one or more of the AFP, β2M, and/or MHC Class I-related molecule interaction sites or interfaces, for example, can be administered to various host animals including, but not limited to, rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R¹N═C═NR, where R and R¹ are different alkyl groups. Various other adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Suitable adjuvants are also well known to one of skill in the art.

Monoclonal antibodies can be prepared using a wide variety of techniques known in the art including the use of hybridoma, recombinant, and phage display technologies, or a combination thereof. Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

In some embodiments of the compositions, methods, and uses described herein, completely human antibodies are used as inhibitors of AFP-β2M interactions or inhibitors of AFP and MHC Class I-related molecule interactions, which are particularly desirable for the therapeutic treatment of human patients.

Human antibodies can be made by a variety of methods known in the art, including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992), the contents of which are herein incorporated by reference in their entireties. Alternatively, phage display technology (McCafferty et al., Nature 348:552-553 (1990)) can be used to produce human antibodies and antibody fragments in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

“An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the human heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol. Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., AFP, β2M, an MHC Class I-related molecule, AFP bound to β2M, and/or AFP bound to an MHC Class I-related molecule, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The “Fab” fragment contains a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}—C_{H}1-V_{H}-C_{H}1) which, together with complementary light chain polypeptides, form a pair of antigen binding regions. Linear antibodies can be bispecific or monospecific.

Various techniques have been developed for the production of antibody or antigen-binding fragments. The antibodies described herein can be fragmented using conventional techniques and the fragments screened for utility in the same manner as described above for the whole antibodies. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). For example, Fab and F(ab′)₂ fragments of the bispecific and multispecific antibodies described herein can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain. However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including the in vivo use of antibodies in humans as described herein and in vitro proliferation or cytotoxicity assays, it is preferable to use chimeric, humanized, or human antibodies.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary.

Complementary domains can be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

In some embodiments of the compositions, methods, and uses described herein, the inhibitor of AFP-β2M interactions or the inhibitor of AFP and MHC Class I-related molecule interactions is a small molecule inhibitor, agent, or compound. In some embodiments of the aspects described herein, such small molecule inhibitors can be used to inhibit or block the AFP binding site or interface on β2M, inhibit or block αβ2M binding site or interface on AFP, inhibit or block the AFP binding site or interface on the MHC Class I molecule, or can be used to inhibit or block an MHC Class I-related molecule binding site or interface on AFP, as described herein.

Such small molecule inhibitors include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

Inhibitors of AFP-β2M interactions and inhibitors of AFP and MHC Class I-related molecule interactions for use in the compositions, methods, and uses described herein can be identified or characterized using methods known in the art, such as protein-protein binding assays, biochemical screening assays, immunoassays, and cell-based assays, which are well known in the art, including, but not limited to, those described herein in the Examples and Figures.

For example, to identify a molecule that inhibits interaction between AFP and β2M or AFP and MHC Class I-related molecules, relevant interacting molecules will be evaluated by their ability to interrupt the interactions by biophysical assays such as surface plasmon resonance, biochemical methods such as competitive ELISA or radioimmunoassay or cell-based assays, such as competition studies using, for example, flow cytometry on native or transfected cells.

Also provided herein, in some aspects, are compositions, such as pharmaceutical compositions, comprising potentiators of AFP and β2M interactions and AFP and MHC Class I-related molecule interactions. Such potentiators are used to enhance/increase/potentiate the interaction between AFP and β2M or AFP and MHC Class I-related molecules, thereby increasing immunosuppressive activities of AFP in the treatment of disorders and conditions in need of enhanced AFP levels, including autoimmune disorders, transplant patients, and high-risk pregnancies, for example. These potentiators can modulate pathways involving, for example, docking of MHC Class I-related molecules with β2M, and activity of downstream pathways mediated by AFP binding to β2M and signaling. These include, for example: transcytosis of AFP; AFP degradation; cellular internalization of AFP, where AFP can intersect with the secretory and/or endolysosomal compartments to modulate the activities of MHC class I-related molecules involved in antigen presentation or cross-presentation of peptide, carbohydrate, lipid and/or metabolite antigens; T cell stimulation by AFP by binding to a MHC Class I-related molecule on the cell surface and impacting its ability to bind to a cognate receptor, such as the T cell receptor or a non-cognate receptor, such as a killer inhibitory receptor; or affecting membrane distribution of the MHC class I related molecule. These effects would result in modulation of innate and adaptive immunity, such as impacting T cell stimulation by primed dendritic cells and leading to, for example, altered helper, cytotoxic, and humoral immune responses, for example.

As used herein, the terms “AFP-β2M potentiator,” “potentiator of AFP-β2M interactions,” AFP-β2M activator agent,” and “AFP-β2M agonist agent” refer to a molecule or agent that mimics or up-regulates (e.g., increases, potentiates or supplements) the biological activity of AFP binding to β2M in vitro, in situ, and/or in vivo, including downstream pathways mediated by AFP binding to β2M and signaling, such as, for example, docking of MHC Class I-related molecules with β2M, activity of downstream pathways mediated by AFP binding to β2M and signaling. These include, for example, transcytosis of AFP; AFP degradation; cellular internalization of AFP, where AFP can intersect with the secretory and/or endolysosomal compartments to modulate the activities of MHC class I-related molecules involved in antigen presentation or cross-presentation of peptide, carbohydrate, lipid and/or metabolite antigens; T cell stimulation by AFP by binding to a MHC Class I-related molecule on the cell surface and impacting its ability to bind to a cognate receptor, such as the T cell receptor or a non-cognate receptor, such as killer inhibitory receptor; or affecting membrane distribution of the MHC class I related molecule. These effects would result in modulation of innate and adaptive immunity, such as impacting T cell stimulation by primed dendritic cells and leading to, for example, altered helper, cytotoxic, and humoral immune responses, for example.

An AFP-β2M potentiator or agonist can be, in some embodiments, an AFP protein fragment or derivative thereof having at least one bioactivity of the wild-type AFP. An AFP-β2M potentiator can also be a compound which increases the interaction of AFP with β2M, for example, in enabling immunosuppression in the treatment of autoimmunity as in multiple sclerosis, rheumatoid arthritis or inflammatory bowel disease for example or in enabling tissue regeneration as associated for example with the liver during transplantation or resection. Exemplary AFP-β2M potentiators or agonists contemplated for use in the various aspects and embodiments described herein include, but are not limited to, antibodies or antigen-binding fragments thereof that specifically bind to AFP bound to β2M and enhance the interaction; RNA or DNA aptamers that bind to β2M and mimic AFP binding to β2M; AFP structural analogs or AFP functional fragments, derivatives, or fusion polypeptides thereof; and small molecule agents that target or bind to β2M and act as functional mimics of AFP binding to β2M.

As used herein, the terms “AFP-MHC Class I-related molecule potentiator,” “potentiator of AFP-MHC Class I-related molecule interactions,” AFP-MHC Class I-related molecule activator agent,” and “AFP-MHC Class I-related molecule agonist agent” refer to a molecule or agent that mimics or up-regulates (e.g., increases, potentiates or supplements) the biological activity of AFP binding to an MHC Class I-related molecule in vitro, in situ, and/or in vivo, including downstream pathways mediated by AFP binding to a MHC Class I-related molecule and signaling, such as, for example, transcytosis of AFP, inhibition of T cell stimulation by immune complex-primed dendritic cells, AFP-mediated inhibition of immune responses, and/or increased serum half-life of AFP. An AFP-MHC Class I-related molecule interaction potentiator or agonist can be, in some embodiments, an AFP protein fragment or derivative thereof having at least one bioactivity of the wild-type AFP. An AFP-MHC Class I-related molecule interaction potentiator can also be a compound which increases the interaction of AFP with an MHC Class I-related molecule, for example. Exemplary AFP-MHC Class I-related molecule interaction potentiators or agonists contemplated for use in the various aspects and embodiments described herein include, but are not limited to, antibodies or antigen-binding fragments thereof that specifically bind to AFP bound to an MHC Class I-related molecule and enhance the interaction; RNA or DNA aptamers that bind to β2M and mimic MHC Class I-related molecule binding to β2M; AFP structural analogs or AFP functional fragments, derivatives, or fusion polypeptides thereof; and small molecule agents that target or bind to an MHC Class I-related molecule and act as functional mimics of AFP binding to the MHC Class I-related molecule.

As used herein, an AFP-β2M potentiator or an AFP-MHC Class I-related molecule potentiator has the ability to increase or enhance the activity of AFP binding to β2M or to an MHC Class I-related molecule or mimic/replicate the downstream functional consequences mediated by AFP binding to β2M or an MHC Class I-related molecule by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the potentiator.

In some embodiments of these aspects and all such aspects described herein, the AFP-β2M potentiator increases interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO: 4, an interface of β2M comprising amino acids 81-96 of SEQ ID NO: 4, or any combination thereof.

In some embodiments of these aspects and all such aspects described herein, the AFP-β2M potentiator increases interaction of β2M with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

In some embodiments of these aspects and all such aspects described herein, the AFP-MHC Class I-related molecule potentiator increases interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

In some embodiments of these aspects and all such aspects described herein, the AFP-MHC Class I-related molecule potentiator increases interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

In some embodiments of these aspects and all such aspects described herein, the AFP-MHC Class I-related molecule potentiator increases interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

In some embodiments of these aspects and all such aspects described herein, the AFP-MHC Class I-related molecule potentiator increases interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

In some embodiments of these aspects and all such aspects described herein, the AFP-MHC Class I-related molecule potentiator increases interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

In some embodiments of these aspects and all such aspects described herein, the AFP-MHC Class I-related molecule potentiator increases interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

In some embodiments of these aspects and all such aspects described herein, the AFP-MHC Class I-related molecule potentiator increases interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

In some embodiments of these aspects and all such aspects described herein, the AFP-MHC Class I-related molecule potentiator increases interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

In some embodiments of these aspects and all such aspects described herein, the AFP-MHC Class I-related molecule potentiator increases interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

In some embodiments of these aspects and all such aspects described herein, the AFP-MHC Class I-related molecule potentiator increases interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

In some embodiments of these aspects and all such aspects described herein, the AFP-MHC Class I-related molecule potentiator increases interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

In some embodiments of these aspects and all such aspects described herein, the AFP-MHC Class I-related molecule potentiator increases interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

In some embodiments of these aspects and all such aspects described herein, the AFP-MHC Class I-related molecule potentiator increases interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

In some embodiments of these aspects and all such aspects described herein, the AFP-MHC Class I-related molecule potentiator increases interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

In some embodiments of these aspects and all such aspects described herein, the AFP-MHC Class I-related molecule potentiator increases interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

In some embodiments of these aspects and all such aspects described herein, the AFP-MHC Class I-related molecule potentiator increases interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

In some embodiments of these aspects and all such aspects described herein, the AFP-MHC Class I-related molecule potentiator increases also increases binding between S527 or D528 of AFP and E50 and 67Y of β2M, respectively, complexed with an MHC Class I-related molecule.

In some embodiments of these aspects and all such aspects described herein, the AFP-MHC Class I-related molecule potentiator increases also increases binding between R604 of AFP and the carbonyl oxygen at E50 of β2M, wherein the β2M is complexed with an MHC Class I-related molecule.

In some embodiments of the compositions, methods, and uses described herein, the AFP-β2M potentiator or the AFP-MHC Class I-related molecule potentiator is an antibody or antigen-binding fragment thereof that selectively binds or physically interacts with AFP bound to β2M or an MHC Class I-related molecule and enhances the interaction of AFP and β2M or AFP and an MHC Class I-related molecule, thereby resulting in increased transcytosis of AFP, increased inhibition of T cell stimulation by immune complex-primed dendritic cells, increased AFP-mediated inhibition of immune responses, and/or increased serum half-life of AFP. Exemplary assays to measure increases or up-regulation of downstream pathway activities are known to those of ordinary skill in the art and are provided herein in the Examples.

In some embodiments of the compositions, methods, and uses described herein, the AFP-β2M potentiator or the AFP-MHC Class I-related molecule potentiator is a monoclonal antibody. In some embodiments of the compositions, methods, and uses described herein, the AFP-β2M potentiator or the AFP-MHC Class I-related molecule potentiator is an antibody fragment or antigen-binding fragment, as the term is described elsewhere herein.

In some embodiments of the compositions, methods, and uses described herein, the AFP-β2M potentiator or the AFP-MHC Class I-related molecule potentiator is a chimeric antibody derivative of the AFP-β2M potentiator or the AFP-MHC Class I-related molecule potentiator antibodies and antigen-binding fragments thereof, as the term is described elsewhere herein.

In some embodiments of the compositions, methods, and uses described herein, the AFP-β2M potentiator or the AFP-MHC Class I-related molecule potentiator is a humanized antibody derivative, as the term is described elsewhere herein.

In some embodiments, the AFP-β2M potentiator or the AFP-MHC Class I-related molecule potentiator antibodies and antigen-binding fragments thereof described herein include derivatives that are modified, e.g., by the covalent attachment of any type of molecule to the antibody, provided that covalent attachment does not prevent the antibody from binding to the target antigen.

In some embodiments, the AFP-β2M potentiator or the AFP-MHC Class I-related molecule potentiator antibodies and antigen-binding fragments thereof described herein are completely human antibodies or antigen-binding fragments thereof, which are particularly desirable for the therapeutic treatment of human patients. Human antibodies can be made by a variety of methods known in the art, and as described in more detail elsewhere herein.

The AFP-β2M potentiator or the AFP-MHC Class I-related molecule potentiator antibodies and antigen-binding fragments thereof described herein, as well as any of the other antibodies or antigen-binding fragments thereof described herein in various aspects and embodiments, can be generated by any suitable method known in the art.

In some embodiments of the compositions, methods, and uses described herein, the AFP-β2M potentiator or the AFP-MHC Class I-related molecule potentiator is a small molecule potentiator, activator, or agonist, including, but not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, about 1000 to about 10,000 Da.

In some embodiments of the compositions, methods, and uses described herein, the AFP-β2M potentiator or the AFP-MHC Class I-related molecule potentiator is an RNA or DNA aptamer that binds or physically interacts with AFP, β2M, or an MHC Class I-related molecule, and enhances or promotes interactions between AFP and β2M or AFP and an MHC Class I-related molecule.

In some embodiments of the compositions, methods, and uses described herein, the AFP-β2M potentiator or the AFP-MHC Class I-related molecule potentiator comprises an AFP structural analog, functional fragment, or derivative, such as an AFP variant engineered to possess increased binding to β2M or to the MHC Class I-related molecule. The term “AFP structural analog,” “AFP functional fragment,” or “AFP derivative” as used herein, refer to compounds, such as peptides, that can bind to β2M or to an MHC Class I-related molecule under physiological conditions in vitro or in vivo, wherein the binding at least partially mimics or increases a biological activity normally mediated by endogenous binding. Suitable AFP structural analogs, functional fragments, or derivatives can be designed and synthesized through molecular modeling of AFP binding to β2M or to an MHC Class I-related molecule, for example.

AFP-β2M potentiators and AFP-MHC Class I-related molecule potentiators for use in the compositions, methods, and uses described herein can be identified or characterized using methods known in the art, such as protein-protein binding assays, biochemical screening assays, immunoassays, and cell-based assays, which are well known in the art, such as those described herein in the Examples.

For the clinical use of the methods and uses described herein, administration of the compositions comprising inhibitors of AFP-β2M interactions, inhibitors of MHC Class I-related molecule interactions, AFP-β2M potentiators, or AFP-MHC Class I-related molecule potentiators can include formulation into pharmaceutical compositions or pharmaceutical formulations for parenteral administration, e.g., intravenous; mucosal, e.g., intranasal; ocular, or other mode of administration. In some embodiments, the inhibitors of AFP-β2M interactions, inhibitors of MHC Class I-related molecule interactions, AFP-β2M potentiators, or AFP-MHC Class I-related molecule potentiators described herein, can be administered along with any pharmaceutically acceptable carrier compound, material, or composition which results in an effective treatment in the subject. Thus, a pharmaceutical formulation for use in the methods described herein can contain an inhibitor of AFP-β2M interactions, inhibitor of MHC Class I-related molecule interactions, AFP-β2M potentiator, or AFP-MHC Class I-related molecule potentiator as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of inhibitors of AFP-β2M interactions, inhibitors of MHC Class I-related molecule interactions, AFP-β2M potentiators, or AFP-MHC Class I-related molecule potentiators. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The inhibitors of AFP-β2M interactions, inhibitors of MHC Class I-related molecule interactions, AFP-β2M potentiators, or AFP-MHC Class I-related molecule potentiators described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, the inhibitors of AFP-β2M interactions, inhibitors of MHC Class I-related molecule interactions, AFP-β2M potentiators, or AFP-MHC Class I-related molecule potentiators can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of the compositions comprising inhibitors of AFP-β2M interactions, inhibitors of MHC Class I-related molecule interactions, AFP-β2M potentiators, or AFP-MHC Class I-related molecule potentiators that can be used in the methods described herein are described below.

Parenteral Dosage Forms.

Parenteral dosage forms of the inhibitors of AFP-β2M interactions, inhibitors of MHC Class I-related molecule interactions, AFP-β2M potentiators, or AFP-MHC Class I-related molecule potentiators can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol Formulations.

Inhibitors of AFP-β2M interactions, inhibitors of MHC Class I-related molecule interactions, AFP-β2M potentiators, or AFP-MHC Class I-related molecule potentiators can be packaged in a pressurized aerosol container together with suitable propellants, for example, hydrocarbon propellants like propane, butane, or isobutane with conventional adjuvants. Inhibitors of AFP-β2M interactions, inhibitors of MHC Class I-related molecule interactions, AFP-β2M potentiators, or AFP-MHC Class I-related molecule potentiators described herein, can also be administered in a non-pressurized form such as in a nebulizer or atomizer. Inhibitors of AFP-β2M interactions, inhibitors of MHC Class I-related molecule interactions, AFP-β2M potentiators, or AFP-MHC Class I-related molecule potentiators described herein can also be administered directly to the airways in the form of a dry powder, for example, by use of an inhaler.

Suitable powder compositions include, by way of illustration, powdered preparations of inhibitors of AFP-β2M interactions, inhibitors of MHC Class I-related molecule interactions, AFP-β2M potentiators, or AFP-MHC Class I-related molecule potentiators described herein, thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the inhibitors of AFP-β2M interactions, inhibitors of MHC Class I-related molecule interactions, AFP-β2M potentiators, or AFP-MHC Class I-related molecule potentiators described herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms.

In some embodiments of the aspects described herein, inhibitors of AFP-β2M interactions, inhibitors of MHC Class I-related molecule interactions, AFP-β2M potentiators, or AFP-MHC Class I-related molecule potentiators can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness inhibitors of AFP-β2M interactions, inhibitors of MHC Class I-related molecule interactions, AFP-β2M potentiators, or AFP-MHC Class I-related molecule potentiators is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the inhibitors of AFP-β2M interactions, inhibitors of MHC Class I-related molecule interactions, AFP-β2M potentiators, or AFP-MHC Class I-related molecule potentiators described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B 1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the methods described herein, inhibitors of AFP-β2M interactions, inhibitors of MHC Class I-related molecule interactions, AFP-β2M potentiators, or AFP-MHC Class I-related molecule potentiators for use in the methods described herein are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the inhibitors of AFP-β2M interactions, inhibitors of MHC Class I-related molecule interactions, AFP-β2M potentiators, or AFP-MHC Class I-related molecule potentiators described herein administered over the course of treatment to the subject or patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Methods of Treatment and Uses

As demonstrated herein, alpha-fetoprotein (AFP) can bind both β2M independently, as well as various members of the family of MHC Class I-related molecules using distinct binding interfaces, as described herein. Accordingly, provided herein, in some aspects, are methods to inhibit or reduce interactions of AFP and β2M, interactions of AFP and MHC Class I-related molecules and/or interactions of AFP and β2M and MHC Class I-related molecules in diseases or disorders where elevated AFP levels are associated with immunosuppression. Such methods comprise administering a therapeutically effective amount of a pharmaceutical composition comprising any of the inhibitors of AFP-β2M interactions and/or inhibitors of AFP-MHC Class I-related molecule interactions described herein to a subject in need thereof.

In some embodiments of these aspects and all such aspects described herein, a subject having a disease or disorder associated with elevated AFP levels has or has been diagnosed with cancer.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In some embodiments of these methods and all such methods described herein, the methods further comprise administering an anti-cancer therapy or agent to a subject in addition to the inhibitors of AFP-β2M interactions and/or inhibitors of AFP-MHC Class I-related molecule interactions described herein.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX2 inhibitor (e.g., celecoxib), interferons, cytokines, antagonists (e.g., neutralizing antibodies) that bind to one or more of the following targets PD1, PDL1, PDL2, TIM3 or any TIM family member, CEACAM1 or any CEACAM family member, ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

In some embodiments, an anti-cancer therapy comprises an immunotherapy such as adoptive cell transfer. “Adoptive cell transfer,” as used herein, includes immunotherapies involving genetically engineering a subject or patient's own T cells to produce special receptors on their surface called chimeric antigen receptors (CARs). CARs are proteins that allow the T cells to recognize a specific protein (antigen) on tumor cells. These engineered CAR T cells are then grown in the laboratory until they number in the billions. The expanded population of CAR T cells is then infused into the patient. After the infusion, the T cells multiply in the subject's body and, with guidance from their engineered receptor, recognize and kill cancer cells that harbor the antigen on their surfaces.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including active fragments and/or variants thereof.

In some embodiments of these methods and all such methods described herein, the methods further comprise administering a chemotherapeutic agent to the subject being administered the inhibitors of AFP-β2M interactions and/or inhibitors of AFP-MHC Class I-related molecule interactions described herein.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In some embodiments of these methods and all such methods described herein, the methods further comprise administering a tumor or cancer antigen to a subject being administered the inhibitors of AFP-β2M interactions and/or inhibitors of AFP-MHC Class I-related molecule interactions described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the symptoms of the disorder being treated, the presence or size of metastases or micrometastases, the size of the primary tumor, the presence or the size of the dormant tumor, etc.

As used herein, “alleviating a symptom of a cancer or tumor” is ameliorating any condition or symptom associated with the cancer such as the symptoms of the cancer being treated, the presence or size of metastases or micrometastases, the size of the primary tumor, the presence or the size of the dormant tumor, etc. As compared with an equivalent untreated control, such as a subject prior to the administration of the AFP-FcRn inhibitors, such reduction or degree of prevention is at least 5%, 10%, 20%, 40%, 50%, 60%, 80%, 90%, 95%, or more as measured by any standard technique known to one of ordinary skill in the art. A patient or subject who is being treated for a cancer or tumor is one who a medical practitioner has diagnosed as having such a condition. Diagnosis can be by any suitable means.

Also provided herein, in some aspects, are methods to increase or potentiate interactions of AFP with β2M, interactions of AFP with MHC Class I-related molecules, or interactions of AFP with both β2M and MHC Class I-related molecules in diseases or disorders associated with decreased AFP levels or where increasing AFP levels is beneficial comprising administering a therapeutically effective amount of a pharmaceutical composition comprising an AFP-β2M potentiator, an AFP-MHC Class I-related molecule potentiator, or a combination thereof to a subject in need thereof.

In some embodiments of these methods and all such methods described herein, a subject in need of increased AFP levels and/or increased interactions of AFP with β2M and/or interactions of AFP with MHC Class I-related molecules has or has been diagnosed with an autoimmune disease or disorder.

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In some embodiments of the aspects described herein, the autoimmune disease is selected from the group consisting of multiple sclerosis, type-I diabetes, Hashimoto's thyroiditis, Crohn's disease or colitis, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments of these methods and all such methods described herein, a subject in need of increased AFP levels or increased interactions of AFP with β2M and/or interactions of AFP with MHC Class I-related molecules has or has been diagnosed with host versus graft disease (HVGD). In a further such embodiment, the subject being treated with the methods described herein is an organ or tissue transplant recipient. In some embodiments, the methods are used for increasing transplantation tolerance in a subject. In some such embodiments, the subject is a recipient of an allogenic transplant.

The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

In another embodiment, increased interactions of AFP with β2M and/or interactions of AFP with MHC Class I-related molecules is required in enabling tissue regeneration as after transplantation or partial resection of an organ such as in the case of the liver after its replacement or partial resection of a lobe or in the case of organ injury (e.g. hepatitis) wherein tissue regeneration is desired.

As used herein, in regard to any of the compositions, methods, and uses comprising any of the inhibitors of AFP-β2M interactions, inhibitors of MHC Class I-related molecule interactions, AFP-β2M potentiators, or AFP-MHC Class I-related molecule potentiators described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The terms “subject,” “patient,” and “individual” as used in regard to any of the methods described herein are used interchangeably herein, and refer to an animal, for example a human, recipient of the inhibitors described herein. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like. Production mammal, e.g. cow, sheep, pig, and the like are also encompassed in the term subject.

The term “effective amount” as used herein refers to the amount of any of the inhibitors of AFP-β2M interactions, inhibitors of MHC Class I-related molecule interactions, AFP-β2M potentiators, or AFP-MHC Class I-related molecule potentiators described herein, needed to alleviate at least one or more symptom of the disease or disorder being treated, and relates to a sufficient amount of pharmacological composition to provide the desired effect, e.g., increase or decrease serum AFP levels. The term “therapeutically effective amount” therefore refers to an amount of the inhibitors or potentiators described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The inhibitors of AFP-β2M interactions, inhibitors of MHC Class I-related molecule interactions, AFP-β2M potentiators, or AFP-MHC Class I-related molecule potentiators described herein can be administered to a subject in need thereof by any appropriate route which results in an effective treatment in the subject. As used herein, the terms “administering,” and “introducing” are used interchangeably and refer to the placement of an inhibitor of AFP-β2M interactions, inhibitor of MHC Class I-related molecule interactions, AFP-β2M potentiator, or AFP-MHC Class I-related molecule potentiator into a subject by a method or route which results in at least partial localization of such agents at a desired site, such as a tumor site or site of inflammation, such that a desired effect(s) is produced.

In some embodiments, the inhibitors of AFP-β2M interactions, inhibitors of MHC Class I-related molecule interactions, AFP-β2M potentiators, or AFP-MHC Class I-related molecule potentiators described herein can be administered to a subject by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the inhibitors of AFP-β2M interactions, inhibitors of MHC Class I-related molecule interactions, AFP-β2M potentiators, or AFP-MHC Class I-related molecule potentiators, other than directly into a target site, tissue, or organ, such that it enters the subject's circulatory system and, thus, is subject to metabolism and other like processes.

Some embodiments of the technology described herein can be defined according to any of the following numbered paragraphs:

1. A pharmaceutical composition comprising an inhibitor of alpha-fetoprotein (AFP)-β2-microglobulin (β2M) interactions and a pharmaceutically acceptable carrier, wherein said inhibitor of AFP-β2M interactions inhibits binding between AFP and β2M.

2. The pharmaceutical composition of paragraph 1, wherein the inhibitor of AFP-β2M interactions inhibits interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO: 4, an interface of β2M comprising amino acids 81-96 of SEQ ID NO: 4, or any combination thereof.

3. The pharmaceutical composition of paragraph 1, wherein the inhibitor of AFP-β2M interactions inhibits interaction of β2M with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

4. The pharmaceutical composition of paragraph 1, wherein the inhibition of binding between AFP and β2M further inhibits or prevents interaction or complex formation between β2M and an MHC Class I-related molecule.

5. The pharmaceutical composition of paragraph 4, wherein the MHC Class I-related molecule is selected from HFE, HLA-A, HLA-G, HLA-E, HLA-B, MR1, CD1D, HLA-C, ZA2G, CD1A, CD1B.

6. The pharmaceutical composition of any one of paragraphs 1-5, wherein the inhibitor of AFP-β2M interactions is an antibody or antigen-binding fragment thereof, a small molecule compound, or an RNA or DNA aptamer.

7. The pharmaceutical composition of paragraph 6, wherein the antibody or antigen-binding fragment thereof is a chimeric, humanized, or completely human antibody or antigen-binding fragment thereof.

8. A pharmaceutical composition comprising an inhibitor of alpha-fetoprotein (AFP)-MHC Class I-related interactions and a pharmaceutically acceptable carrier, wherein said inhibitor of AFP-MHC Class I-related interactions inhibits binding between AFP and an MHC Class I-related molecule.

9. The pharmaceutical composition of paragraph 8, wherein the MHC Class I-related molecule is selected from HFE, HLA-A, HLA-G, HLA-E, HLA-B, MR1, CD1D, HLA-C, ZA2G, CD1A, and CD1B.

10. The pharmaceutical composition of paragraph 8, wherein the inhibitor of AFP-MHC Class I-related interactions inhibits interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

11. The pharmaceutical composition of paragraph 8, wherein the inhibitor of AFP-MHC Class I-related interactions inhibits interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

12. The pharmaceutical composition of paragraph 8, wherein the inhibitor of AFP-MHC Class I-related interactions inhibits interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

13. The pharmaceutical composition of paragraph 8, wherein the inhibitor of AFP-MHC Class I-related interactions inhibits interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

14. The pharmaceutical composition of paragraph 8, wherein the inhibitor of AFP-MHC Class I-related interactions inhibits interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

15. The pharmaceutical composition of paragraph 8, wherein the inhibitor of AFP-MHC Class I-related interactions inhibits interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

16. The pharmaceutical composition of paragraph 8, wherein the inhibitor of AFP-MHC Class I-related interactions inhibits interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

17. The pharmaceutical composition of paragraph 8, wherein the inhibitor of AFP-MHC Class I-related interactions inhibits interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

18. The pharmaceutical composition of paragraph 8, wherein the inhibitor of AFP and MHC Class I-related molecule interactions inhibits interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

19. The pharmaceutical composition of paragraph 8, wherein the inhibitor of AFP and MHC Class I-related molecule interactions inhibits interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

20. The pharmaceutical composition of paragraph 8, wherein the inhibitor of AFP and MHC Class I-related molecule interactions inhibits interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

21. The pharmaceutical composition of paragraph 8, wherein the inhibitor of AFP-MHC Class I-related interactions inhibits interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

22. The pharmaceutical composition of paragraph 8, wherein the inhibitor of AFP-MHC Class I-related interactions inhibits interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

23. The pharmaceutical composition of paragraph 8, wherein the inhibitor of AFP-MHC Class I-related interactions inhibits interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

24. The pharmaceutical composition of paragraph 8, wherein the inhibitor of AFP-MHC Class I-related interactions inhibits interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

25. The pharmaceutical composition of paragraph 8, wherein the inhibitor of AFP-MHC Class I-related interactions inhibits interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

26. The pharmaceutical composition of paragraph 8, wherein the inhibitor of AFP-MHC Class I-related interactions inhibits interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

27. The pharmaceutical composition of paragraph 8, wherein the inhibitor of AFP-MHC Class I-related interactions inhibits interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

28. The pharmaceutical composition of paragraph 8, wherein the inhibitor of AFP-MHC Class I-related interactions inhibits interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

29. The pharmaceutical composition of paragraph 8, wherein the inhibitor of AFP-MHC Class I-related interactions inhibits interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

30. The pharmaceutical composition of paragraph 8, wherein the inhibitor of AFP-MHC Class I-related interactions inhibits interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

31. The pharmaceutical composition of paragraph 8, wherein the inhibitor of AFP-MHC Class I-related interactions inhibits interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

32. The pharmaceutical composition of paragraph 8, wherein the inhibitor of AFP-MHC Class I-related interactions inhibits interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

33. The pharmaceutical composition of any one of paragraphs 8-32, wherein the inhibitor of AFP-MHC Class I-related interactions also inhibits binding between S527 or D528 of SEQ ID NO: 2 and E50 and 67Y of β2M, respectively, complexed with an MHC Class I-related molecule.

34. The pharmaceutical composition of any one of paragraphs 8-33, wherein the inhibitor of AFP-MHC Class I-related interactions also inhibits binding between R604 of SEQ ID NO: 2 and the carbonyl oxygen at E50 of β2M, wherein the β2M is complexed with an MHC Class I-related molecule.

35. The pharmaceutical composition of any one of paragraphs 8-34, wherein the inhibitor of AFP-MHC Class I-related interactions is an antibody or antigen-binding fragment thereof, a small molecule compound, a peptide inhibitor, or an RNA or DNA aptamer.

36. The pharmaceutical composition of paragraph 35, wherein the antibody or antigen-binding fragment thereof is a chimeric, humanized, or completely human antibody or antigen-binding fragment thereof.

37. A pharmaceutical composition comprising a potentiator of alpha-fetoprotein (AFP)-β2M (β-2-microglobulin interactions and a pharmaceutically acceptable carrier, wherein said potentiator of AFP-β2M interactions increases binding between AFP and β2M.

38. The pharmaceutical composition of paragraph 37, wherein the potentiator of AFP-β2M interactions increases interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO:4, an interface of β2M comprising amino acids 81-96 of SEQ ID NO: 4, or any combination thereof.

39. The pharmaceutical composition of paragraph 37, wherein the potentiator of AFP-β2M interactions increases interaction of AFP with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

40. The pharmaceutical composition of paragraph 37, wherein the increased binding between AFP and β2M further increases or enhances interaction or complex formation between β2M and an MHC Class I-related molecule.

41. The pharmaceutical composition of paragraph 40, wherein the MHC Class I-related molecule is selected from HFE, HLA-A, HLA-G, HLA-E, HLA-B, MR1, CD1D, HLA-C, ZA2G, CD1A, and CD1B.

42. The pharmaceutical composition of any one of paragraphs 32-36, wherein the potentiator of AFP-β2M interactions is an antibody or antigen-binding fragment thereof, a small molecule compound, or an RNA or DNA aptamer.

43. The pharmaceutical composition of paragraph 42, wherein the antibody or antigen-binding fragment thereof is a chimeric, humanized, or completely human antibody or antigen-binding fragment thereof.

44. A pharmaceutical composition comprising a potentiator of AFP-MHC Class I-related molecule interactions and a pharmaceutically acceptable carrier, wherein said potentiator of AFP-MHC Class I-related molecule interactions increases binding between alpha-fetoprotein (AFP) and an MHC Class I-related molecule.

45. The pharmaceutical composition of paragraph 44, wherein the MHC Class I-related molecule is selected from HFE, HLA-A, HLA-G, HLA-E, HLA-B, MR1, CD1D, HLA-C, ZA2G, CD1A, and CD1B.

46. The pharmaceutical composition of paragraph 44, wherein the potentiator of AFP-MHC Class I-related molecule interactions increases interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

47. The pharmaceutical composition of paragraph 44, wherein the potentiator of AFP-MHC Class I-related molecule interactions interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

48. The pharmaceutical composition of paragraph 39, wherein the potentiator of AFP-MHC Class I-related interactions increases interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

49. The pharmaceutical composition of paragraph 44, wherein the potentiator of AFP-MHC Class I-related molecule interactions increases interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

50. The pharmaceutical composition of paragraph 44, wherein the potentiator of AFP-MHC Class I-related molecule interactions increases interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

51. The pharmaceutical composition of paragraph 44, wherein the potentiator of AFP-MHC Class I-related molecule interactions interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

52. The pharmaceutical composition of paragraph 44, wherein the potentiator of AFP-MHC Class I-related molecule interactions increases interaction of AFP with an interface of MR1 comprising amino acids 40-67 of SEQ ID NO: 22, amino acids 148-180 of SEQ ID NO: 22, or amino acids 40-67 and 148-180 of SEQ ID NO: 22.

53. The pharmaceutical composition of paragraph 44, wherein the potentiator of AFP-MHC Class I-related molecule interactions increases interaction of AFP with an interface of ZA2G comprising amino acids 45-72 of SEQ ID NO: 18, amino acids 152-183 of SEQ ID NO: 18, or amino acids 45-72 and 152-183 of SEQ ID NO: 18.

54. The pharmaceutical composition of paragraph 44, wherein the potentiator of AFP-MHC Class I-related molecule interactions increases interaction of AFP with an interface of CD1A comprising amino acids 41-71 of SEQ ID NO: 24, amino acids 153-183 of SEQ ID NO: 24, or amino acids 41-71 and 153-183 of SEQ ID NO: 24.

55. The pharmaceutical composition of paragraph 44, wherein the potentiator of AFP-MHC Class I-related molecule interactions increases interaction of AFP with an interface of CD1B comprising amino acids 41-71 of SEQ ID NO: 26, amino acids 156-185 of SEQ ID NO: 26, or amino acids 41-71 and 156-185 of SEQ ID NO: 26.

56. The pharmaceutical composition of paragraph 44, wherein the potentiator of AFP-MHC Class I-related molecule interactions increases interaction of AFP with an interface of CD1D comprising amino acids 45-71 of SEQ ID NO: 30, amino acids 153-184 of SEQ ID NO: 30, or amino acids 45-71 and 153-184 of SEQ ID NO: 30.

57. The pharmaceutical composition of paragraph 44, wherein the potentiator of AFP-MHC Class I-related molecule interactions increases interaction of HLA-A with an interface of AFP comprising amino acids 131-136 of SEQ ID NO: 2, amino acids 440-449 of SEQ ID NO: 2, amino acids 484-493 of SEQ ID NO: 2, amino acids 520-558 of SEQ ID NO: 2, or any combination thereof.

58. The pharmaceutical composition of paragraph 44, wherein the potentiator of AFP-MHC Class I-related molecule interactions increases interaction of HLA-B with an interface of AFP comprising amino acids 133-135 of SEQ ID NO: 2, amino acids 440-446 of SEQ ID NO: 2, amino acids 484-493 of SEQ ID NO: 2, amino acids 520-558 of SEQ ID NO: 2, or any combination thereof.

59. The pharmaceutical composition of paragraph 44, wherein the potentiator of AFP-MHC Class I-related molecule interactions increases interaction of HLA-C with an interface of AFP comprising amino acids 105-112 and 135 of SEQ ID NO: 2, amino acids 440-446 of SEQ ID NO: 2, amino acids 483-493 of SEQ ID NO: 2, amino acids 520-558 of SEQ ID NO: 2, or any combination thereof.

60. The pharmaceutical composition of paragraph 44, wherein the potentiator of AFP-MHC Class I-related molecule interactions increases interaction of HLA-E with an interface of AFP comprising amino acids 105-112 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

61. The pharmaceutical composition of paragraph 44, wherein the potentiator of AFP-MHC Class I-related molecule interactions increases interaction of HLA-G with an interface of AFP comprising amino acids 105-112 and 131-135 of SEQ ID NO: 2, amino acids 440-449 of SEQ ID NO: 2, amino acids 483-493 of SEQ ID NO: 2, amino acids 520-558 of SEQ ID NO: 2, or any combination thereof.

62. The pharmaceutical composition of paragraph 44, wherein the potentiator of AFP-MHC Class I-related molecule interactions increases interaction of HFE with an interface of AFP comprising amino acids 105-112 and 133-135 of SEQ ID NO: 2, amino acids 440-449 of SEQ ID NO: 2, amino acids 487-495 of SEQ ID NO: 2, amino acids 520-558 of SEQ ID NO: 2, or any combination thereof.

63. The pharmaceutical composition of paragraph 44, wherein the potentiator of AFP-MHC Class I-related molecule interactions increases interaction of MR1 with an interface of AFP comprising amino acids 105-107 and 131-135 of SEQ ID NO: 2, amino acids 441-449 of SEQ ID NO: 2, amino acids 484-495 of SEQ ID NO: 2, amino acids 520-552 of SEQ ID NO: 2, or any combination thereof.

64. The pharmaceutical composition of paragraph 44, wherein the potentiator of AFP-MHC Class I-related molecule interactions increases interaction of ZA2G with an interface of AFP comprising amino acids 105-115 and 131-137 of SEQ ID NO: 2, amino acids 440-446 of SEQ ID NO: 2, amino acids 487-493 of SEQ ID NO: 2, amino acids 520-558 of SEQ ID NO: 2, or any combination thereof.

65. The pharmaceutical composition of paragraph 44, wherein the potentiator of AFP-MHC Class I-related molecule interactions increases interaction of CD1A with an interface of AFP comprising amino acids 105-112 and 130-137 of SEQ ID NO: 2, amino acids 441-449 of SEQ ID NO: 2, amino acids 483-493 of SEQ ID NO: 2, amino acids 521-552 of SEQ ID NO: 2, or any combination thereof.

66. The pharmaceutical composition of paragraph 44, wherein the potentiator of AFP-MHC Class I-related molecule interactions increases interaction of CD1B with an interface of AFP comprising amino acids 105-112 and 130-137 of SEQ ID NO: 2, amino acids 440-449 of SEQ ID NO: 2, amino acids 484-493 of SEQ ID NO: 2, amino acids 520-552 of SEQ ID NO: 2, or any combination thereof.

67. The pharmaceutical composition of paragraph 44, wherein the potentiator of AFP-MHC Class I-related molecule interactions increases interaction of CD1D with an interface of AFP comprising amino acids 105-112 and 131-137 of SEQ ID NO: 2, amino acids 441-449 of SEQ ID NO: 2, amino acids 483-493 of SEQ ID NO: 2, amino acids 520-539 of SEQ ID NO: 2, or any combination thereof.

68. The pharmaceutical composition of any one of paragraphs 44-67, wherein the potentiator of AFP-MHC Class I-related interactions also increases interaction between S527 or D528 of SEQ ID NO: 2 and E50 and 67Y of SEQ ID NO: 4, respectively, complexed with an MHC Class I-related molecule.

69. The pharmaceutical composition of any one of paragraphs 44-68, wherein the potentiator of AFP-MHC Class I-related interactions also increases interaction between R604 of SEQ ID NO: 2 and the carbonyl oxygen at E50 of SEQ ID NO: 4, wherein the β2M is complexed with an MHC Class I-related molecule.

70. The pharmaceutical composition of any one of paragraphs 44-69, wherein the potentiator of AFP-MHC Class I-related interactions is an antibody or antigen-binding fragment thereof, a small molecule compound, or an RNA or DNA aptamer.

71. The pharmaceutical composition of paragraph 70, wherein the antibody or antigen-binding fragment thereof is a chimeric, humanized, or completely human antibody or antigen-binding fragment thereof.

72. A method to inhibit or reduce alpha-fetoprotein (AFP) and β2M (β-2-microglobulin) interactions in a disease or disorder associated with AFP-mediated immunosuppression comprising administering a therapeutically effective amount of a pharmaceutical composition comprising an inhibitor of AFP-β2M interactions and a pharmaceutically acceptable carrier of any one of paragraphs 1-7 to a subject in need thereof.

73. A method to inhibit or reduce alpha-fetoprotein (AFP) and MHC Class I-related interactions in a disease or disorder associated with AFP-mediated immunosuppression comprising administering a therapeutically effective amount of a pharmaceutical composition comprising an inhibitor of AFP-MHC Class I-related interactions of any one of paragraphs 8-36 to a subject in need thereof.

74. The method of any one of paragraphs 72-73, wherein the subject has or has been diagnosed with cancer.

75. The method of any one of paragraphs 72-74, further comprising administering an anti-cancer therapy or agent to the subject.

76. The method of any one of paragraphs 72-75, further comprising administering a tumor or cancer antigen.

77. The method of any one of paragraphs 72-73, wherein the subject has or has been diagnosed with a chronic immune infection.

78. A method to increase or potentiate alpha-fetoprotein (AFP) and β2M (β-2-microglobulin) interactions in diseases or disorders associated with decreased AFP levels or where increasing AFP levels is beneficial comprising administering a therapeutically effective amount of a pharmaceutical composition comprising a potentiator of AFP-β2M interactions of any one of paragraphs 37-43 to a subject in need thereof.

79. A method to increase or potentiate alpha-fetoprotein (AFP) and MHC Class I-related interactions in diseases or disorders associated with decreased AFP levels or where increasing AFP levels is beneficial comprising administering a therapeutically effective amount of a pharmaceutical composition comprising a potentiator of AFP-MHC Class I-related interactions of any one of paragraphs 44-71 to a subject in need thereof.

80. The method of any one of paragraphs 78-79, wherein the subject has or has been diagnosed with an autoimmune disease or disorder.

81. The method of any one of paragraphs 78-79, wherein the subject has or has been diagnosed with host versus graft disease (HVGD), is an organ or tissue transplant recipient, or a recipient of an allogenic transplant.

82. The method any one of paragraphs 78-79, wherein the subject has had an organ transplantation, partial resection of an organ or other organ injury and is in need of enhanced organ regeneration.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

As described in International Application No. PCT/US15/26860, the contents of which are herein incorporated by reference in their entireties, we previously discovered and disclosed that alphafetoprotein was a third ligand for the neonatal Fc receptor (FcRn). In modeling this interaction, we recognized that AFP possessed the novel property among other FcRn ligands (albumin and IgG) of exhibiting extensive potential interactions with beta 2-microglobulin (β2M). This included AFP residues S527 and D528 (in single letter amino acid code) contacts with E50 and Y67 of β2M. Similarly, AFP residue R604 is predicted to interact with the carbonyl oxygen of E50 of β2M Further, these contact sites are predicted to be increased at acidic pH. Overall, the docking models indicated that AFP makes strong interactions with β2M. PDB-PISA analysis of the AFP:β2M docking model predicts a high interface area of interactions that range from 1000-1500 angstroms and a 8-11 kcal/mol gain in solvation free energy upon binding consistent with a favorable interaction.

As β2M is a nonpolymorphic molecule that noncovalently associates with many MHC class I related molecules in addition to FcRn, we investigated whether AFP interacted with other MHC class I related molecules as well. We considered therefore that there were two classes of binding sites between AFP and potential partners: the β2M-associated binding site on AFP which was fixed, and a group of sites involved in interactions with MHC class I related heavy chains that non-covalently associate with β2M. We therefore modeled FcRn relatedness to other MHC class I related heavy chains. We searched for percent amino acid identity between different members of the MHC class I family and established a phylogenetic tree (cladogram) of different members of the MHC class I family. This established a potential hierarchy of relatedness to FcRn: HFE≥HLA-A≥HLA-G≥HLA-E≥HLA-B≥MR1≥CD1D≥HLA-C≥ZA2G≥CD1A≥CD1B. Because MR1 is the restriction element for mucosa associated invariant T cells (MAIT), we further modeled the interactions between AFP and MR1 based upon published crystal structures. We found that the MR1 crystal structure (PDB ID 5D5M) can be superimposed on the FcRn crystal structure (PDB 4N0U) with an RMSD of 2.8 indicating a high degree of similarity. Further the modeling predicted a strong interaction between AFP and MR1 with a 1278 angstrom interface area with a 6.4 kcal/mol gain in free energy upon binding. These studies therefore predict in their totality that AFP interacts broadly with MHC class I related molecules through a common β2M anchoring interaction and a variable level of MHC class I related heavy chain interactions that depend upon the specific MHC class I related molecule involved. These observations are consistent with the broad immunosuppressive but unexplained properties of AFP and point toward unique strategies for blocking the immunosuppressive function of AFP. Specifically, these analyses indicate that AFP can be targeted by various combinations of focusing on inhibiting the β2M and heavy chain docking sites which are each about 1000-1500 angstroms in size and vary between MHC class I related molecules; all possess a similar architecture of docking, but through different amino acid interactions.

First, we have found that human AFP can bind directly to human β2M on a sensor chip in BIACORE™ surface plasmon resonance experiments with an overall K_{D} of approximately 12.5. This is compared to our observed K_{D} between AFP and FcRn, for example, which has an at least 100 fold higher affinity, consistent with the hypothesis that there is synergy between the heavy chain and β2M docking sites on AFP. In a similar manner, β2M in the eluate binds to AFP on the sensor. Therefore, β2M binds to the AFP in either orientation in SPR experiments. To further confirm this, we have found that preincubation of human peripheral blood mononuclear cells or spleen cells of humanized mice (mice deficient in mouse FcRn but transgenic for human FcRn and human β2M) with AFP decreases detection of human β2M by anti-β2M antibodies (in comparison to human serum albumin which does not). This indicates that AFP binding to β2M obscures β2M interactions with antibodies to this subunit (consistent with an interaction) or binds to β2M and forces its internalization. Either way, this is further consistent with an interaction between AFP and native β2M on primary cells.

1.-82. (canceled)

83. A pharmaceutical composition comprising an inhibitor of alpha-fetoprotein (AFP)-β2-microglobulin (β2M) interactions and a pharmaceutically acceptable carrier, wherein said inhibitor of AFP-β2M interactions inhibits binding between AFP and β2M.

84. The pharmaceutical composition of claim 83, wherein the inhibitor of AFP-β2M interactions inhibits interaction of AFP with: an interface of β2M comprising amino acids 1-9 of SEQ ID NO: 4, an interface of β2M comprising amino acids 24-36 of SEQ ID NO: 4, an interface of β2M comprising amino acids 42-65 of SEQ ID NO: 4, an interface of β2M comprising amino acids 81-96 of SEQ ID NO: 4, or any combination thereof.

85. The pharmaceutical composition of claim 83, wherein the inhibitor of AFP-β2M interactions inhibits interaction of β2M with: an interface of AFP comprising amino acids 105-112 and 131-138 of SEQ ID NO: 2, an interface of AFP comprising amino acids 440-453 of SEQ ID NO: 2, an interface of AFP comprising amino acids 483-493 of SEQ ID NO: 2, an interface of AFP comprising amino acids 519-560 of SEQ ID NO: 2, or any combination thereof.

86. The pharmaceutical composition of claim 83, wherein the inhibition of binding between AFP and β2M further inhibits or prevents interaction or complex formation between β2M and an MHC Class I-related molecule.

87. The pharmaceutical composition of claim 86, wherein the MHC Class I-related molecule is selected from HFE, HLA-A, HLA-G, HLA-E, HLA-B, MR1, CD1D, HLA-C, ZA2G, CD1A, CD1B.

88. The pharmaceutical composition of any one of claim 83, wherein the inhibitor of AFP-β2M interactions is an antibody or antigen-binding fragment thereof, a small molecule compound, or an RNA or DNA aptamer.

89. The pharmaceutical composition of claim 88, wherein the antibody or antigen-binding fragment thereof is a chimeric, humanized, or completely human antibody or antigen-binding fragment thereof.

90. A pharmaceutical composition comprising an inhibitor of alpha-fetoprotein (AFP)-MHC Class I-related interactions and a pharmaceutically acceptable carrier, wherein said inhibitor of AFP-MHC Class I-related interactions inhibits binding between AFP and an MHC Class I-related molecule.

100. The pharmaceutical composition of claim 90, wherein the MHC Class I-related molecule is selected from HFE, HLA-A, HLA-G, HLA-E, HLA-B, MR1, CD1D, HLA-C, ZA2G, CD1A, and CD1B.

101. The pharmaceutical composition of claim 90, wherein the inhibitor of AFP-MHC Class I-related interactions inhibits interaction of AFP with an interface of HLA-A comprising amino acids 41-68 of SEQ ID NO: 6, amino acids 154-181 of SEQ ID NO: 6, or amino acids 41-68 and 154-181 of SEQ ID NO: 6.

102. The pharmaceutical composition of claim 90, wherein the inhibitor of AFP-MHC Class I-related interactions inhibits interaction of AFP with an interface of HLA-B comprising amino acids 41-68 of SEQ ID NO: 8, amino acids 143-183 of SEQ ID NO: 8, or amino acids 41-68 and 143-183 of SEQ ID NO: 8.

103. The pharmaceutical composition of claim 90, wherein the inhibitor of AFP-MHC Class I-related interactions inhibits interaction of AFP with an interface of HLA-C comprising amino acids 41-68 of SEQ ID NO: 10, amino acids 154-182 of SEQ ID NO: 10, or amino acids 41-68 and 154-182 of SEQ ID NO: 10.

104. The pharmaceutical composition of claim 90, wherein the inhibitor of AFP-MHC Class I-related interactions inhibits interaction of AFP with an interface of HLA-E comprising amino acids 41-68 of SEQ ID NO: 12, amino acids 154-181 of SEQ ID NO: 12, or amino acids 41-68 and 154-181 of SEQ ID NO: 12.

105. The pharmaceutical composition of claim 90, wherein the inhibitor of AFP-MHC Class I-related interactions inhibits interaction of AFP with an interface of HLA-G comprising amino acids 41-68 of SEQ ID NO: 16, amino acids 154-181 of SEQ ID NO: 16, or amino acids 41-68 and 154-181 of SEQ ID NO: 16.

106. The pharmaceutical composition of claim 90, wherein the inhibitor of AFP-MHC Class I-related interactions inhibits interaction of AFP with an interface of HFE comprising amino acids 42-70 of SEQ ID NO: 20, amino acids 152-179 of SEQ ID NO: 20, or amino acids 42-70 and 152-179 of SEQ ID NO: 20.

107. A method to inhibit or reduce alpha-fetoprotein (AFP) and β2M (β-2-microglobulin) interactions in a disease or disorder associated with AFP-mediated immunosuppression comprising administering a therapeutically effective amount of a pharmaceutical composition comprising an inhibitor of AFP-β2M interactions and a pharmaceutically acceptable carrier of any one of claim 83 to a subject in need thereof.

108. The method of any one of claim 98, wherein the subject has or has been diagnosed with cancer.

109. The method of any one of claim 98, further comprising administering an anti-cancer therapy or agent to the subject.

110. The method of any one of claim 98, further comprising administering a tumor or cancer antigen.

111. The method of any one of claim 98, wherein the subject has or has been diagnosed with a chronic infection.
